(19) United States
(12) Patent Application Publication
(10) Pub. No.: US 2026/0167737 A1
(43) Pub. Date: Jun. 18, 2026

(54) ANTI-CDH17 ANTIBODIES AND USE OF THE SAME

(71) Applicant: BigHat Biosciences, Inc., San Mateo, CA (US)

(72) Inventors: Mark DEPRISTO, Los Altos, CA (US); Peyton GREENSIDE, San Mateo, CA (US); Ryan HENRICI, Philadelphia, PA (US)

(21) Appl. No.: 19/151,550

(22) PCT Filed: Jan. 31, 2024
(86) PCT No.: PCT/US2024/013798

§ 371 Date: Jul. 28, 2025

Related U.S. Application Data
(60) Provisional application No. 63/442,389, filed on Jan. 31, 2023.

(51) Int. Cl. C07K 16/28; A61K 47/68; A61P 35/00

(52) U.S. Cl. CPC C07K 16/2896; A61K 47/68031; A61K 47/6849; A61K 47/6889; A61P 35/00; C07K 2317/31; C07K 2317/52; C07K 2317/569; C07K 2317/73

(57) ABSTRACT

Described herein are anti-CDH17 antibodies and use of the same. The anti-CDH17 antibodies provided herein exhibit significantly improved binding affinity after optimization.

### CROSS REFERENCE

This application claims the benefit of U.S. Provisional Application No. 63/442,389, filed on Jan. 31, 2023, which is incorporated herein by reference in its entirety.

### BACKGROUND OF THE INVENTION

Antibody driven targeted cancer immunotherapy has played a major role in cancer immunotherapy. By targeting surface antigen expressed on tumor cells, the antibodies have demonstrated efficacy in the targeting and killing of tumor cells.

### SUMMARY OF THE INVENTION

Provided herein is an anti-CDH17 antibody or antigen-binding fragment thereof comprising (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75.

Provided herein is an anti-CDH17 antibody or antigen-binding fragment thereof comprising (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75.

Provided herein is an anti-CDH17 antibody or antigen-binding fragment thereof comprising a HCDR1 as set out in SEQ ID NO: 41, a HCDR2 as set out in SEQ ID NO: 43, and/or a HCDR3 as set out in SEQ ID NO: 62.

Provided herein is an anti-CDH17 antibody or antigen-binding fragment thereof comprising a HCDR1 as set out in SEQ ID NO: 41, a HCDR2 as set out in SEQ ID NO: 44, and/or a HCDR3 as set out in SEQ ID NO: 63.

Provided herein is an anti-CDH17 antibody or antigen-binding fragment thereof comprising a HCDR1 as set out in SEQ ID NO: 41, a HCDR2 as set out in SEQ ID NO: 43, and/or a HCDR3 as set out in SEQ ID NO: 64.

Provided herein is an anti-CDH17 antibody or antigen-binding fragment thereof comprising a HCDR1 as set out in SEQ ID NO: 41, a HCDR2 as set out in SEQ ID NO: 45, and/or a HCDR3 as set out in SEQ ID NO: 65.

Provided herein is an anti-CDH17 antibody or antigen-binding fragment thereof comprising a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6.

Provided herein is an anti-CDH17 antibody or antigen-binding fragment thereof comprising a HCDR1 as set out in SEQ ID NO: 41, a HCDR2 as set out in SEQ ID NO: 47, and/or a HCDR3 as set out in SEQ ID NO: 72.

Provided herein is an anti-CDH17 antibody or antigen-binding fragment thereof comprising a HCDR1 as set out in SEQ ID NO: 41, a HCDR2 as set out in SEQ ID NO: 46, and/or a HCDR3 as set out in SEQ ID NO: 65.

Provided herein is an anti-CDH17 antibody or antigen-binding fragment thereof comprising at least one amino acid substitutions at position 25-106 in an amino acid sequence as set out in SEQ ID NO: 1.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L.

Provided herein is an anti-CDH17 antibody or antigen-binding fragment thereof, comprising (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises a T67V mutation in an amino acid sequence as set out in SEQ ID NO: 1.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, Xu is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AKSVRX₂₂ (SEQ ID NO: 99), wherein X₁₉ is H or Y, X₂₀ is G, T, V, K or N, and X₂₂ is S, L, K or A.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A.

Provided herein is an anti-CDH17 antibody or antigen-binding fragment thereof, wherein the anti-CDH17 antibody or antigen-binding fragment thereof has an increased binding affinity to CDH17 compared to a binding affinity of an amino acid sequence as set out in SEQ ID NO: 1 to CDH17.

Provided herein is an anti-CDH17 antibody or antigen-binding fragment thereof, wherein the anti-CDH17 antibody or antigen-binding fragment thereof has increased stability compared to stability of an amino acid sequence as set out in SEQ ID NO: 1.

Provided herein is an anti-CDH17 antibody or antigen-binding fragment thereof, wherein the anti-CDH17 antibody or antigen-binding fragment thereof has a higher purity compared to a purity of an amino acid sequence as set out in SEQ ID NO: 1.

Provided herein is a multi-specific binding molecule comprising a first binding domain and a second binding domain, wherein the first binding domain comprises the anti-CDH17 antibody or antigen-binding fragment thereof.

Provided herein is a protein comprising the anti-CDH17 antibody or antigen-binding fragment thereof conjugated to a cytotoxic agent, and/or a Fc domain.

Provided herein is a conjugate comprising Formula (I): Ab-(L-D)n wherein Ab comprises an anti-CDH17 antibody or antigen-binding fragment thereof, wherein the anti-CDH17 antibody or antigen-binding fragment thereof comprises (a) a HCDR1 having at least 80% sequence identity to the amino acid sequence set out in SEQ ID NO: 41, (b) a HCDR2 having at least 80% sequence identity to the amino acid sequence set out in any one of SEQ ID NOs: 43-60, and (c) a HCDR3 having at least 80% sequence identity to the amino acid sequence set out in any one of SEQ ID NOs: 61-75; L is a linker; D is a cytotoxic agent; and n is a drug antibody ratio, wherein the drug antibody ratio is from about 1 to about 8.

Provided herein is a nucleic acid encoding an anti-CDH17 antibody or antigen-binding fragment thereof, a multi-specific binding molecule described herein, a protein disclosed herein, or a conjugate described herein.

Provided herein is an expression vector comprising a nucleic acid encoding an anti-CDH17 antibody or antigen-binding fragment thereof disclosed herein, a multi-specific binding molecule disclosed herein, or a protein disclosed herein.

Provided herein is a host cell comprising a nucleic acid or an expression vector encoding an anti-CDH17 antibody or antigen-binding fragment thereof disclosed herein, a multi-specific binding molecule disclosed herein, or a protein disclosed herein.

Provided herein is a method of producing an anti-CDH17 antibody or antigen-binding fragment thereof disclosed herein, a multi-specific binding molecule disclosed herein, or a protein disclosed herein, the method comprising maintaining the host cell in a medium to produce the anti-CDH17 antibody or antigen-binding fragment thereof, the multi-specific binding molecule, the protein and isolating or purifying the anti-CDH17 antibody or antigen-binding fragment thereof, the multi-specific binding molecule, the protein produced by the host cell.

Provided herein is a pharmaceutical composition for treating cancer, comprising an anti-CDH17 antibody or antigen-binding fragment thereof disclosed herein, a-specific binding molecule disclosed herein, a protein disclosed herein, a nucleic acid disclosed herein, an expression vector or a host cell disclosed herein and a pharmaceutically acceptable carrier or excipient.

Provided herein is a method for treating cancer in a subject in need thereof comprising administering to the subject a therapeutically effective amount of an anti-CDH17 antibody or antigen-binding fragment thereof disclosed herein, a multi-specific binding molecule disclosed herein, a protein disclosed herein, a nucleic acid disclosed herein, an expression vector disclosed herein, a host cell disclosed herein, or a pharmaceutical composition disclosed herein.

Provided herein is use of an anti-CDH17 antibody or antigen-binding fragment thereof disclosed herein, a multi-specific binding molecule disclosed herein, a protein disclosed herein, ta nucleic acid disclosed herein, an expression vector disclosed herein, a host cell disclosed herein, or a pharmaceutical composition disclosed herein in the manufacture of a medicament for treatment of cancer.

Provided herein is a kit comprising an anti-CDH17 antibody or antigen-binding fragment thereof disclosed herein, a multi-specific binding molecule disclosed herein, a protein disclosed herein, a nucleic acid disclosed herein, an expression vector disclosed herein, a host cell disclosed herein, or a pharmaceutical composition disclosed herein, and instructions for use.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C shows exemplary multi specific T cell engager designs demonstrating the possible arrangement of an VHH and an scFv or Fab.

FIG. 2 shows an exemplary Bispecific T cell engager construct lacking an Fc domain (1+1 geometry) can be produced in CHO cells with high purity by SEC-HPLC.

FIG. 3 shows asymmetric an exemplary Bispecific T cell engager construct with an Fc domain (2+1 geometry) can be produced in CHO cells with high purity by SEC-HPLC.

FIG. 4 shows symmetric an exemplary Bispecific T cell engager construct with an Fc domain (2+2 geometry) can be produced in CHO cells with high purity by SEC-HPLC.

FIG. 5 shows an exemplary Bispecific T cell engager construct readily induce T cell dependent cellular cytotoxicity (TDCC) in co-cultures of primary human T cells and AsPC-1 pancreatic adenocarcinoma cells (10:1 T cell:target cell ratio).

FIG. 6 shows VHH-Fc fusion protein can be produced in CHO cells with high purity by SEC-HPLC.

FIG. 7 shows VHH-Fc fusion proteins can be readily conjugated to cytotoxic linker payloads (e.g., vc-PAB-MMAE) via reduction of interchain disulfide bonds and maleimide chemistry with an average DAR of 2.0 and high post-conjugation purity by SEC-HPLC.

FIG. 8 shows CDH17 VHH-Fc fusion proteins conjugated to mc-vc-PAB-MMAE induce potent cytotoxicity against representative CDH17+ cancer cell lines in vitro with low pM IC₅₀. Unconjugated CDH17-directed VHH-Fc fusions are not cytotoxic.

FIG. 9 shows a comparison of cytotoxicity of CDH17-VHH-0156-Fc (SEQ ID NO: 108) and parental VHH-0001-Fc fusion protein (SEQ ID NO: 107) mc-vc-PAB-MMAE conjugates (DAR2) in SNU16 cells.

FIG. 10 shows CDH17 expression in primary tumor biopsy core samples assessed by immunohistochemistry (IHC).

FIG. 11A-11B shows representative in vitro cancer cell drug sensitivity plots for BHB 156-vedotin (DAR4) against representative CDH17+ cancer cell lines (FIG. 11A) and primary patient derived xenograft (PDX) cancer cell tissues (FIG. 11B).

FIG. 12 shows a summary table of cell line sensitivities by IC₅₀ estimates derived from serial dilutions of BHB 156-vedotin (DAR4) or isotype control vedotin ADCs in vitro alongside receptor density measurements by antibody binding capacity (ABC) of CDH17 molecules per cell surface.

FIG. 13 shows a summary table of cell line sensitivities by IC₅₀ estimates derived from serial dilutions of BHB 156-vedotin (DAR4) or isotype control vedotin ADCs in vitro alongside receptor density measurements by antibody binding capacity (ABC) of CDH17 molecules per cell surface and IHC score.

FIG. 14 shows a representative plot and summary table of plasma elimination of a single dose of I-125 radiolabeled BHB156 or anti-SARS-CoV-2 isotype control VHH-Fc fusion protein in Sprague-Dawley rats at various concentrations.

FIG. 15 shows a representative biodistribution of BHB156 or isotype control VHH-Fc from a single dose of 20 mg/kg I-125 radiolabeled BHB156 or anti-SARS-CoV-2 isotype control VHH-Fc fusion protein in Sprague-Dawley rats assessed 3 or 10 days after administration.

FIG. 16A-16D shows the potency of BHB 156-vedotin (DAR4) versus isotype vedotin in clearing tumor biomass in cell line derived xenograft (CDX) tumor-bearing mice.

### DETAILED DESCRIPTION OF THE INVENTION

### Overview

Described herein, in some aspects, is an anti-CDH17 antibody or antigen-binding fragment thereof, comprising at least one genetic modification, wherein the at least one genetic modification results in substantially improved binding affinity and increased inhibition in CDH17 activity. In some aspects, the anti-CDH17 antibody may comprise at least one genetic modification resulting in substitution, deletion or insertion of at least one amino acid of a polypeptide that encodes anti-CDH17 or antigen-binding fragment thereof.

In some aspects, the anti-CDH17 antibodies or antigen-binding fragments thereof provided herein may be Fab, Fab′, Fab′-SH, F(ab′)2, Fv, TaFv, scFv, diabody, bsDb, scDb, DART, BiTE, and VHH fragments. For example, the anti-CDH17 antibodies or antigen-binding fragment thereof described herein may be a multi-specific antigen-binding protein (e.g., bispecific T-cell engaging antibody) which binds to the antigen of interest, e.g., a protein, with sufficient affinity such that the multi-specific antigen-binding protein may be used as a diagnostic and/or therapeutic agent in targeting a protein or a cell, tissue or tumor expressing the protein, and does not significantly cross-react with other proteins. The present disclosure may be bispecific antibodies (BsAbs) capable of binding to CD3 and CDH17.

In some aspects, the anti-CDH17 antibody provided herein may comprise an anti-CDH17 antibody or fragment thereof and a moiety (e.g., a cytotoxic agent, a Fc domain). For example, the anti-CDH17 antibody or fragment thereof composition may be an anti-CDH17 antibody or antigen-binding fragment thereof conjugated via a linker (L) to a cytotoxic agent and/or a Fc domain. Also described herein are methods for treating cancer in a subject in need thereof comprising administering to the subject a therapeutically effective amount of the anti-CDH17 antibody or antigen-binding fragment thereof compositions.

Anti-CDH17 immunotherapies can have a significant promise as treatments for a multitude of hematological and solid tissue malignancies. Recognized herein is an unmet need to engineer anti-CDH17 antibody compositions with enhanced binding affinity to CDH17. Disclosed herein are methods to engineer anti-CDH17 antibody compositions with enhanced binding affinity to CDH17.

Provided herein is an anti-CDH17 antibody or antigen-binding fragment thereof comprising (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, ssRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, use of the anti-CDH17 antibody or antigen-binding fragment thereof, the multi-specific binding molecule, the protein, the conjugate, the nucleic acid, the expression vector, the host cell, or the pharmaceutical composition in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for use in diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, ssRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising a HCDR1 as set out in SEQ ID NO: 41, a HCDR2 as set out in SEQ ID NO: 45, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising a HCDR1 as set out in SEQ ID NO: 41, a HCDR2 as set out in SEQ ID NO: 47, and/or a HCDR3 as set out in SEQ ID NO: 72. In some embodiments, the anti-CDH17 antibody or antigen-binding fragment thereof comprises an amino acid sequence identity to the amino acid sequence set out in SEQ ID NO: 109. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, I N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition is for diagnosis of cancer. In some embodiments, the cancer is gastric, pancreatic, neuroendocrine, or colorectal cancer.

Provided herein is an anti-CDH17 antibody or antigen-binding fragment thereof, comprising (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X, is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, Xu is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, ssRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, ssRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, ssRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof, wherein the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10-9M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof, wherein the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ a HCDR2 ID NO: 41); (b) having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) HCDR2 an a having amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) having a HCDR2 an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is an anti-CDH17 antibody or antigen-binding fragment thereof, wherein the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X, is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) HCDR2 an a having amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, Xu is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xin is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, ssRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is a multi-specific binding molecule comprising a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX, (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, Xu is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having amino acid an sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof. In some embodiments, the anti-CDH17 antibody or antigen-binding fragment thereof is a scFv antibody. In some embodiments, the anti-CDH17 antibody or antigen-binding fragment thereof is a VHH antibody. In some embodiments, the anti-CDH17 antibody or antigen-binding fragment thereof is humanized. In some embodiments, the anti-CDH17 antibody or antigen-binding fragment thereof is nonhuman antibody. In some embodiments, the anti-CDH17 antibody or antigen-binding fragment thereof is a human antibody. In some embodiments, disclosed herein is a multi-specific binding molecule comprising a first binding domain and a second binding domain, wherein the first binding domain comprises the anti-CDH17 antibody or antigen-binding fragment thereof. In some embodiments, disclosed herein is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, disclosed herein is a nucleic acid encoding the anti-CDH17 antibody or antigen-binding fragment thereof disclosed herein. In some embodiments, disclosed herein is a nucleic acid encoding the multi-specific binding molecule disclosed herein. In some embodiments, disclosed herein is a nucleic acid encoding a protein disclosed herein. In some embodiments, the nucleic acid comprises DNA, RNA, SSRNA, siRNA, microRNA, or mRNA. In some embodiments, an expression vector comprises a nucleic acid disclosed herein. In some embodiments, a host cell comprises an expression vector or a nucleic acid disclosed herein. In some embodiments, a pharmaceutical composition comprises the anti-CDH17 antibody or antigen-binding fragment thereof, the multi-specific binding molecule, the protein, the conjugate, the nucleic acid, the expression vector or the host cell and a pharmaceutically acceptable carrier or excipient. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is a protein comprising the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having g an amino acid sequence LLSWXIGNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX₇AX₈SVRX (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the cytotoxic agent is MMAE. In some embodiments, the protein further comprises a linker. In some embodiments, the linker comprises a bond, a cleavable linker, and/or a non-cleavable linker. In some embodiments, the linker comprises an attachment group, a protease-cleavable linker, and/or a spacer. In some embodiments, the attachment group comprises maleimide and/or caproic acid. In some embodiments, the protease-cleavable linker is a cathepsin-cleavable linker. In some embodiments, the cathepsin-cleavable linker is a valine-citrulline linker. In some embodiments, the protein further comprises a spacer. In some embodiments, the spacer is PABC. In some embodiments, the protein has a drug antibody ratio (DAR) of about 2, 4, or 8. In some embodiments, the DAR is about 4. In some embodiments, the Fc domain is derived from IgA, IgD, IgE, IgG, or IgM. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is a conjugate comprising Formula (I): Ab-(L-D)n wherein Ab comprises an anti-CDH17 antibody or antigen-binding fragment thereof, wherein anti-CDH17 antibody or antigen-binding fragment thereof comprising (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 43-60, and (c) a HCDR3 having at least 80% sequence identity to the amino acid sequence set out in any one of SEQ ID NOs: 61-75; L is a linker; D is a cytotoxic agent; and n is a drug antibody ratio, wherein the drug antibody ratio is from about 1 to about 8. In some embodiments, the anti-CDH17 antibody or antigen-binding fragment thereof comprises (a) a HCDR1 having an amino acid sequence as set out in SEQ ID NO: 41, (b) a HCDR2 having an amino acid sequence as set out in SEQ ID NO: 47, and (c) a HCDR3 having an amino acid sequence as set out in SEQ ID NO: 72. In some embodiments, the anti-CDH17 antibody or antigen-binding fragment thereof comprises an amino acid sequence having at least 80% sequence identity to the amino acid sequence set out in any one of SEQ ID NO: 2-40 and 109. In some embodiments, the anti-CDH17 antibody or antigen-binding fragment thereof comprises an amino acid sequence having an amino acid sequence as set out in SEQ ID NO: 109. In some embodiments, the anti-CDH17 antibody or antigen-binding fragment thereof further comprises a Fc domain. In some embodiments, the Fc domain is derived from IgA, IgD, IgE, IgG, or IgM. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the cytotoxic agent is MMAE. In some embodiments, the linker comprises an attachment group, a protease-cleavable linker, and/or a spacer. In some embodiments, the attachment group comprises maleimide and/or caproic acid. In some embodiments, the protease-cleavable linker is a cathepsin-cleavable linker. In some embodiments, the cathepsin-cleavable linker is a valine-citrulline linker. In some embodiments, the conjugate further comprises a spacer. In some embodiments, the spacer is PABC. In some embodiments, n is about 4. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the anti-CDH17 antibody or antigen-binding fragment thereof, the multi-specific binding molecule, the conjugate, the expression vector, the host cell, the method, or the pharmaceutical composition is for use in a therapy. In some embodiments, the anti-CDH17 antibody or antigen-binding fragment thereof, the multi-specific binding molecule, the conjugate, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, use of the anti-CDH17 antibody or antigen-binding fragment thereof, the multi-specific binding molecule, the protein, the conjugate, the nucleic acid, the expression vector, the host cell, or the pharmaceutical composition in the manufacture of a medicament for treatment of cancer. In some embodiments, the cancer is gastric, pancreatic, neuroendocrine, or colorectal cancer. In some embodiments, use of the anti-CDH17 antibody or antigen-binding fragment thereof, the multi-specific binding molecule, the protein, the conjugate, the nucleic acid, the expression vector, the host cell, or the pharmaceutical composition, for diagnosis of cancer. In some embodiments, the cancer is gastric, pancreatic, neuroendocrine, or colorectal cancer.

Provided herein is a nucleic acid encoding an anti-CDH17 antibody or antigen-binding fragment thereof, a multi-specific binding molecule described herein, a protein disclosed herein, or a conjugate described herein. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX, (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ a HCDR2 ID NO: 41); (b) having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, Xu is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X, is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX 19 GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, use of the anti-CDH17 antibody or antigen-binding fragment thereof, the multi-specific binding molecule, the protein, the conjugate, the nucleic acid, the expression vector, the host cell, or the pharmaceutical composition in the manufacture of a medicament for treatment of cancer. In some embodiments, the cancer is gastric, pancreatic, neuroendocrine, or colorectal cancer. In some embodiments, use of the anti-CDH17 antibody or antigen-binding fragment thereof, the multi-specific binding molecule, the protein, the conjugate, the nucleic acid, the expression vector, the host cell, or the pharmaceutical composition, for diagnosis of cancer. In some embodiments, the cancer is gastric, pancreatic, neuroendocrine, or colorectal cancer.

Provided herein is an expression vector comprising a nucleic acid encoding an anti-CDH17 antibody or antigen-binding fragment thereof disclosed herein, a multi-specific binding molecule disclosed herein, or a protein disclosed herein. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ a HCDR2 ID NO: 41); (b) having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is a host cell comprising a nucleic acid or an expression vector encoding an anti-CDH17 antibody or antigen-binding fragment thereof disclosed herein, a multi-specific binding molecule disclosed herein, or a protein disclosed herein. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) having an amino acid sequence a HCDR2 LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is a method of producing an anti-CDH17 antibody or antigen-binding fragment thereof disclosed herein, a multi-specific binding molecule disclosed herein, or a protein disclosed herein, the method comprising maintaining the host cell in a medium to produce the anti-CDH17 antibody or antigen-binding fragment thereof, the multi-specific binding molecule, the protein and isolating or purifying the anti-CDH17 antibody or antigen-binding fragment thereof, the multi-specific binding molecule, the protein produced by the host cell. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX₇AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ 41); (b) a HCDR2 ID NO: having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX 19 GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWXIGNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX: SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₅ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is a pharmaceutical composition for treating cancer, comprising an anti-CDH17 antibody or antigen-binding fragment thereof disclosed herein, a-specific binding molecule disclosed herein, a protein disclosed herein, a nucleic acid disclosed herein, an expression vector or a host cell disclosed herein and a pharmaceutically acceptable carrier or excipient. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX, (SEQ ID NO: 86), wherein X₆ is H or Y, X₇ is G, T, V, K or N, and X, is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, Xu is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ 41); (b) a HCDR2 ID NO: having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX 19 GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ a HCDR2 ID NO: 41); (b) having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the pharmaceutical composition further comprises at least one additional therapeutic agent. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition is for diagnosis of cancer. In some embodiments, the cancer is gastric, pancreatic, neuroendocrine, or colorectal cancer.

Provided herein is a method for treating cancer in a subject in need thereof comprising administering to the subject a therapeutically effective amount of an anti-CDH17 antibody or antigen-binding fragment thereof disclosed herein, a multi-specific binding molecule disclosed herein, a protein disclosed herein, a nucleic acid disclosed herein, an expression vector disclosed herein, a host cell disclosed herein, or a pharmaceutical composition disclosed herein. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) having a HCDR2 an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX 19 GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X, is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX 19 GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ a HCDR2 ID NO: 41); (b) having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the pharmaceutical composition further comprises at least one additional therapeutic agent. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

Provided herein is use of an anti-CDH17 antibody or antigen-binding fragment thereof disclosed herein, a multi-specific binding molecule disclosed herein, a protein disclosed herein, ta nucleic acid disclosed herein, an expression vector disclosed herein, a host cell disclosed herein, or a pharmaceutical composition disclosed herein in the manufacture of a medicament for treatment of cancer. In some embodiments, the cancer is gastric, pancreatic, neuroendocrine, or colorectal cancer. In some embodiments, use of the anti-CDH17 antibody or antigen-binding fragment thereof, the multi-specific binding molecule, the protein, the conjugate, the nucleic acid, the expression vector, the host cell, or the pharmaceutical composition, for diagnosis of cancer. In some embodiments, the cancer is gastric, pancreatic, neuroendocrine, or colorectal cancer. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOS: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX, (SEQ ID NO: 85), wherein X₇ is G, T, V, K or N, X₈ is R or K, and X, is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₅ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X, is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX, (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the pharmaceutical composition further comprises at least one additional therapeutic agent. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy

Provided herein is a kit comprising an anti-CDH17 antibody or antigen-binding fragment thereof disclosed herein, a multi-specific binding molecule disclosed herein, a protein disclosed herein, a nucleic acid disclosed herein, an expression vector disclosed herein, a host cell disclosed herein, or a pharmaceutical composition disclosed herein, and instructions for use. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX₇AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX₇AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX, (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, Xu is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof comprises an amino acid sequence having at least 90% sequence identity to the amino acid sequence set out in any one of SEQ ID NO: 2-40 and 109. In some embodiments, the anti-CDH17 antibody or antigen-binding fragment thereof comprises an amino acid sequence having the amino acid sequence set out in any one of SEQ ID NO: 2-40 and 109. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 3-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the multi-specific binding molecule comprises a first binding domain and a second binding domain, wherein the first binding domain comprises the anti-CDH17 antibody or antigen-binding fragment thereof. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX: SVRX, (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX₉ (SEQ ID NO: 85), wherein X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ a HCDR2 ID NO: 41); (b) having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the first binding domain and/or the second binding domain comprises a single chain variable fragment (scFv), a Fab fragment, or a combination thereof. In some embodiments, the first binding domain binds to an antigen expressed on the surface of a cancer cell. In some embodiments, the second binding domain binds to an antigen expressed on the surface of an immune cell. In some embodiments, the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134. In some embodiments, the multi-specific binding molecule further comprises a Fc domain. In some embodiments, the Fc domain is derived from IgA, IgD, IgE, IgG, or IgM. In some embodiments, the multi-specific binding molecule comprises a linker peptide. In some embodiments, the linker peptide comprises about 2-20 amino acids. In some embodiments, the linker peptide comprises (G₄S)ₙ, (SG₄)ₙ, G₄(SG₄)ₙ or G₂(SG₂)ₙ, wherein n is selected from 1 to 10. In some embodiments, the linker peptide comprises GGGGSGGGGS (SEQ ID NO: 100) or GGGGS (SEQ ID NO: 101). In some embodiments, the linker peptide links the anti-CDH17 antibody or antigen-binding fragment thereof to an Fc domain. In some embodiments, the protein comprises the anti-CDH17 antibody or antigen-binding fragment thereof conjugated to a cytotoxic agent, and/or a Fc domain. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75. In some embodiments, the anti-CDH17 antibody or antigen-binding fragment thereof comprises (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75. In some embodiments, the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75. In some embodiments, the anti-CDH17 antibody or antigen-binding fragment thereof has at least 90% sequence identity to an amino acid sequence set out in SEQ ID NO: 1. In some embodiments, the anti-CDH17 antibody or antigen-binding fragment thereof has at least 95% sequence identity to an amino acid sequence set out in SEQ ID NO: 1. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 62. In some embodiments, the anti-CDH17 antibody or antigen-binding fragment thereof has at least 80% sequence identity to the amino acid sequence set out in SEQ ID NO: 2. In some embodiments, the anti-CDH17 antibody or antigen-binding fragment thereof comprises a HCDR1 as set out in SEQ ID NO: 41, a HCDR2 as set out in SEQ ID NO: 44, and/or a HCDR3 as set out in SEQ ID NO: 63. In some embodiments, the anti-CDH17 antibody or antigen-binding fragment thereof has at least 80% sequence identity to the amino acid sequence set out in SEQ ID NO: 3. In some embodiments, the anti-CDH17 antibody or antigen-binding fragment thereof comprises a HCDR1 as set out in SEQ ID NO: 41, a HCDR2 as set out in SEQ ID NO: 43, and/or a HCDR3 as set out in SEQ ID NO: 64. In some embodiments, the anti-CDH17 antibody or antigen-binding fragment thereof has at least 80% sequence identity to the amino acid sequence set out in SEQ ID NO: 4. In some embodiments, the anti-CDH17 antibody or antigen-binding fragment thereof comprises a HCDR1 as set out in SEQ ID NO: 41, a HCDR2 as set out in SEQ ID NO: 45, and/or a HCDR3 as set out in SEQ ID NO: 65. the anti-CDH17 antibody or antigen-binding fragment thereof has at least 80% sequence identity to the amino acid sequence set out in SEQ ID NO: 5. In some embodiments, the anti-CDH17 antibody or antigen-binding fragment thereof comprises a HCDR1 as set out in SEQ ID NO: 41, a HCDR2 as set out in SEQ ID NO: 46, and/or a HCDR3 as set out in SEQ ID NO: 65. In some embodiments, the anti-CDH17 antibody or antigen-binding fragment thereof has at least 80% sequence identity to the amino acid sequence set out in SEQ ID NO: 6. In some embodiments, the anti-CDH17 antibody or antigen-binding fragment thereof comprises at least one amino acid substitutions at position 25-106 in an amino acid sequence as set out in SEQ ID NO: 1. In some embodiments, the at least one amino acid substitutions comprise at least two, three, four, five, six, or seven amino acid substitutions. In some embodiments, the at least one amino acid substitutions comprise a substitution of an amino acid with I, Y, D, R, N, S, A, E, T, F, H, V, L, or K. In some embodiments, the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof, comprises (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX: SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX, (SEQ ID NO: 85), wherein X₇ is G, T, V, K or N, X₈ is R or K, and X, is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX₇AX₈SVRL (SEQ ID NO: 87), wherein X₆ is H or Y, X₇ is G, T, V, K or N, and X₈ is R or K. In some embodiments, the anti-CDH17 antibody or antigen-binding fragment thereof comprises a T67V mutation in an amino acid sequence as set out in SEQ ID NO: 1. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂YX₁₄EYX₁₅X₁₆X₁₇VX₁₈G (SEQ ID NO: 92), wherein X₁₀ is S or D, X₁₁ is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₁₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) having an amino a HCDR2 acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A. In some embodiments, the anti-CDH17 antibody or antigen-binding fragment thereof comprises (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A. In some embodiments, the anti-CDH17 antibody or antigen-binding fragment thereof comprises Q37Y, A38Y, R52I and/or T67V in an amino acid sequence as set out in SEQ ID NO: 1. In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is at least 5-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 10-fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least 15-fold greater than the binding affinity of an amino acid sequence as set out in SEQ ID NO: 1 to CDH17. In some embodiments, the anti-CDH17 antibody or antigen-binding fragment thereof has a KD for binding to CDH17 of less than 1.0×10⁻⁸ M. In some embodiments, the anti-CDH17 antibody or antigen-binding fragment thereof has a KD for binding to CDH17 of less than 5.0×10⁻⁹ M. In some embodiments, the anti-CDH17 antibody or antigen-binding fragment thereof has a KD for binding to CDH17 of less than 3.0×10⁻⁹ M. In some embodiments, the anti-CDH17 antibody or antigen-binding fragment thereof inhibits CDH17 activity with an IC₅₀ of less than 1.0×10⁻⁹ M. In some embodiments, the anti-CDH17 antibody or antigen-binding fragment thereof inhibits CDH17 activity with an IC₅₀ of less than 100.0×10⁻¹² M. In some embodiments, the anti-CDH17 antibody or antigen-binding fragment thereof inhibits CDH17 activity with an IC₅₀ of less than 50.0×10⁻¹² M. In some embodiments, the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17. In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm of less than 70° C. In some embodiments, the anti-CDH17 antibody or antigen-binding fragment thereof has a Tm of less than 60° C. In some embodiments, the anti-CDH17 antibody or antigen-binding fragment thereof has a Tm of less than 50° C. In some embodiments, the anti-CDH17 antibody or antigen-binding fragment thereof has a higher purity compared to a purity of an amino acid sequence as set out in SEQ ID NO: 1. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%. In some embodiments, the anti-CDH17 antibody or antigen-binding fragment thereof has a purity of 100%. In some embodiments, purity is measured via capillary gel electrophoresis using sodium dodecyl sulfate (CE-SDS). In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB). In some embodiments, the protein has a drug antibody ratio of about 2, 4, or 8. In some embodiments, the Fc domain is derived from IgA, IgD, IgE, IgG, or IgM. In some embodiments, the nucleic acid comprises DNA, RNA, ssRNA, siRNA, microRNA, or mRNA. In some embodiments, the pharmaceutical composition further comprises at least one additional therapeutic agent. In some embodiments, the anti-CDH17 antibody or antigen-binding fragment thereof, the multi-specific binding molecule, the expression vector, the host cell, the method, or the pharmaceutical composition is for use in a therapy. In some embodiments, the anti-CDH17 antibody or antigen-binding fragment thereof, the multi-specific binding molecule, the expression vector, the host cell, the method, or the pharmaceutical composition is for use in a therapy, wherein the therapy is an antibody monotherapy, antibody-drug conjugate (ADC) therapy, T cell-engaging immunotherapy, or CAR-T therapy. In some embodiments, the anti-CDH17 antibody or antigen-binding fragment thereof, the multi-specific binding molecule, the protein, the conjugate, the nucleic acid, the expression vector, the host cell, or the pharmaceutical composition is for use in the manufacture of a medicament for treatment of cancer. In some embodiments, the cancer is gastric, pancreatic, neuroendocrine, or colorectal cancer. In some embodiments, the anti-CDH17 antibody or antigen-binding fragment thereof, the multi-specific binding molecule, the protein, the conjugate, the nucleic acid, the expression vector, the host cell, or the pharmaceutical composition, is for diagnosis of cancer. In some embodiments, the cancer is gastric, pancreatic, neuroendocrine, or colorectal cancer.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable” refers to approved or approvable by a regulatory agency of the Federal or a state government or listed in the U.S. Pharmacopeia (U.S.P.) or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent. A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

As used herein, “non-canonical” amino acids can refer to amino acid residues in D- or L-form that are not among the 20 canonical amino acids generally incorporated into naturally occurring proteins.

Unless otherwise specified, a “nucleotide sequence encoding a protein” includes all nucleotide sequences that are degenerate versions of each other and thus encode the same amino acid sequence.

The term “optional” or “optionally” denotes that a subsequently described event or circumstance can but need not occur, and that the description includes instances where the event or circumstance occurs and instances in which it does not.

The term “exogenous” refers to any material introduced from or originating from outside a cell, a tissue or an organism that is not produced by or does not originate from the same cell, tissue, or organism in which it is being introduced.

The term “transduced”, “transfected”, or “transformed” refers to a process by which exogenous nucleic acid is introduced or transferred into a cell. A “transduced,” “transfected,” or “transformed” cell (e.g., a mammalian cell, a hepatocyte) is one that has been transduced, transfected or transformed with exogenous nucleic acid (e.g., an expression vector) that includes an exogenous nucleic acid encoding a polypeptide).

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

The term “antigen-binding domain” is used to refer to one or more antibody variable domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

Unless otherwise specified, a “nucleotide sequence encoding a protein” includes all nucleotide sequences that are degenerate versions of each other and thus encode the same amino acid sequence.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

The term “antibody” refers to a protein with an immunoglobulin fold that specifically binds to an antigen via its variable region or regions. The term “antibody” is used herein in the broadest sense and encompasses monoclonal antibodies, polyclonal antibodies, dimers, multimers, multispecific antibodies (e.g., bispecific antibodies), and antibody fragments, so long as they exhibit the desired biological activity (Miller et al., J. Immunol. 170:4854-4861, 2003). Antibodies may be murine, human, humanized, chimeric, or derived from other species. (Janeway, C., Travers, P., Walport, M., Shlomchik (2001) Immunol. Biology, 5th Ed., Garland Publishing, New York). A target antigen generally has numerous binding sites, also called epitopes, recognized by CDRs (complementarity determining regions) on multiple antibodies. Each antibody that specifically binds to a different epitope has a different structure. Thus, one antigen may have more than one corresponding antibody. An antibody can, e.g., include a full-length immunoglobulin molecule or an immunologically active portion of a full-length immunoglobulin molecule, i.e., a molecule that contains an antigen-binding site that immunospecifically binds an antigen of a target of interest or part thereof. The immunoglobulin disclosed herein can be of any type (e.g., IgG, IgE, IgM, IgD, and IgA), class (e.g., IgG1, IgG2, IgG3, IgG4, IgAQ1 and IgA2) or subclass of immunoglobulin molecule. The immunoglobulins can be derived from any species. In one aspect, however, the immunoglobulin is of human, murine, or rabbit origin.

Antibodies bound to various types of molecules, such as polyethylene glycols (PEGs), may be used as modified antibodies. Methods for modifying antibodies are already established in the art.

The term “antigen-binding fragments” refers to a portion of a full-length antibody or a polypeptide that includes a portion of a full-length antibody, that retains antigen-binding activity via its variable region or regions. Examples of antigen-binding fragments include Fab, Fab′, F(ab′)2, and Fv fragments; diabodies; linear antibodies; minibodies (Olafsen et al., Protein Eng. Design & Sel. 17(4):315-323, 2004), fragments produced by a Fab expression library, single-chain antibody molecules; and multispecific antibodies formed from antigen-binding fragments.

The term “complementarity determining region” or “CDR” refers to one of the three hypervariable regions (or HVRs) that are known to confer (at least in part) antigen-binding specificity in each antibody light chain and each antibody heavy. The three CDRs in the antibody heavy chain and the antibody light chain interrupt four framework regions in the heavy chain variable domain and the light chain variable domain. The CDRs of each chain are typically referred to as CDR1, CDR2, and CDR3, numbered sequentially starting from the N-terminus, and are also typically identified by the chain in which the particular CDR is located.

The “framework regions” or “FRs” of different light immunoglobulin chains and different heavy immunoglobulin chains are relatively conserved within different antibodies produced by a mammal. The framework regions of light and heavy immunoglobulin chains serve to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

“Monospecific” antigen-binding protein refers to the ability of the antigen-binding protein, such as an antibody, to bind only one epitope. “Bispecific” antigen-binding protein refers to the ability of the antigen-binding protein to bind two different epitopes. “Multispecific” antigen-binding protein refers to the ability of the antigen-binding protein to bind more than one epitope. In certain embodiments, a multispecific antigen-binding protein, such as a multispecific antibody, encompasses a bispecific antigen-binding protein or a bispecific antibody. For bispecific and multispecific antigen-binding proteins provided herein, the epitopes can be on the same antigen, or each epitope can be on a different antigen. Therefore, in certain embodiments, a multispecific antigen-binding protein provided herein, such as a bispecific antibody, binds to two different antigens. In certain embodiments, the multispecific antigen-binding protein, such as a bispecific antibody, binds to different epitopes on one antigen. In certain embodiments, a multispecific antigen-binding protein provided herein binds to each epitope with a dissociation constant (Kd) of about ≤1 M, about ≤100 nM, about ≤10 nM, about ≤1 nM, about <0.1 nM, about <0.01 nM, or about <0.001 nM (e.g., about 10⁻⁸ M or less, e.g., from about 10⁻⁸ M to about 10⁻¹³M, e.g., from about 10⁻⁹ M to about 10⁻¹⁰ M).

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala or A); arginine (Arg or R); asparagine (Asn or N); aspartic acid (Asp or D); cysteine (Cys or C); glutamine (GIn or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V).

The term “administer” refers to a method of delivering compositions to the desired site of biological action. These methods include, but are not limited to, topical delivery, parenteral delivery, intravenous delivery, intradermal delivery, subcutaneous delivery, intramuscular delivery, colonic delivery, rectal delivery, or intraperitoneal delivery.

The term “promoter” means a DNA sequence recognized by enzymes/proteins in a cell (e.g., a mammalian cell, a hepatocyte) required to initiate the transcription of an operably linked coding sequence (e.g., a nucleic acid encoding a polypeptide (e.g., any of the exemplary polypeptides described herein). A promoter typically refers, to e.g. a nucleotide sequence to which an RNA polymerase and/or any associated factor binds and at which transcription is initiated. The promoter can be constitutive, inducible, or tissue-specific (e.g., a liver-specific promoter).

The term “enhancer” refers to a nucleotide sequence that can increase the transcription of an operably linked nucleic acid (e.g., a nucleic acid encoding a polypeptide (e.g., any of the exemplary polypeptides described herein). An enhancer can increase the level of transcription by providing additional binding sites for transcription-associated proteins (e.g., transcription factors). Unlike promoters, enhancers can act at distances further away from the transcription start site (e.g., as compared to a promoter).

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 or Megalign. The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The term “affinity” refers to the strength of the sum of all non-covalent interactions between an antigen-binding site and its antigen. Unless otherwise indicated, “affinity” refers to intrinsic binding affinity, which reflects a 1:1 interaction between an antigen-binding domain and an antigen. Affinity can be measured, e.g., using surface plasmon resonance (SPR) technology (e.g., BIACORE®) or biolayer interferometry (e.g., FORTEBIO®). Additional methods for determining the affinity of an antigen-binding domain and its antigen are known in the art.

The term “single-chain polypeptide” means a polypeptide comprising a single polypeptide chain.

The term “multi-chain polypeptide” means a complex of two or more (e.g., 2, 3, 4, 5, 6, 7, or 8) polypeptide chains (e.g., the same or different polypeptide chains) that covalently and/or non-covalently associate with each other. For example, two or more polypeptide chains of a multi-chain polypeptide can associate through the use of two domains that associate with each other (e.g., two Fc domains or IL-15 and the sushi domain of IL-15 receptor alpha).

The section headings used herein are for organizational purposes only and are not to be construed as limiting the subject matter described.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. The Kabat numbering of residues may be determined for a given antibody by alignment at regions of homology of the sequence of the antibody with a “standard” Kabat numbered sequence. It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342:877-883.

Other numbering conventions for CDR sequences available to a skilled person include “AbM” (University of Bath) and “contact” (university College London) methods. The minimum overlapping region using at least two of the Kabat, Chothia, AbM and contact methods can be determined to provide the ‘minimum binding unit’. The minimum binding unit may be a sub-portion of a CDR. Table 1 below represents one definition using each numbering convention for each CDR or binding unit. The Kabat numbering scheme is used in Table 1 to number variable domain amino acid sequence. It should be noted that some of the CDR definition may vary depending on the individual publication used.

TABLE 1

| | Kabat | Chothia | AbM | Contact | Minimum |
|---|---|---|---|---|---|
| | CDR | CDR | CDR | CDR | Binding Unit |
| H1 | 31-35/35A/35B | 26-32/33/34 | 26-35/35A/35B | 30-35/35A/35B | 31-32 |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

The terms “residue,” “moiety” or “group” refers to a component that is covalently bound or linked to another component. For example, a “moiety of a cytotoxic agent” refers to a cytotoxic agent that is covalently linked to one or more groups such as a Linker (L2), which itself can be optionally further linked to an antibody via linker (L1).

The terms “payload,” “drug payload,” “therapeutic molecule,” therapeutic payload”, “therapeutic agents,” “therapeutic moieties,” as used interchangeably herein, refers to a chemical or biological moiety that is conjugated to an antibody or antigen binding fragment (e.g., an anti-CDH17 antibody or antigen binding fragment disclosed herein), and can include any therapeutic or diagnostic agent, for example, but not limited to, cytotoxic agents, both for cancer and for non-cancer indications; a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB).

### CDRs Modification & Lead Sequences

The anti-CDH17 antibody or antigen-binding fragment thereof may have at least one or more amino acid modifications in various regions of the antibody. The anti-CDH17 antibody or antigen-binding fragment thereof may be modified by at least one or more amino acid substitution, deletion, addition, or a combination thereof at one or more position while the variant CDH17 binding protein substantially retains the biological characteristics of the unmodified protein, such as binding to CDH17. In some embodiments, the amino acid sequence of the CDH17 antibody or antigen-binding fragment thereof may have at least 1, at least 2, at least 3, at least 4, at least 5, at least 6, at least 7, at least 8, at least 9, at least 10, at least 11, at least 12, at least 13, at least 14, or at least 15 amino acid insertions, deletions, substitutions or a combination thereof.

In some instances, one or more amino acid modifications described herein can be in the antigen-binding (Fab) fragments of an antibody or antibody binding fragment thereof. In some instances, the modification can be within the Fab region of a heavy chain polypeptide (H) and a first light chain polypeptide (L). In some embodiments, the modification can be within a heavy chain variable domain (VH), a heavy chain constant domain (CH1), a light chain variable domain (VL), a light chain constant domain (CL), or a combination thereof. In some embodiments, one or more amino acid modification can be in the Fab VH and VL with the N-terminal region. The VH, VL, HC or LC sequence disclosed herein may be a variant sequence with up to 15 amino acid substitutions, insertion, or deletions. For example, the variant sequence may have up to 15, 14, 13, 12, 11, 10, 9, 8, 7, 6, 5, 4, 3, 2, or 1 amino acid substitution(s), addition(s), or deletion(s). The sequence variation may exclude one or more or all of the CDRs, for example, the CDRs are the same as the VH, VL, HC or LC sequence and the variation is in the remaining portion of the VH or VL, HC, or LC sequence, so that the CDR sequences are fixed and intact.

Each of HCDR1, HCDR2, HCDR3, LCDR1, LCDR2, and LCDR3 may be modified alone or in combination with any other CDR, in any permutation or combination. Typically, the modification is a substitution, particularly a conserved substitution, for example in Table 2 below.

TABLE 2

| Side Chain | Members |
|---|---|
| Hydrophobic | Met, Ala, Val, Leu Ile |
| Neutral Hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

In some embodiments, the anti-CDH17 antibody or an antigen-binding fragment thereof may comprise CDRs: HCDR1 selected from the group consisting of SEQ ID NO: 41, HCDR2 selected from the group consisting of SEQ ID NOs: 42-60, and HCDR3 selected from the group consisting of SEQ ID NOs: 61-78, functional fragments thereof, functional variants thereof, and combination thereof.

In some embodiments, the anti-CDH17 antibody or an antigen-binding fragment thereof may comprise CDRs: HCDR1 of SEQ ID NO: 41, HCDR2 of SEQ ID NOS: 43, and/or HCDR3 of SEQ ID NO: 62, functional fragments thereof, functional variants thereof, and combination thereof. In some embodiments, the anti-CDH17 antibody or an antigen-binding fragment thereof may comprise CDRs: HCDR1 of SEQ ID NO: 41, HCDR2 of SEQ ID NOs: 44, and/or HCDR3 of SEQ ID NO: 63, functional fragments thereof, functional variants thereof, and combination thereof. In some embodiments, the anti-CDH17 antibody or an antigen-binding fragment thereof may comprise CDRs: HCDR1 of SEQ ID NO: 41, HCDR2 of SEQ ID NOs: 43, and/or HCDR3 of SEQ ID NO: 64, functional fragments thereof, functional variants thereof, and combination thereof. In some embodiments, the anti-CDH17 antibody or an antigen-binding fragment thereof may comprise CDRs: HCDR1 of SEQ ID NO: 41, HCDR2 of SEQ ID NOs: 45, and/or HCDR3 of SEQ ID NO: 65, functional fragments thereof, functional variants thereof, and combination thereof. In some embodiments, the anti-CDH17 antibody or an antigen-binding fragment thereof may comprise CDRs: HCDR1 of SEQ ID NO: 41, HCDR2 of SEQ ID NOs: 46, and/or HCDR3 of SEQ ID NO: 65, functional fragments thereof, functional variants thereof, and combination thereof. In some embodiments, the anti-CDH17 antibody or an antigen-binding fragment thereof may comprise CDRs: HCDR1 of SEQ ID NO: 41, HCDR2 of SEQ ID NOs: 47, and/or HCDR3 of SEQ ID NO: 72, functional fragments thereof, functional variants thereof, and combination thereof.

In some embodiments, each domain of three complementarity-determining region LCDR: LCDR1, LCDR2, and LCDR3 for VL, and HCDR-1, HCDR-2, and HCDR-3 for VH may be modified alone or in combination with any other CDR, in any permutation or combination. In some embodiments, the LCDR-1, LCDR-2, LCDR-3, HCDR-1, HCDR2, and HCDR-3 may be modified by substitution, deletion or insertion of 1 amino acid to 10 amino acids. In some embodiments, the LCDR-1, LCDR-2, LCDR-3, HCDR-1, HCDR2, and HCDR-3 may have 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 1 amino acid to 9 amino acids, 1 amino acid to 10 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 2 amino acids to 9 amino acids, 2 amino acids to 10 amino acids, 3 amino acids to 4 amino acids, 3 amino to 5 amino acids, 3 amino acids to 6 amino acids, 3 amino acids to 7 amino acids, 3 amino acids to 8 amino acids, 3 amino acids to 9 amino acids, 3 amino acids to 10 amino acids, 4 amino acids to 5 amino acids, 4 amino acids to 6 amino acids, 4 amino acids to 7 amino acids, 4 amino acids to 8 amino acids, 4 amino acids to 9 amino acids, 4 amino acids to 10 amino acids, 5 amino acids to 6 amino acids, 5 amino acids to 7 amino acids, 5 amino acids to 8 amino acids, 5 amino acids to 9 amino acids, 5 amino acids to 10 amino acids, 6 amino acids to 7 amino acids, 6 amino acids to 8 amino acids, 6 amino acids to 9 amino acids, 6 amino acids to 10 amino acids, 7 amino acids to 8 amino acids, 7 amino acids to 9 amino acids, 7 amino acids to 10 amino acids, 8 amino acids to 9 amino acids, 8 amino acids to 10 amino acids, or 9 amino acids to 10 amino acids modifications. In some embodiments, the LCDR-1, LCDR-2, LCDR-3, HCDR-1, HCDR2, and HCDR-3 may have 1 amino acid, 2 amino acids, 3 amino acids, 4 amino acids, 5 amino acids, 6 amino acids, 7 amino acids, 8 amino acids, 9 amino acids, or 10 amino acids modifications. In some embodiments, the LCDR-1, LCDR-2, LCDR-3, HCDR-1, HCDR2, and HCDR-3 may have at least 1 amino acid, 2 amino acids, 3 amino acids, 4 amino acids, 5 amino acids, 6 amino acids, 7 amino acids, 8 amino acids, or 9 amino acids modifications. In some embodiments, the LCDR-1, LCDR-2, LCDR-3, HCDR-1, HCDR2, and HCDR-3 may have at most 2 amino acids, 3 amino acids, 4 amino acids, 5 amino acids, 6 amino acids, 7 amino acids, 8 amino acids, 9 amino acids, or 10 amino acids modifications.

In some embodiments, the anti-CDH17 antibodies or antigen-binding fragment thereof may comprise HCDR1 selected from the group consisting of SEQ ID NO: 41 or a variant HCDR1 thereof. In some embodiments, the HCDR1 may include a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to SEQ ID NO: 41.

In some embodiments, the anti-CDH17 antibodies or antigen-binding fragment thereof may comprise HCDR2 selected from the group consisting of SEQ ID NOs: 43-60 or a variant HCDR2 thereof. In some embodiments, the polypeptide may comprise a HCDR2 having the amino acid sequence set out in any one of SEQ ID NOs: 42-60. In some embodiments, the HCDR2 includes a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 42-60.

In some embodiments, the anti-CDH17 antibodies or antigen-binding fragment thereof may comprise HCDR3 selected from the group consisting of SEQ ID NOs: 61-75 or a variant HCDR3 thereof. In some embodiments, the polypeptide may comprise a HCDR3 having the amino acid sequence set out in any one of SEQ ID NOs: 62-75. In some embodiments, the anti-CDH17 antibody or antigen-binding fragment thereof may include a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 61-75.

Exemplary anti-CDH17 antibodies or antigen-binding fragment thereof sequences are listed in Table 3 below.

TABLE 3

| | | SEQ ID | |
|---|---|---|---|
| Construct | Description | NO: | Sequence |
| CDH17_ | Full Length | 1 | QVQLQESGGGLVQPGGSTRLSCVSSRTFSYY |
| vhh_0001 | Sequence | | DMGWFRQAPGKEREFVALLSWNGENAEYSD |
| | | | SVMGRFTVSRGNTQNSVNLQMNNLKPEDTGI |
| | | | YYCAVTHGGARSVRSWGQGTQVTVSS |
| | HCDR1 | 41 | YDMG |
| | HCDR2 | 42 | LLSWNGENAEYSDSVMG |
| | HCDR3 | 61 | THGGARSVRS |
| CDH17_ | Full Length | 2 | QVQLQESGGGLVQPGGSTRLSCVSSRTFSYY |
| vhh_0951 | Sequence | | DMGWFRQAPGKEREFVALLSWRGNNAEYSD |
| | | | DVMGRFTVSRGNTQNSVNLQMNNLKPEDTG |
| | | | IYYCAVTYGTARSVRLWGQGTQVTVSS |
| | HCDR1 | 41 | YDMG |
| | HCDR2 | 43 | LLSWRGNNAEYSDDVMG |
| | HCDR3 | 62 | TYGTARSVRL |
| CDH17_ | Full Length | 3 | QVQLQESGGGLVQPGGSTRLSCVSSRTFSYY |
| vhh_0947 | Sequence | | DMGWFRQAPGKEREFVALLSWRGNNAEYR |
| | | | DSVMGRFTVSRGNTQNSVNLQMNNLKPEDT |
| | | | GIYYCAVTYGVAKSVRLWGQGTQVTVSS |
| | HCDR1 | 41 | YDMG |
| | HCDR2 | 44 | LLSWRGNNAEYRDSVMG |
| | HCDR3 | 63 | TYGVAKSVRL |
| CDH17_ | Full Length | 4 | QVQLQESGGGLVQPGGSTRLSCVSSRTFSYY |
| vhh_0949 | Sequence | | DMGWFRQAPGKEREFVALLSWRGNNAEYSD |
| | | | DVMGRFTVSRGNTQNSVNLQMNNLKPEDTG |
| | | | IYYCAVTYGKARSVRLWGQGTQVTVSS |
| | HCDR1 | 41 | YDMG |
| | HCDR2 | 43 | LLSWRGNNAEYSDDVMG |
| | HCDR3 | 64 | TYGKARSVRL |
| CDH17_ | Full Length | 5 | QVQLQESGGGLVQPGGSTRLSCVSSRTFSYY |
| vhh_0946 | Sequence | | DMGWFRQAPGKEREFVALLSWRGNNAEYSD |
| | | | FVMGRFTVSRGNTQNSVNLQMNNLKPEDTGI |
| | | | YYCAVTYGVARSVRLWGQGTQVTVSS |
| | HCDR1 | 41 | YDMG |
| | HCDR2 | 45 | LLSWRGNNAEYSDFVMG |
| | HCDR3 | 65 | TYGVARSVRL |
| CDH17_ | Full Length | 6 | QVQLQESGGGLVQPGGSTRLSCVSSRTFSYY |
| vhh_0945 | Sequence | | DMGWFRQAPGKEREFVALLSWRGNNAEYSD |
| | | | SVHGRFTVSRGNTQNSVNLQMNNLKPEDTGI |
| | | | YYCAVTYGVARSVRLWGQGTQVTVSS |
| | HCDR1 | 41 | YDMG |
| | HCDR2 | 46 | LLSWRGNNAEYSDSVHG |
| | HCDR3 | 65 | TYGVARSVRL |
| CDH17_ | Full Length | 7 | QVQLQESGGGLVQPGGSTRLSCVSSRTFSYY |
| vhh_0648 | Sequence | | DMGWFRQAPGKEREFVALLSWRGNNAEYSD |
| | | | SVMGRFVVSRGNTQNSVNLQMNNLKPEDTG |
| | | | IYYCAVTYGVAKSVRSWGQGTQVTVSS |
| | HCDR1 | 41 | YDMG |
| | HCDR2 | 47 | LLSWRGNNAEYSDSVMG |
| | HCDR3 | 66 | TYGVAKSVRS |
| CDH17_ | Full Length | 8 | QVQLQESGGGLVQPGGSTRLSCVSSRTFSYY |
| vhh_0650 | Sequence | | DMGWFRQAPGKEREFVALLSWRGNNAEYR |
| | | | DSVMGRFTVSRGNTQNSVNLQMNNLKPEDT |
| | | | GIYYCAVTYGGAKSVRLWGQGTQVTVSS |
| | HCDR1 | 41 | YDMG |
| | HCDR2 | 44 | LLSWRGNNAEYRDSVMG |
| | HCDR3 | 67 | TYGGAKSVRL |
| CDH17_ | Full Length | 9 | QVQLQESGGGLVQPGGSTRLSCVSSRTFSYY |
| vhh_0646 | Sequence | | DMGWFRQAPGKEREFVALLSWRGNNAEYR |
| | | | DSVMGRFTVSRGNTQNSVNLQMNNLKPEDT |
| | | | GIYYCAVTYGGARSVRLWGQGTQVTVSS |
| | HCDR1 | 41 | YDMG |
| | HCDR2 | 44 | LLSWRGNNAEYRDSVMG |
| | HCDR3 | 68 | TYGGARSVRL |
| CDH17_ | Full Length | 10 | QVQLQESGGGLVQPGGSTRLSCVSSRTFSYY |
| vhh_0952 | Sequence | | DMGWFRQAPGKEREFVALLSWRGNNAEYSD |
| | | | DVMGRFTVSRGNTQNSVNLQMNNLKPEDTG |
| | | | IYYCAVTYGNARSVRLWGQGTQVTVSS |
| | HCDR1 | 41 | YDMG |
| | HCDR2 | 43 | LLSWRGNNAEYSDDVMG |
| | HCDR3 | 69 | TYGNARSVRL |
| CDH17_ | Full Length | 11 | QVQLQESGGGLVQPGGSTRLSCVSSRTFSYY |
| vhh_0677 | Sequence | | DMGWFRQAPGKEREFVALLSWRGNNAEYSD |
| | | | DVMGRFTVSRGNTQNSVNLQMNNLKPEDTG |
| | | | IYYCAVTYGKARSVRSWGQGTQVTVSS |
| | HCDR1 | 41 | YDMG |
| | HCDR2 | 43 | LLSWRGNNAEYSDDVMG |
| | HCDR3 | 70 | TYGKARSVRS |
| CDH17_ | Full Length | 12 | QVQLQESGGGLVQPGGSTRLSCVSSRTFSYY |
| vhh_0271 | Sequence | | DMGWFRQAPGKEREFVALLSWRGNNAEYSD |
| | | | SVMGRFVVSRGNTQNSVNLQMNNLKPEDTG |
| | | | IYYCAVTYGVARSVRSWGQGTQVTVSS |
| | HCDR1 | 41 | YDMG |
| | HCDR2 | 47 | LLSWRGNNAEYSDSVMG |
| | HCDR3 | 71 | TYGVARSVRS |
| CDH17_ | Full Length | 13 | QVQLQESGGGLVQPGGSTRLSCVSSITFSYYD |
| vhh_0622 | Sequence | | MGWFRQAPGKEREFVALLSWRGENAEYSDS |
| | | | VMGRFTVSRGNTQNSVNLQMNNLKPEDTGI |
| | | | YYCAVTYGGARSVRSWGQGTQVTVSS |
| | HCDR1 | 41 | YDMG |
| | HCDR2 | 48 | LLSWRGENAEYSDSVMG |
| | HCDR3 | 72 | TYGGARSVRS |
| CDH17_ | Full Length | 14 | QVQLQESGGGLVQPGGSTRLSCVSSRTFSYY |
| vhh_0653 | Sequence | | DMGWFRQAPGKEREFVALLSWRGINAEYSD |
| | | | TVMGRFVVSRGNTQNSVNLQMNNLKPEDTG |
| | | | IYYCAVTYGVARSVRSWGQGTQVTVSS |
| | HCDR1 | 41 | YDMG |
| | HCDR2 | 49 | LLSWRGINAEYSDTVMG |
| | HCDR3 | 71 | TYGVARSVRS |
| CDH17_ | Full Length | 15 | QVQLQESGGGLVQPGGSTRLSCVSSRTFSYY |
| vhh_0591 | Sequence | | DMGWFRQAPGKEREFVALLSWRGNNAEYSD |
| | | | SVMGRFTVSRGNTQNSVNLQMNNLKPEDTGI |
| | | | YYCAVTYGGARSVRLWGQGTQVTVSS |
| | HCDR1 | 41 | YDMG |
| | HCDR2 | 47 | LLSWRGNNAEYSDSVMG |
| | HCDR3 | 68 | TYGGARSVRL |
| CDH17_ | Full Length | 16 | QVQLQESGGGLVQPGGSTRLSCVSSRTFSYY |
| vhh_0644 | Sequence | | DMGWFRQAPGKEREFVALLSWRGNNAEYR |
| | | | DSVMGRFVVSRGNTQNSVNLQMNNLKPEDT |
| | | | GIYYCAVTYGVARSVRSWGQGTQVTVSS |
| | HCDR1 | 41 | YDMG |
| | HCDR2 | 44 | LLSWRGNNAEYRDSVMG |
| | HCDR3 | 71 | TYGVARSVRS |
| CDH17_ | Full Length | 17 | QVQLQESGGGLVQPGGSTRLSCVSSRTFSYY |
| vhh_0652 | Sequence | | DMGWFRQYPGKEREFVALLSWRGNYAEYSD |
| | | | SVMGRFVVSRGNTQNSVNLQMNNLKPEDTG |
| | | | IYYCAVTYGGARSVRLWGQGTQVTVSS |
| | HCDR1 | 41 | YDMG |
| | HCDR2 | 50 | LLSWRGNYAEYSDSVMG |
| | HCDR3 | 68 | TYGGARSVRL |
| CDH17_ | Full Length | 18 | QVQLQESGGGLVQPGGSTRLSCVSSRTFSYY |
| vhh_0651 | Sequence | | DMGWFRQYPGKEREFVALLSWRGNNAEYSD |
| | | | SVMGRFVVSRGNTQNSVNLQMNNLKPEDTG |
| | | | IYYCAVTLGVARSVRSWGQGTQVTVSS |
| | HCDR1 | 41 | YDMG |
| | HCDR2 | 47 | LLSWRGNNAEYSDSVMG |
| | HCDR3 | 73 | TLGVARSVRS |
| CDH17_ | Full Length | 19 | QVQLQESGGGLVQPGGSTRLSCVSSRTFSYY |
| vhh_0672 | Sequence | | DMGWFRQAPGKEREFVALLSWRGNNAEYSD |
| | | | SVHGRFTVSRGNTQNSVNLQMNNLKPEDTGI |
| | | | YYCAVTYGGARSVRLWGQGTQVTVSS |
| | HCDR1 | 41 | YDMG |
| | HCDR2 | 46 | LLSWRGNNAEYSDSVHG |
| | HCDR3 | 68 | TYGGARSVRL |
| CDH17_ | Full Length | 20 | QVQLQESGGGLVQPGGSTRLSCVSSRTFSYY |
| vhh_0674 | Sequence | | DMGWFRQAPGKEREFVALLSWRGNNAEYSD |
| | | | TVMGRFTVSRGNTQNSVNLQMNNLKPEDTG |
| | | | IYYCAVTYGGARSVRKWGQGTQVTVSS |
| | HCDR1 | 41 | YDMG |
| | HCDR2 | 51 | LLSWRGNNAEYSDTVMG |
| | HCDR3 | 74 | TYGGARSVRK |
| CDH17_ | Full Length | 21 | QVQLQESGGGLVQPGGSTRLSCVSSRTFSYY |
| vhh_0950 | Sequence | | DMGWFRQAPGKEREFVALLSWRGNNSEYSD |
| | | | SVHGRFTVSRGNTQNSVNLQMNNLKPEDTGI |
| | | | YYCAVTYGGARSVRLWGQGTQVTVSS |
| | HCDR1 | 41 | YDMG |
| | HCDR2 | 52 | LLSWRGNNSEYSDSVHG |
| | HCDR3 | 68 | TYGGARSVRL |
| CDH17_ | Full Length | 22 | QVQLQESGGGLVQPGGSTRLSCVSSRTFSYY |
| vhh_0675 | Sequence | | DMGWFRQAPGKEREFVALLSWRGNNAEYSD |
| | | | TVMGRFTVSRGNTQNSVNLQMNNLKPEDTG |
| | | | IYYCAVTYGGARSVRAWGQGTQVTVSS |
| | HCDR1 | 41 | YDMG |
| | HCDR2 | 51 | LLSWRGNNAEYSDTVMG |
| | HCDR3 | 75 | TYGGARSVRA |
| CDH17_ | Full Length | 23 | QVQLQESGGGLVQPGGSTRLSCVSSRTFSYY |
| vhh_0263 | Sequence | | DMGWFRQAPGKEREFVALLSWRGNYAEYSD |
| | | | SVMGRFVVSRGNTQNSVNLQMNNLKPEDTG |
| | | | IYYCAVTYGGARSVRSWGQGTQVTVSS |
| | HCDR1 | 41 | YDMG |
| | HCDR2 | 50 | LLSWRGNYAEYSDSVMG |
| | HCDR3 | 72 | TYGGARSVRS |
| CDH17_ | Full Length | 24 | QVQLQESGGGLVQPGGSTRLSCVSSRTFSYY |
| vhh_0948 | Sequence | | DMGWFRQAPGKEREFVALLSWRGNNAEYSD |
| | | | DVHGRFTVSRGNTQNSVNLQMNNLKPEDTGI |
| | | | YYCAVTYGGARSVRLWGQGTQVTVSS |
| | HCDR1 | 41 | YDMG |
| | HCDR2 | 53 | LLSWRGNNAEYSDDVHG |
| | HCDR3 | 68 | TYGGARSVRL |
| CDH17_ | Full Length | 25 | QVQLQESGGGLVQPGGSTRLSCVSSRTFSYY |
| vhh_0274 | Sequence | | DMGWFRQAPGKEREFVALLSWRGNNAEYSD |
| | | | SVMGRFVVSRGNTQNSVNLQMNNLKPEDTG |
| | | | IYYCAVTYGGAKSVRSWGQGTQVTVSS |
| | HCDR1 | 41 | YDMG |
| | HCDR2 | 47 | LLSWRGNNAEYSDSVMG |
| | HCDR3 | 76 | TYGGAKSVRS |
| CDH17_ | Full Length | 26 | QVQLQESGGGLVQPGGSTRLSCVSSRTFSYY |
| vhh_0676 | Sequence | | DMGWFRQAPGKEREFVALLSWRGNNAEYSD |
| | | | DVMGRFTVSRGNTQNSVNLQMNNLKPEDTG |
| | | | IYYCAVTYGTARSVRSWGQGTQVTVSS |
| | HCDR1 | 41 | YDMG |
| | HCDR2 | 43 | LLSWRGNNAEYSDDVMG |
| | HCDR3 | 77 | TYGTARSVRS |
| CDH17_ | Full Length | 27 | QVQLQESGGGLVQPGGSTRLSCVSSRTFSYY |
| vhh_0670 | Sequence | | DMGWFRQAPGKEREFVALLDWRGNYAEYS |
| | | | DSVMGRFTVSRGNTQNSVNLQMNNLKPEDT |
| | | | GIYYCAVTYGGARSVRSWGQGTQVTVSS |
| | HCDR1 | 41 | YDMG |
| | HCDR2 | 54 | LLDWRGNYAEYSDSVMG |
| | HCDR3 | 72 | TYGGARSVRS |
| CDH17_ | Full Length | 28 | QVQLQESGGGLVQPGGSTRLSCVSSRTFSYY |
| vhh_0259 | Sequence | | DMGWFRQYPGKEREFVALLSWRGNNAEYSD |
| | | | SVMGRFTVSRGNTQNSVNLQMNNLKPEDTGI |
| | | | YYCAVTYGGARSVRSWGQGTQVTVSS |
| | HCDR1 | 41 | YDMG |
| | HCDR2 | 47 | LLSWRGNNAEYSDSVMG |
| | HCDR3 | 72 | TYGGARSVRS |
| CDH17_ | Full Length | 29 | QVQLQESGGGLVQPGGSTRLSCVSSRTFSYY |
| vhh_0372 | Sequence | | DMGWFRQAPGKEREFVALLDWRGNNAEYS |
| | | | DSVMGRFTVSRGNTQNSVNLQMNNLKPEDT |
| | | | GIYYCAVTYGGARSVRSWGQGTQVTVSS |
| | HCDR1 | 41 | YDMG |
| | HCDR2 | 55 | LLDWRGNNAEYSDSVMG |
| | HCDR3 | 72 | TYGGARSVRS |
| CDH17_ | Full Length | 30 | QVQLQESGGGLVQPGGSTRLSCVSSRTFSYY |
| vhh_0613 | Sequence | | DMGWFRQAPGKEREFVALLSWRGENAEYSD |
| | | | SVMGRFTVSRGNTQNSVNLQMNNLKPEDTGI |
| | | | YYCAVTYGGARSVRSWGQGTQVTVSS |
| | HCDR1 | 41 | YDMG |
| | HCDR2 | 48 | LLSWRGENAEYSDSVMG |
| | HCDR3 | 72 | TYGGARSVRS |
| CDH17_ | Full Length | 31 | QVQLQESGGGLVQPGGSTRLSCVSSRTFSYY |
| vhh_0632 | Sequence | | DMGWFRQAPGKEREFVALLSWRGERAEYSD |
| | | | SVMGRFTVSRGNTQNSVNLQMNNLKPEDTGI |
| | | | YYCAVTYGGARSVRSWGQGTQVTVSS |
| | HCDR1 | 41 | YDMG |
| | HCDR2 | 56 | LLSWRGERAEYSDSVMG |
| | HCDR3 | 72 | TYGGARSVRS |
| CDH17_ | Full Length | 32 | QVQLQESGGGLVQPGGSTRLSCVSSRTFSYY |
| vhh_0689 | Sequence | | DMGWFRQAPGKEREFVALLSWRGNNAEYSD |
| | | | FVMGRFTVSRGNTQNSVNLQMNNLKPEDTGI |
| | | | YYCAVTYGGARSVRLWGQGTQVTVSS |
| | HCDR1 | 41 | YDMG |
| | HCDR2 | 45 | LLSWRGNNAEYSDFVMG |
| | HCDR3 | 68 | TYGGARSVRL |
| CDH17_ | Full Length | 33 | QVQLQESGGGLVQPGGSTRLSCVSSRTFSYY |
| vhh_0173 | Sequence | | DMGWFRQAPGKEREFVALLSWRGNNAEYR |
| | | | DSVMGRFTVSRGNTQNSVNLQMNNLKPEDT |
| | | | GIYYCAVTYGGARSVRSWGQGTQVTVSS |
| | HCDR1 | 41 | YDMG |
| | HCDR2 | 44 | LLSWRGNNAEYRDSVMG |
| | HCDR3 | 72 | TYGGARSVRS |
| CDH17_ | Full Length | 34 | QVQLQESGGGLVQPGGSTRLSCVSSRTFSYY |
| vhh_0629 | Sequence | | DMGWFRQAPGKEREFVALLDWRGENAEYS |
| | | | DSVMGRFTVSRGNTQNSVNLQMNNLKPEDT |
| | | | GIYYCAVTYGGARSVRSWGQGTQVTVSS |
| | HCDR1 | 41 | YDMG |
| | HCDR2 | 57 | LLDWRGENAEYSDSVMG |
| | HCDR3 | 72 | TYGGARSVRS |
| CDH17 | Full Length | 35 | QVQLQESGGGLVQPGGSTRLSCVSSRTFSYY |
| vhh_0267 | Sequence | | DMGWFRYAPGKEREFVALLSWRGNNAEYA |
| | | | DSVMGRFTVSRGNTQNSVNLQMNNLKPEDT |
| | | | GIYYCAVTYGGARSVRSWGQGTQVTVSS |
| | HCDR1 | 41 | YDMG |
| | HCDR2 | 58 | LLSWRGNNAEYADSVMG |
| | HCDR3 | 72 | TYGGARSVRS |
| CDH17_ | Full Length | 36 | QVQLQESGGGLVQPGGSTRLSCVSSRTFSYY |
| vhh_0587 | Sequence | | DMGWFRQAPGKEREFVALLSWRGINAEYSD |
| | | | SVMGRFTVSRGNTQNSVNLQMNNLKPEDTGI |
| | | | YYCAVTYGGARSVRSWGQGTQVTVSS |
| | HCDR1 | 41 | YDMG |
| | HCDR2 | 59 | LLSWRGINAEYSDSVMG |
| | HCDR3 | 72 | TYGGARSVRS |
| CDH17_ | Full Length | 37 | QVQLQESGGGLVQPGGSTRLSCVSSRTFSYY |
| vhh_0262 | Sequence | | DMGWFRQAPGKEREFVALLSWRGNNAEYSD |
| | | | TVMGRFTVSRGNTQNSVNLQMNNLKPEDTG |
| | | | IYYCAVTYGGARSVRSWGQGTQVTVSS |
| | HCDR1 | 41 | YDMG |
| | HCDR2 | 51 | LLSWRGNNAEYSDTVMG |
| | HCDR3 | 72 | TYGGARSVRS |
| CDH17_ | Full Length | 38 | QVQLQESGGGLVQPGGSTRLSCVSSRTFSYY |
| vhh_0645 | Sequence | | DMGWFRQYPGKEREFVALLSWRGNYAEYSD |
| | | | SVMGRFVVSRGNTQNSVNLQMNNLKPEDTG |
| | | | IYYCAVTYGGARSVRSWGQGTQVTVSS |
| | HCDR1 | 41 | YDMG |
| | HCDR2 | 50 | LLSWRGNYAEYSDSVMG |
| | HCDR3 | 72 | TYGGARSVRS |
| CDH17_ | Full Length | 39 | QVQLQESGGGLVQPGGSTRLSCVSSRTFSYY |
| vhh_0395 | Sequence | | DMGWFRQAPGKEREFVALLSWRGNNAEYSE |
| | | | SVMGRFTVSRGNTQNSVNLQMNNLKPEDTGI |
| | | | YYCAVTYGGARSVRSWGQGTQVTVSS |
| | HCDR1 | 41 | YDMG |
| | HCDR2 | 60 | LLSWRGNNAEYSESVMG |
| | HCDR3 | 72 | TYGGARSVRS |
| CDH17 | Full Length | 40 | QVQLQESGGGLVQPGGSTRLSCVSSRTFSYY |
| vhh_0688 | Sequence | | DMGWFRQAPGKEREFVALLSWRGNNAEYSD |
| | | | DVMGRFTVSRGNTQNSVNLQMNNLKPEDTG |
| | | | IYYCAVTYGNARSVRSWGQGTQVTVSS |
| | HCDR1 | 41 | YDMG |
| | HCDR2 | 43 | LLSWRGNNAEYSDDVMG |
| | HCDR3 | 78 | TYGNARSVRS |
| CDH17_ | Full Length | 109 | QVQLQESGGGLVQPGGSTRLSCVSSRTFSYY |
| vhh_0156 | Sequence | | DMGWFRQAPGKEREFVALLSWRGNNAEYSD |
| (BHB156) | | | SVMGRFTVSRGNTQNSVNLQMNNLKPEDTGI |
| | | | YYCAVTYGGARSVRSWGQGTQVTVSS |
| | HCDR1 | 41 | YDMG |
| | HCDR2 | 47 | LLSWRGNNAEYSDSVMG |
| | HCDR3 | 72 | TYGGARSVRS |

In some embodiments, the anti-CDH17 antibody or an antigen-binding fragment thereof may comprise HCDR1 having an amino acid sequence of YDMG (SEQ ID NO: 41).

In some embodiments, the anti-CDH17 antibody or an antigen-binding fragment thereof may comprise HCDR2 having an amino acid sequence of LLSWRGX₂NAEYX₃DX₄VX₅G (SEQ ID NO: 79), wherein:
X₂ is be selected from: E or N;
X₃ is be selected from: S or R;
X₄ is be selected from: S, D, or F; and
X₅ is be selected from: M or H.

In some embodiments, the anti-CDH17 antibody or an antigen-binding fragment thereof may comprise HCDR2 having an amino acid sequence of LLSWX₁GNNAEYX₃DX₄VXG (SEQ ID NO: 81), wherein:
X₁ is be selected from: N or R;
X₃ is be selected from: S or R;
X₄ is be selected from: S, D, or F; and
X₅ is be selected from: M or H.

In some embodiments, the anti-CDH17 antibody or an antigen-binding fragment thereof may comprise HCDR2 having an amino acid sequence of LLSWX₁GX₂NAEYRDX₄VX₅G (SEQ ID NO: 82), wherein:
X₁ is be selected from: N or R;
X₂ is be selected from: E or N;
X₄ is be selected from: S, D, or F; and
X₅ is be selected from: M or H.

In some embodiments, the anti-CDH17 antibody or an antigen-binding fragment thereof may comprise HCDR2 having an amino acid sequence of LLSWX₁GX₂NAEYX₃DX₄VX₅G (SEQ ID NO: 83), wherein:
X₁ is be selected from: N or R;
X₂ is be selected from: E or N;
X₃ is be selected from: S or R;
X₄ is be selected from: D or F; and
X₅ is be selected from: M or H.

In some embodiments, the anti-CDH17 antibody or an antigen-binding fragment thereof may comprise HCDR2 having an amino acid sequence of LLSWX₁GX₂NAEYX₃DX₄VHG (SEQ ID NO: 84), wherein:
X₁ is be selected from: N or R;
X₂ is be selected from: E or N;
X₃ is be selected from: S or R; and
X₄ is be selected from: S, D, or F.

In some embodiments, the anti-CDH17 antibody or an antigen-binding fragment thereof may comprise HCDR2 having an amino acid sequence of LLDWX₁₁GX₁₂X₁₃X₁₄EYX₁₅X₁₆X₁₇VX₁₈G (SEQ ID NO: 88), wherein:
X₁₁ is be selected from: N or R;
X₁₂ is be selected from: E, N, I, or Y;
X₁₃ is be selected from: N or Y;
X₁₄ is be selected from: A or S;
X₁₅ is be selected from: S, R, or A;
X₁₆ is be selected from: D or E;
X₁₇ is be selected from: S, D, F or T; and
X₁₈ is be selected from: M or H.

In some embodiments, the anti-CDH17 antibody or an antigen-binding fragment thereof may comprise HCDR2 having an amino acid sequence of LLX₁₀WRGX₁₂X₁₃X₁₄EYX₁₅X₁₆X₁₇VX₁₈G (SEQ ID NO: 90), wherein:
X₁₀ is be selected from: S or D;
X₁₂ is be selected from: E, N, I, or Y;
X₁₃ is be selected from: N or Y;
X₁₄ is be selected from: A or S;
X₁₅ is be selected from: S, R, or A;
X₁₆ is be selected from: D or E;
X₁₇ is be selected from: S, D, F or T; and
X₁₈ is be selected from: M or H.

In some embodiments, the anti-CDH17 antibody or an antigen-binding fragment thereof may comprise HCDR2 having an amino acid sequence of LLX₁₀WX₁₁GX₁₂X₁₃X₁₄EYX₁₅X₁₆X₁₇VX₁₈G (SEQ ID NO: 91), wherein:

X₁₀ is be selected from: S or D;
X₁₁ is be selected from: N or R;
X₁₂ is be selected from: N, I, or Y;
X₁₃ is be selected from: N or Y;
X₁₄ is be selected from: A or S;
X₁₅ is be selected from: S, R, or A;
X₁₆ is be selected from: D or E;
X₁₇ is be selected from: S, D, F or T; and
X₁₈ is be selected from: M or H.

In some embodiments, the anti-CDH17 antibody or an antigen-binding fragment thereof may comprise HCDR2 having an amino acid sequence of LLX₁₀WX₁₁GX₁₂YX₁₄EYX₁₅X₁₆X₁₇VX₁₈G (SEQ ID NO: 92), wherein:
X₁₀ is be selected from: S or D;
X₁₁ is be selected from: N or R;
X₁₂ is be selected from: E, N, I, or Y;
X₁₃ is be selected from: N or Y;
X₁₄ is be selected from: A or S;
X₁₅ is be selected from: S, R, or A;
X₁₆ is be selected from: D or E;
X₁₇ is be selected from: S, D, F or T; and
X₁₈ is be selected from: M or H.

In some embodiments, the anti-CDH17 antibody or an antigen-binding fragment thereof may comprise HCDR2 having an amino acid sequence of LLX₁₀WX₁₁GX₁₂X₁₃SEYX₁₅X₁₆X₁₇VX₁₈G (SEQ ID NO: 93), wherein:
X₁₀ is be selected from: S or D;
X₁₁ is be selected from: N or R;
X₁₂ is be selected from: E, N, I, or Y;
X₁₃ is be selected from: N or Y;
X₁₅ is be selected from: S, R, or A;
X₁₆ is be selected from: D or E;
X₁₇ is be selected from: S, D, F or T; and
X₁₈ is be selected from: M or H.

In some embodiments, the anti-CDH17 antibody or an antigen-binding fragment thereof may comprise HCDR2 having an amino acid sequence of LLX₁₀WX₁₁GX₁₂X₁₃X₁₄EYX₁₅X₁₆X₁₇VX₁₈G (SEQ ID NO: 94), wherein:
X₁₀ is be selected from: S or D;
X₁₁ is be selected from: N or R;
X₁₂ is be selected from: E, N, I, or Y;
X₁₃ is be selected from: N or Y;
X₁₄ is be selected from: A or S;
X₁₅ is be selected from: R or A;
X₁₆ is be selected from: D or E;
X₁₇ is be selected from: S, D, F or T; and
X₁₈ is be selected from: M or H.

In some embodiments, the anti-CDH17 antibody or an antigen-binding fragment thereof may comprise HCDR2 having an amino acid sequence of LLX₁₀WX₁₁GX₁₂X₁₃X₁₄EYX₁₅EX₁₇VX₁₈G (SEQ ID NO: 95), wherein:
X₁₀ is be selected from: S or D;
X₁₁ is be selected from: N or R;
X₁₂ is be selected from: E, N, I, or Y;
X₁₃ is be selected from: N or Y;
X₁₄ is be selected from: A or S;
X₁₅ is be selected from: S, R, or A;
X₁₇ is be selected from: S, D, F or T; and
X₁₈ is be selected from: M or H.

In some embodiments, the anti-CDH17 antibody or an antigen-binding fragment thereof may comprise HCDR2 having an amino acid sequence of LLX₁₀WX₁₁GX₁₂X₁₃X₁₄EYX₁₅X₁₆X₁₇VHG (SEQ ID NO: 97), wherein:
X₁₀ is be selected from: S or D;
X₁₁ is be selected from: N or R;
X₁₂ is be selected from: E, N, I, or Y;
X₁₃ is be selected from: N or Y;
X₁₄ is be selected from: A or S;
X₁₅ is be selected from: S, R, or A;
X₁₆ is be selected from: D or E; and
X₁₇ is be selected from: S, D, F or T.

In some embodiments, the anti-CDH17 antibody or an antigen-binding fragment thereof may comprise HCDR3 having an amino acid sequence of TX₆GX-AX₈SVRX₉ (SEQ ID NO: 80), wherein:
X₆ is H or Y;
X₇ is G, T, V, K or N;
X₈ is R or K; and
X₉ is S or L.

In some embodiments, the anti-CDH17 antibody or an antigen-binding fragment thereof may comprise HCDR3 having an amino acid sequence of TYGX-AX₈SVRX₉ (SEQ ID NO: 85), wherein:
X₇ is G, T, V, K or N;
X₈ is R or K; and
X₉ is S or L.

In some embodiments, the anti-CDH17 antibody or an antigen-binding fragment thereof may comprise HCDR3 having an amino acid sequence of TX₆GX₇AKSVRX₉ (SEQ ID NO: 86), wherein:
X₆ is H or Y;
X₇ is G, T, V, K or N; and
X₉ is S or L.

In some embodiments, the anti-CDH17 antibody or an antigen-binding fragment thereof may comprise HCDR3 having an amino acid sequence of TX₆GX-AX: SVRL (SEQ ID NO: 87), wherein:

X₆ is H or Y;

X₇ is G, T, V, K or N; and

X₈ is R or K.

In some embodiments, the anti-CDH17 antibody or an antigen-binding fragment thereof may comprise HCDR3 having an amino acid sequence of TX₁₉GX₂₀AX₂₁SVRX₂₂ (SEQ ID NO: 96), wherein:
X₁₉ is H or Y;
X₂₀ is G, T, V, K, or N;
X₂₁ is R or K; and
X₂₂ is S, L, K, or A.

In some embodiments, the anti-CDH17 antibody or an antigen-binding fragment thereof may comprise HCDR3 having an amino acid sequence of TYGX₂₀AX₂₁SVRX₂₂ (SEQ ID NO: 98), wherein:
X₂₀ is G, T, V, K, or N;
X₂₁ is R or K; and
X₂₂ is S, L, K, or A.

In some embodiments, the anti-CDH17 antibody or an antigen-binding fragment thereof may comprise HCDR3 having an amino acid sequence of TX₁₉GX₂₀AKSVRX₂₂ (SEQ ID NO: 99), wherein:
X₁₉ is H or Y;
X₂₀ is G, T, V, K, or N; and
X₂₂ is S, L, K, or A.

Exemplary sequences for HCDR2 and HCDR3 are listed in Table 4 below.

TABLE 4

| Sequence | SEQ ID NO: |
|---|---|
| LLSWRGX2NAEYX₃DX₄VX₅G | 79 |
| TX₆GX₇AX₈SVRX₉ | 80 |
| LLSWX₁GNNAEYX₃DX₄VX₅G | 81 |
| LLSWX₁GX₂NAEYRDX₄VX₅G | 82 |
| LLSWX₁GX₂NAEYX₃DX₄VX₅G | 83 |
| LLSWX₁GX₂NAEYX₃DX₄VHG | 84 |
| TYGX₇AX₈SVRX₉ | 85 |
| TX₆GX₇AK SVRX₉ | 86 |
| TX₆GX₇AX₈SVRL | 87 |
| LLDWX₁₁GX₁₂X₁₃X₁₄EYX₁₅X₁₆X₁₇VX₁₈G | 88 |
| TX₁₉GX₂₀AX₂₁SVRX₂₂ | 89 |
| LLX₁₀WRGX₁₂X₁₃X₁₄EYX₁₅X₁₆X₁₇VX₁₈G | 90 |
| LLX₁₀WX₁₁GX₁₂X₁₃X₁₄EYX₁₅X₁₆X₁₇VX₁₈G | 91 |
| LLX₁₀WX₁₁GX₁₂YX₁₄EYX₁₅X₁₆X₁₇VX₁₈G | 92 |
| LLX₁₀WX₁₁GX₁₂X₁₃SEYX₁₅X₁₆X₁₇VX₁₈G | 93 |
| LLX₁₀WX₁₁GX₁₂X₁₃X₁₄EYX₁₅X₁₆X₁₇VX₁₈G | 94 |
| LLX₁₀WX₁₁GX₁₂X₁₃X₁₄EYX₁₅EX₁₇VX₁₈G | 95 |
| TX₁₉GX₂₀AX₂₁SVRX₂₂ | 96 |
| LLX₁₀WX₁₁GX₁₂X₁₃X₁₄EYX₁₅X₁₆X₁₇VHG | 97 |
| TYGX₂₀AX₂₁SVRX₂₂ | 98 |
| TX₁₉GX₂₀AKSVRX₂₂ | 99 |

In some embodiments, the anti-CDH17 antibody or antigen-binding fragment thereof may comprise a T67V mutation in an amino acid sequence as set out in SEQ ID NO: 1.

In some embodiments, the anti-CDH17 antibody or antigen-binding fragment thereof may comprise a Q37Y, A38Y, R52I and/or T67V mutations in an amino acid sequence as set out in SEQ ID NO: 1.

In some embodiments, the anti-CDH17 antibodies or antigen-binding fragment thereof may comprise a heavy chain variable region selected from the group consisting of SEQ ID NOs: 1-40 functional fragments thereof, and functional variants thereof.

In some embodiments, the anti-CDH17 antibodies or antigen-binding fragment thereof may comprise a heavy chain variable region of SEQ ID NO: 1. In some embodiments, the anti-CDH17 antibodies or antigen-binding fragment thereof may comprise a heavy chain variable region of SEQ ID NO: 2. In some embodiments, the anti-CDH17 antibodies or antigen-binding fragment thereof may comprise a heavy chain variable region of SEQ ID NO: 3. In some embodiments, the anti-CDH17 antibodies or antigen-binding fragment thereof may comprise a heavy chain variable region of SEQ ID NO: 4. In some embodiments, the anti-CDH17 antibodies or antigen-binding fragment thereof may comprise a heavy chain variable region of SEQ ID NO: 5. In some embodiments, the anti-CDH17 antibodies or antigen-binding fragment thereof may comprise a heavy chain variable region of SEQ ID NO: 6. In some embodiments, the anti-CDH17 antibodies or antigen-binding fragment thereof may comprise a heavy chain variable region of SEQ ID NO: 109.

In some embodiments, a heavy chain variable domain described herein may be modified by at least one amino acid substitution, deletion or insertion, wherein the variant anti-CDH17 antibodies or antigen-binding fragment thereof substantially retains the biological characteristics of the unmodified protein, such as binding to CDH17. In some embodiments, a heavy chain variable domain may be modified alone or in combination with any other CDR, in any permutation or combination. In some embodiments, the heavy chain variable domain may be modified by the substitution, deletion or insertion of 1 amino acid to 10 amino acids. In some embodiments, the heavy chain variable domain may have 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 1 amino acid to 9 amino acids, 1 amino acid to 10 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 2 amino acids to 9 amino acids, 2 amino acids to 10 amino acids, 3 amino acids to 4 amino acids, 3 amino to 5 amino acids, 3 amino acids to 6 amino acids, 3 amino acids to 7 amino acids, 3 amino acids to 8 amino acids, 3 amino acids to 9 amino acids, 3 amino acids to 10 amino acids, 4 amino acids to 5 amino acids, 4 amino acids to 6 amino acids, 4 amino acids to 7 amino acids, 4 amino acids to 8 amino acids, 4 amino acids to 9 amino acids, 4 amino acids to 10 amino acids, 5 amino acids to 6 amino acids, 5 amino acids to 7 amino acids, 5 amino acids to 8 amino acids, 5 amino acids to 9 amino acids, 5 amino acids to 10 amino acids, 6 amino acids to 7 amino acids, 6 amino acids to 8 amino acids, 6 amino acids to 9 amino acids, 6 amino acids to 10 amino acids, 7 amino acids to 8 amino acids, 7 amino acids to 9 amino acids, 7 amino acids to 10 amino acids, 8 amino acids to 9 amino acids, 8 amino acids to 10 amino acids, or 9 amino acids to 10 amino acids modifications. In some embodiments, HCDR may have 1 amino acid, 2 amino acids, 3 amino acids, 4 amino acids, 5 amino acids, 6 amino acids, 7 amino acids, 8 amino acids, 9 amino acids, or 10 amino acids modifications. In some embodiments, the heavy chain variable domain may have at least 1 amino acid, 2 amino acids, 3 amino acids, 4 amino acids, 5 amino acids, 6 amino acids, 7 amino acids, 8 amino acids, or 9 amino acids modification. In some embodiments, the heavy chain variable domain may have at most 2 amino acids, 3 amino acids, 4 amino acids, 5 amino acids, 6 amino acids, 7 amino acids, 8 amino acids, 9 amino acids, or 10 amino acids modification.

In some embodiments, the anti-CDH17 antibodies or antigen-binding fragment thereof may comprise a heavy chain variable domain selected from the group consisting of SEQ ID NOs: 1-40 or a variant heavy chain variable domain thereof. In some embodiments, the anti-CDH17 antibody or antigen-binding fragment thereof may include a HCDR sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 1-40 or 109.

In some embodiments, the anti-CDH17 antibodies or antigen-binding fragment thereof can comprise SEQ ID NO: 1. The anti-CDH17 antibodies or antigen-binding fragment thereof described herein may be modified by at least one amino acid substitution, deletion or insertion, wherein the variant anti-CDH17 antibodies or antigen-binding fragment thereof substantially retains the biological characteristics of the unmodified protein, such as binding to CDH17. In some embodiments, the anti-CDH17 antibodies or antigen-binding fragment thereof may be modified by the substitution, deletion or insertion of 1 amino acid to 10 amino acids. In some embodiments, the anti-CDH17 antibodies or antigen-binding fragment thereof may have 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 1 amino acid to 9 amino acids, 1 amino acid to 10 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 2 amino acids to 9 amino acids, 2 amino acids to 10 amino acids, 3 amino acids to 4 amino acids, 3 amino to 5 amino acids, 3 amino acids to 6 amino acids, 3 amino acids to 7 amino acids, 3 amino acids to 8 amino acids, 3 amino acids to 9 amino acids, 3 amino acids to 10 amino acids, 4 amino acids to 5 amino acids, 4 amino acids to 6 amino acids, 4 amino acids to 7 amino acids, 4 amino acids to 8 amino acids, 4 amino acids to 9 amino acids, 4 amino acids to 10 amino acids, 5 amino acids to 6 amino acids, 5 amino acids to 7 amino acids, 5 amino acids to 8 amino acids, 5 amino acids to 9 amino acids, 5 amino acids to 10 amino acids, 6 amino acids to 7 amino acids, 6 amino acids to 8 amino acids, 6 amino acids to 9 amino acids, 6 amino acids to 10 amino acids, 7 amino acids to 8 amino acids, 7 amino acids to 9 amino acids, 7 amino acids to 10 amino acids, 8 amino acids to 9 amino acids, 8 amino acids to 10 amino acids, or 9 amino acids to 10 amino acids modifications. In some embodiments, the anti-CDH17 antibodies or antigen-binding fragment thereof may have 1 amino acid, 2 amino acids, 3 amino acids, 4 amino acids, 5 amino acids, 6 amino acids, 7 amino acids, 8 amino acids, 9 amino acids, or 10 amino acids modifications. In some embodiments, the anti-CDH17 antibodies or antigen-binding fragment thereof may have at least 1 amino acid, 2 amino acids, 3 amino acids, 4 amino acids, 5 amino acids, 6 amino acids, 7 amino acids, 8 amino acids, or 9 amino acids modification. In some embodiments, the anti-CDH17 antibodies or antigen-binding fragment thereof may have at most 2 amino acids, 3 amino acids, 4 amino acids, 5 amino acids, 6 amino acids, 7 amino acids, 8 amino acids, 9 amino acids, or 10 amino acids modification.

In some embodiments, the anti-CDH17 antibodies or antigen-binding fragment thereof as disclosed herein can comprise an amino acid sequence, and the amino acid sequence of the polypeptide may be at least 60%, 65%, 70%, 75%, 80%, 85%, 90%, 91%, 92%, 93%, 94%, 95%, 96%, 97%, 98%, 99%, or 100% sequence identity to SEQ ID NO: 1. For example, the amino acid sequence of the anti-CDH17 antibodies or antigen-binding fragment thereof can be between 80% and about 100% identical to the polypeptide sequence of SEQ ID NO: 1.

### Amino Acid Substitution

The HCDR1, HCDR2, HCDR3 can exhibit one of a finite number of main chain conformation (canonicals). The particular canonical structure class of a CDR can be defined by both the length of the CDR and by the loop packing, determined by residues located at key position in both the CDRs and the framework regions. One or more changes in canonical structures by adding, deleting, substituting one or more amino acid within the key position may result in distinct changes in the antibody (e.g., antigen sensitivity or binding affinity).

In some embodiments, the anti-CDH17 antibodies or antigen-binding fragment thereof may comprise at least one, at least two, at least three, at least four, at least five, at least six, at least seven, at least eight, at least nine, at least ten amino acid substitutions at position 25-106 (according to Kabat numbering) in a heavy chain variable domain of SEQ ID NO: 1. In some cases, the substitution of amino acid may be with I, Y, D, R, N, S, A, E, T, F, H, V, L, or K. In some embodiments, the substitution at position at position 25-106 (according to Kabat numbering) in heavy chain variable domain may comprise or any combination thereof R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A. In some embodiments, the anti-CDH17 antibody or antigen-binding fragment thereof may comprise one or more amino acid mutations selected from Q37Y, A38Y, R52I and/or T67V in a heavy chain variable domain of SEQ ID NO: 1.

### Antibody Properties

In some embodiments, the anti-CDH17 antibody or antigen-binding fragment thereof has an increased binding affinity to CDH17 compared to a binding affinity of an amino acid sequence as set out in SEQ ID NO: 1 to CDH17. In some embodiments, the binding affinity of the anti-CDH17 antibody or antigen-binding fragment thereof to CDH17 is about 5-fold to about 15-fold greater than the binding affinity of an amino acid sequence as set out in SEQ ID NO: 1 to CDH17. In some embodiments, the binding affinity of the anti-CDH17 antibody or antigen-binding fragment thereof to CDH17 is about 2 fold to about 5 fold, about 3 fold to about 5 fold, about 4 fold to about 5 fold, about 5 fold to about 6 fold, about 5 fold to about 7 fold, about 5 fold to about 8 fold, about 5 fold to about 9 fold, about 5 fold to about 10 fold, about 5 fold to about 11 fold, about 5 fold to about 12 fold, about 5 fold to about 13 fold, about 5 fold to about 14 fold, about 5 fold to about 15 fold, about 7 fold to about 8 fold, about 7 fold to about 9 fold, about 7 fold to about 10 fold, about 7 fold to about 11 fold, about 7 fold to about 12 fold, about 7 fold to about 13 fold, about 7 fold to about 14 fold, about 7 fold to about 15 fold, about 10 fold to about 11 fold, about 10 fold to about 12 fold, about 10 fold to about 13 fold, about 10 fold to about 14 fold, or about 10 fold to about 15 fold greater than the binding affinity of an amino acid sequence as set out in SEQ ID NO: 1 to CDH17. In some embodiments, the binding affinity of the anti-CDH17 antibody or antigen-binding fragment thereof to CDH17 is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, or about 20 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, or about 20 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at most about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, or about 20 fold greater than the binding affinity of an amino acid sequence as set out in SEQ ID NO: 1 to CDH17.

In some embodiments, the binding affinity of the anti-CDH17 antibody or antigen-binding fragment thereof to CDH17 is about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.6 fold, about 2.7 fold, about 2.8 fold, about 2.9 fold, about 3.0 fold, about 3.1 fold, about 3.2 fold, about 3.3 fold, about 3.4 fold, about 3.5 fold, about 3.6 fold, about 3.7 fold, about 3.8 fold, about 3.9 fold, about 4.0 fold, about 4.1 fold, about 4.2 fold, about 4.3 fold, about 4.4 fold, about 4.5 fold, about 4.6 fold, about 4.7 fold, about 4.8 fold, about 4.9 fold, or about 5.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.6 fold, about 2.7 fold, about 2.8 fold, about 2.9 fold, about 3.0 fold, about 3.1 fold, about 3.2 fold, about 3.3 fold, about 3.4 fold, about 3.5 fold, about 3.6 fold, about 3.7 fold, about 3.8 fold, about 3.9 fold, about 4.0 fold, about 4.1 fold, about 4.2 fold, about 4.3 fold, about 4.4 fold, about 4.5 fold, about 4.6 fold, about 4.7 fold, about 4.8 fold, about 4.9 fold, or about 5.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at most about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.6 fold, about 2.7 fold, about 2.8 fold, about 2.9 fold, about 3.0 fold, about 3.1 fold, about 3.2 fold, about 3.3 fold, about 3.4 fold, about 3.5 fold, about 3.6 fold, about 3.7 fold, about 3.8 fold, about 3.9 fold, about 4.0 fold, about 4.1 fold, about 4.2 fold, about 4.3 fold, about 4.4 fold, about 4.5 fold, about 4.6 fold, about 4.7 fold, about 4.8 fold, about 4.9 fold, or about 5.0 fold greater than the binding affinity of an amino acid sequence as set out in SEQ ID NO: 1 to CDH17.

In some embodiments, the binding affinity of the anti-CDH17 antibody or antigen-binding fragment thereof to CDH17 is about 5.1 fold, about 5.2 fold, about 5.3 fold, about 5.4 fold, about 5.5 fold, about 5.6 fold, about 5.7 fold, about 5.8 fold, about 5.9 fold, about 6.0 fold, about 6.1 fold, about 6.2 fold, about 6.3 fold, about 6.4 fold, about 6.5 fold, about 6.6 fold, about 6.7 fold, about 6.8 fold, about 6.9 fold, about 7.0 fold, about 7.1 fold, about 7.2 fold, about 7.3 fold, about 7.4 fold, about 7.5 fold, about 7.6 fold, about 7.7 fold, about 7.8 fold, about 7.9 fold, about 8.0 fold, about 8.1 fold, about 8.2 fold, about 8.3 fold, about 8.4 fold, about 8.5 fold, about 8.6 fold, about 8.7 fold, about 8.8 fold, about 8.9 fold, about 9.0 fold, about 9.1 fold, about 9.2 fold, about 9.3 fold, about 9.4 fold, about 9.5 fold, about 9.6 fold, about 9.7 fold, about 9.8 fold, about 9.9 fold or about 10.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least about 5.1 fold, about 5.2 fold, about 5.3 fold, about 5.4 fold, about 5.5 fold, about 5.6 fold, about 5.7 fold, about 5.8 fold, about 5.9 fold, about 6.0 fold, about 6.1 fold, about 6.2 fold, about 6.3 fold, about 6.4 fold, about 6.5 fold, about 6.6 fold, about 6.7 fold, about 6.8 fold, about 6.9 fold, about 7.0 fold, about 7.1 fold, about 7.2 fold, about 7.3 fold, about 7.4 fold, about 7.5 fold, about 7.6 fold, about 7.7 fold, about 7.8 fold, about 7.9 fold, about 8.0 fold, about 8.1 fold, about 8.2 fold, about 8.3 fold, about 8.4 fold, about 8.5 fold, about 8.6 fold, about 8.7 fold, about 8.8 fold, about 8.9 fold, about 9.0 fold, about 9.1 fold, about 9.2 fold, about 9.3 fold, about 9.4 fold, about 9.5 fold, about 9.6 fold, about 9.7 fold, about 9.8 fold, about 9.9 fold or about 10.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at most about 5.1 fold, about 5.2 fold, about 5.3 fold, about 5.4 fold, about 5.5 fold, about 5.6 fold, about 5.7 fold, about 5.8 fold, about 5.9 fold, about 6.0 fold, about 6.1 fold, about 6.2 fold, about 6.3 fold, about 6.4 fold, about 6.5 fold, about 6.6 fold, about 6.7 fold, about 6.8 fold, about 6.9 fold, about 7.0 fold, about 7.1 fold, about 7.2 fold, about 7.3 fold, about 7.4 fold, about 7.5 fold, about 7.6 fold, about 7.7 fold, about 7.8 fold, about 7.9 fold, about 8.0 fold, about 8.1 fold, about 8.2 fold, about 8.3 fold, about 8.4 fold, about 8.5 fold, about 8.6 fold, about 8.7 fold, about 8.8 fold, about 8.9 fold, about 9.0 fold, about 9.1 fold, about 9.2 fold, about 9.3 fold, about 9.4 fold, about 9.5 fold, about 9.6 fold, about 9.7 fold, about 9.8 fold, about 9.9 fold or about 10.0 fold greater than the binding affinity of an amino acid sequence as set out in SEQ ID NO: 1 to CDH17.

In some embodiments, the binding affinity of the anti-CDH17 antibody or antigen-binding fragment thereof to CDH17 is about 10.1 fold, about 10.2 fold, about 10.3 fold, about 10.4 fold, about 10.5 fold, about 10.6 fold, about 10.7 fold, about 10.8 fold, about 10.9 fold, about 11.0 fold, about 11.1 fold, about 11.2 fold, about 11.3 fold, about 11.4 fold, about 11.5 fold, about 11.6 fold, about 11.7 fold, about 11.8 fold, about 11.9 fold, about 12.0 fold, about 12.1 fold, about 12.2 fold, about 12.3 fold, about 12.4 fold, about 12.5 fold, about 12.6 fold, about 12.7 fold, about 12.8 fold, about 12.9 fold, about 13.0 fold, about 13.1 fold, about 13.2 fold, about 13.3 fold, about 13.4 fold, about 13.5 fold, about 13.6 fold, about 13.7 fold, about 13.8 fold, about 13.9 fold, about 14.0 fold, about 14.1 fold, about 14.2 fold, about 14.3 fold, about 14.4 fold, about 14.5 fold, about 14.6 fold, about 14.7 fold, about 14.8 fold, about 14.9 fold, or about 15.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least about 10.1 fold, about 10.2 fold, about 10.3 fold, about 10.4 fold, about 10.5 fold, about 10.6 fold, about 10.7 fold, about 10.8 fold, about 10.9 fold, about 11.0 fold, about 11.1 fold, about 11.2 fold, about 11.3 fold, about 11.4 fold, about 11.5 fold, about 11.6 fold, about 11.7 fold, about 11.8 fold, about 11.9 fold, about 12.0 fold, about 12.1 fold, about 12.2 fold, about 12.3 fold, about 12.4 fold, about 12.5 fold, about 12.6 fold, about 12.7 fold, about 12.8 fold, about 12.9 fold, about 13.0 fold, about 13.1 fold, about 13.2 fold, about 13.3 fold, about 13.4 fold, about 13.5 fold, about 13.6 fold, about 13.7 fold, about 13.8 fold, about 13.9 fold, about 14.0 fold, about 14.1 fold, about 14.2 fold, about 14.3 fold, about 14.4 fold, about 14.5 fold, about 14.6 fold, about 14.7 fold, about 14.8 fold, about 14.9 fold, or about 15.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at most about 10.1 fold, about 10.2 fold, about 10.3 fold, about 10.4 fold, about 10.5 fold, about 10.6 fold, about 10.7 fold, about 10.8 fold, about 10.9 fold, about 11.0 fold, about 11.1 fold, about 11.2 fold, about 11.3 fold, about 11.4 fold, about 11.5 fold, about 11.6 fold, about 11.7 fold, about 11.8 fold, about 11.9 fold, about 12.0 fold, about 12.1 fold, about 12.2 fold, about 12.3 fold, about 12.4 fold, about 12.5 fold, about 12.6 fold, about 12.7 fold, about 12.8 fold, about 12.9 fold, about 13.0 fold, about 13.1 fold, about 13.2 fold, about 13.3 fold, about 13.4 fold, about 13.5 fold, about 13.6 fold, about 13.7 fold, about 13.8 fold, about 13.9 fold, about 14.0 fold, about 14.1 fold, about 14.2 fold, about 14.3 fold, about 14.4 fold, about 14.5 fold, about 14.6 fold, about 14.7 fold, about 14.8 fold, about 14.9 fold, or about 15.0 fold greater than the binding affinity of an amino acid sequence as set out in SEQ ID NO: 1 to CDH17.

In some embodiments, the binding affinity of the anti-CDH17 antibody or antigen-binding fragment thereof to CDH17 is about 15.1 fold, about 15.2 fold, about 15.3 fold, about 15.4 fold, about 15.5 fold, about 15.6 fold, about 15.7 fold, about 15.8 fold, about 15.9 fold, about 16.0 fold, about 16.1 fold, about 16.2 fold, about 16.3 fold, about 16.4 fold, about 16.5 fold, about 16.6 fold, about 16.7 fold, about 16.8 fold, about 16.9 fold, about 17.0 fold, about 17.1 fold, about 17.2 fold, about 17.3 fold, about 17.4 fold, about 17.5 fold, about 17.6 fold, about 17.7 fold, about 17.8 fold, about 17.9 fold, about 18.0 fold, about 18.1 fold, about 18.2 fold, about 18.3 fold, about 18.4 fold, about 18.5 fold, about 18.6 fold, about 18.7 fold, about 18.8 fold, about 18.9 fold, about 19.0 fold, about 19.1 fold, about 19.2 fold, about 19.3 fold, about 19.4 fold, about 19.5 fold, about 19.6 fold, about 19.7 fold, about 19.8 fold, about 19.9 fold or about 20.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at least about 15.1 fold, about 15.2 fold, about 15.3 fold, about 15.4 fold, about 15.5 fold, about 15.6 fold, about 15.7 fold, about 15.8 fold, about 15.9 fold, about 16.0 fold, about 16.1 fold, about 16.2 fold, about 16.3 fold, about 16.4 fold, about 16.5 fold, about 16.6 fold, about 16.7 fold, about 16.8 fold, about 16.9 fold, about 17.0 fold, about 17.1 fold, about 17.2 fold, about 17.3 fold, about 17.4 fold, about 17.5 fold, about 17.6 fold, about 17.7 fold, about 17.8 fold, about 17.9 fold, about 18.0 fold, about 18.1 fold, about 18.2 fold, about 18.3 fold, about 18.4 fold, about 18.5 fold, about 18.6 fold, about 18.7 fold, about 18.8 fold, about 18.9 fold, about 19.0 fold, about 19.1 fold, about 19.2 fold, about 19.3 fold, about 19.4 fold, about 19.5 fold, about 19.6 fold, about 19.7 fold, about 19.8 fold, about 19.9 fold or about 20.0 fold greater than the binding affinity of an amino acid sequence as set out in SEQ ID NO: 1 to CDH17. In some embodiments, the binding affinity of the anti-CDH17 antibody or antigen-binding fragment thereof to CDH17 is at most about 15.1 fold, about 15.2 fold, about 15.3 fold, about 15.4 fold, about 15.5 fold, about 15.6 fold, about 15.7 fold, about 15.8 fold, about 15.9 fold, about 16.0 fold, about 16.1 fold, about 16.2 fold, about 16.3 fold, about 16.4 fold, about 16.5 fold, about 16.6 fold, about 16.7 fold, about 16.8 fold, about 16.9 fold, about 17.0 fold, about 17.1 fold, about 17.2 fold, about 17.3 fold, about 17.4 fold, about 17.5 fold, about 17.6 fold, about 17.7 fold, about 17.8 fold, about 17.9 fold, about 18.0 fold, about 18.1 fold, about 18.2 fold, about 18.3 fold, about 18.4 fold, about 18.5 fold, about 18.6 fold, about 18.7 fold, about 18.8 fold, about 18.9 fold, about 19.0 fold, about 19.1 fold, about 19.2 fold, about 19.3 fold, about 19.4 fold, about 19.5 fold, about 19.6 fold, about 19.7 fold, about 19.8 fold, about 19.9 fold or about 20.0 fold greater than the binding affinity of an amino acid sequence as set out in SEQ ID NO: 1 to CDH17.

In some embodiments, the anti-CDH17 antibody or antigen-binding fragment thereof has a KD for binding to CDH17 of about 1.0×10⁻⁹ to 5.0×10⁻⁸ M. In some embodiments, the anti-CDH17 antibody or antigen-binding fragment thereof has a KD for binding to CDH17 of about 1.0×10⁻⁹ M, about 2.0×10⁻⁹ M, about 3.0×10⁻⁹ M, about 4.0×10⁻⁹ M, about 5.0×10⁻⁹ M, about 6.0×10⁻⁹ M, about 7.0×10⁻⁹ M, about 8.0×10⁻⁹ M, about 9.0×10⁻⁹ M, about 10.0×10⁻⁹ M, about 11.0×10⁻⁹ M, about 12.0×10⁻⁹ M, about 13.0×10⁻⁹ M, about 14.0×10⁻⁹ M, about 15.0×10⁻⁹ M, about 16.0×10⁻⁹ M, about 17.0×10⁻⁹ M, about 18.0×10⁻⁹ M, about 19.0×10⁻⁹ M, about 20.0×10⁻⁹ M, about 21.0×10⁻⁹ M, about 22.0×10⁻⁹ M, about 23.0×10⁻⁹ M, about 24.0×10⁻⁹ M, about 25.0×10⁻⁹ M, about 26.0×10⁻⁹ M, about 27.0×10⁻⁹ M, about 28.0×10⁻⁹ M, about 29.0×10⁻⁹ M, about 30.0×10⁻⁹ M, about 31.0×10⁻⁹ M, about 32.0×10⁻⁹ M, about 33.0×10⁻⁹ M, about 34.0×10⁻⁹ M, about 35.0×10⁻⁹ M, about 36.0×10⁻⁹ M, about 37.0×10⁻⁹ M, about 38.0×10⁻⁹ M, about 39.0×10⁻⁹ M, about 40.0×10⁻⁹ M, about 41.0×10⁻⁹ M, about 42.0×10⁻⁹ M, about 43.0×10⁻⁹ M, about 44.0×10⁻⁹ M, about 45.0×10⁻⁹ M, about 46.0×10⁻⁹ M, about 47.0×10⁻⁹ M, about 48.0×10⁻⁹ M, about 49.0×10⁻⁹ M, or about 50.0×10⁻⁹ M. In some embodiments, the anti-CDH17 antibody or antigen-binding fragment thereof has a KD for binding to CDH17 of less than 1.0×10⁻⁹ M, about 2.0×10⁻⁹ M, about 3.0×10⁻⁹ M, about 4.0×10⁻⁹ M, about 5.0×10⁻⁹ M, about 6.0×10⁻⁹ M, about 7.0×10⁻⁹ M, about 8.0×10⁻⁹ M, about 9.0×10⁻⁹ M, about 10.0×10⁻⁹ M, about 11.0×10⁻⁹ M, about 12.0×10⁻⁹ M, about 13.0×10⁻⁹ M, about 14.0×10⁻⁹ M, about 15.0×10⁻⁹ M, about 16.0×10⁻⁹ M, about 17.0×10⁻⁹ M, about 18.0×10⁻⁹ M, about 19.0×10⁻⁹ M, about 20.0×10⁻⁹ M, about 21.0×10⁻⁹ M, about 22.0×10⁻⁹ M, about 23.0×10⁻⁹ M, about 24.0×10⁻⁹ M, about 25.0×10⁻⁹ M, about 26.0×10⁻⁹ M, about 27.0×10⁻⁹ M, about 28.0×10⁻⁹ M, about 29.0×10⁻⁹ M, about 30.0×10⁻⁹ M, about 31.0×10⁻⁹ M, about 32.0×10⁻⁹ M, about 33.0×10⁻⁹ M, about 34.0×10⁻⁹ M, about 35.0×10⁻⁹ M, about 36.0×10⁻⁹ M, about 37.0×10⁻⁹ M, about 38.0×10⁻⁹ M, about 39.0×10⁻⁹ M, about 40.0×10⁻⁹ M, about 41.0×10⁻⁹ M, about 42.0×10⁻⁹ M, about 43.0×10⁻⁹ M, about 44.0×10⁻⁹ M, about 45.0×10⁻⁹ M, about 46.0×10⁻⁹ M, about 47.0×10⁻⁹ M, about 48.0×10⁻⁹ M, about 49.0×10⁻⁹ M, or about 50.0×10⁻⁹ M. In some embodiments, the anti-CDH17 antibody or antigen-binding fragment thereof has a KD for binding to CDH17 of at most 1.0×10⁻⁹ M, about 2.0×10⁻⁹ M, about 3.0×10⁻⁹ M, about 4.0×10⁻⁹ M, about 5.0×10⁻⁹ M, about 6.0×10⁻⁹ M, about 7.0×10⁻⁹ M, about 8.0×10⁻⁹ M, about 9.0×10⁻⁹ M, about 10.0×10⁻⁹ M, about 11.0×10⁻⁹ M, about 12.0×10⁻⁹ M, about 13.0×10⁻⁹ M, about 14.0×10⁻⁹ M, about 15.0×10⁻⁹ M, about 16.0×10⁻⁹ M, about 17.0×10⁻⁹ M, about 18.0×10⁻⁹ M, about 19.0×10⁻⁹ M, about 20.0×10⁻⁹ M, about 21.0×10⁻⁹ M, about 22.0×10⁻⁹ M, about 23.0×10⁻⁹ M, about 24.0×10⁻⁹ M, about 25.0×10⁻⁹ M, about 26.0×10⁻⁹ M, about 27.0×10⁻⁹ M, about 28.0×10⁻⁹ M, about 29.0×10⁻⁹ M, about 30.0×10⁻⁹ M, about 31.0×10⁻⁹ M, about 32.0×10⁻⁹ M, about 33.0×10⁻⁹ M, about 34.0×10⁻⁹ M, about 35.0×10⁻⁹ M, about 36.0×10⁻⁹ M, about 37.0×10⁻⁹ M, about 38.0×10⁻⁹ M, about 39.0×10⁻⁹ M, about 40.0×10⁻⁹ M, about 41.0×10⁻⁹ M, about 42.0×10⁻⁹ M, about 43.0×10⁻⁹ M, about 44.0×10⁻⁹ M, about 45.0×10⁻⁹ M, about 46.0×10⁻⁹ M, about 47.0×10⁻⁹ M, about 48.0×10⁻⁹ M, about 49.0×10⁻⁹ M, or about 50.0×10⁻⁹ M.

TABLE 5
KD value for binding CDH17

| Construct Name | SEQ ID NO: | KD, pH 7.4 (M) |
|---|---|---|
| CDH17_vhh_0001 | 1 | 2.72E−08 |
| CDH17_vhh_0951 | 2 | 1.81E−09 |
| CDH17_vhh_0947 | 3 | 1.84E−09 |
| CDH17_vhh_0949 | 4 | 2.01E−09 |
| CDH17_vhh_0946 | 5 | 2.04E−09 |
| CDH17_vhh_0945 | 6 | 2.15E−09 |
| CDH17_vhh_0648 | 7 | 2.18E−09 |
| CDH17_vhh_0650 | 8 | 2.45E−09 |
| CDH17_vhh_0646 | 9 | 2.63E−09 |
| CDH17_vhh_0952 | 10 | 2.61E−09 |
| CDH17_vhh_0677 | 11 | 2.65E−09 |
| CDH17_vhh_0271 | 12 | 2.82E−09 |
| CDH17_vhh_0622 | 13 | 2.93E−09 |
| CDH17_vhh_0653 | 14 | 3.12E−09 |
| CDH17_vhh_0591 | 15 | 3.21E−09 |
| CDH17_vhh_0644 | 16 | 3.32E−09 |
| CDH17_vhh_0652 | 17 | 3.30E−09 |
| CDH17_vhh_0651 | 18 | 3.71E−09 |
| CDH17_vhh_0672 | 19 | 3.76E−09 |
| CDH17_vhh_0674 | 20 | 3.79E−09 |
| CDH17_vhh_0950 | 21 | 3.81E−09 |
| CDH17_vhh_0675 | 22 | 3.89E−09 |
| CDH17_vhh_0263 | 23 | 3.94E−09 |
| CDH17_vhh_0948 | 24 | 3.94E−09 |
| CDH17_vhh_0274 | 25 | 4.35E−09 |
| CDH17_vhh_0676 | 26 | 4.36E−09 |
| CDH17_vhh_0670 | 27 | 4.53E−09 |
| CDH17_vhh_0259 | 28 | 4.75E−09 |
| CDH17_vhh_0372 | 29 | 4.85E−09 |
| CDH17_vhh_0613 | 30 | 4.85E−09 |
| CDH17_vhh_0632 | 31 | 4.90E−09 |
| CDH17_vhh_0689 | 32 | 4.83E−09 |
| CDH17_vhh_0173 | 33 | 4.94E−09 |
| CDH17_vhh_0629 | 34 | 4.96E−09 |
| CDH17_vhh_0267 | 35 | 5.08E−09 |
| CDH17_vhh_0587 | 36 | 5.01E−09 |
| CDH17_vhh_0262 | 37 | 5.14E−09 |
| CDH17_vhh_0645 | 38 | 5.17E−09 |
| CDH17_vhh_0395 | 30 | 5.47E−09 |
| CDH17_vhh_0688 | 40 | 5.43E−09 |
| CDH17_vhh_0156 (BHB156) | 109 | 7.59E−09 |

In some embodiments, the anti-CDH17 antibody or antigen-binding fragment thereof inhibits CDH17 activity with an IC₅₀ of 50.0×10⁻¹² to 1000.0×10⁻¹² M. In some embodiments, the anti-CDH17 antibody or antigen-binding fragment thereof inhibits CDH17 activity with an IC₅₀ of about 50.0×10⁻¹² M, about 60.0×10⁻¹² M, about 70.0×10⁻¹² M, about 80.0×10⁻¹² M, about 90.0×10⁻¹² M, about 100.0×10⁻¹² M, about 150.0×10⁻¹² M, about 200.0×10⁻¹² M, about 250.0×10⁻¹² M, about 300.0×10⁻¹² M, about 350.0×10⁻¹² M, about 400.0×10⁻¹² M, about 450.0×10⁻¹² M, about 500.0×10⁻¹² M, about 550.0×10⁻¹² M, about 600.0×10⁻¹² M, about 650.0×10⁻¹² M, about 700.0×10⁻¹² M, about 750.0×10⁻¹² M, about 800.0×10⁻¹² M, about 850.0×10⁻¹² M, about 900.0×10⁻¹² M, about 950.0×10⁻¹² M, or about 1000.0×10⁻¹² M. In some embodiments, the anti-CDH17 antibody or antigen-binding fragment thereof inhibits CDH17 activity with an IC₅₀ of less than about 50.0×10⁻¹² M, about 60.0×10⁻¹² M, about 70.0×10⁻¹² M, about 80.0×10⁻¹² M, about 90.0×10⁻¹² M, about 100.0×10⁻¹² M, about 150.0×10⁻¹² M, about 200.0×10⁻¹² M, about 250.0×10⁻¹² M, about 300.0×10⁻¹² M, about 350.0×10⁻¹² M, about 400.0×10⁻¹² M, about 450.0×10⁻¹² M, about 500.0×10⁻¹² M, about 550.0×10⁻¹² M, about 600.0×10⁻¹² M, about 650.0×10⁻¹² M, about 700.0×10⁻¹² M, about 750.0×10⁻¹² M, about 800.0×10⁻¹² M, about 850.0×10⁻¹² M, about 900.0×10⁻¹² M, about 950.0×10⁻¹² M, or about 1000.0×10⁻¹² M. In some embodiments, the anti-CDH17 antibody or antigen-binding fragment thereof inhibits CDH17 activity with an IC₅₀ of at most about 50.0×10⁻¹² M, about 60.0×10⁻¹² M, about 70.0×10⁻¹² M, about 80.0×10⁻¹² M, about 90.0×10⁻¹² M, about 100.0×10⁻¹² M, about 150.0×10⁻¹² M, about 200.0×10⁻¹² M, about 250.0×10⁻¹² M, about 300.0×10⁻¹² M, about 350.0×10⁻¹² M, about 400.0×10⁻¹² M, about 450.0×10⁻¹² M, about 500.0×10⁻¹² M, about 550.0×10⁻¹² M, about 600.0×10⁻¹² M, about 650.0×10⁻¹² M, about 700.0×10⁻¹² M, about 750.0×10⁻¹² M, about 800.0×10⁻¹² M, about 850.0×10⁻¹² M, about 900.0×10⁻¹² M, about 950.0×10⁻¹² M, or about 1000.0×10⁻¹² M.

In some embodiments, the IC₅₀ of the anti-CDH17 antibody or antigen-binding fragment thereof to CDH17 is about 5-fold to about 30-fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is about 5 fold to about 10 fold, about 5 fold to about 15 fold, about 5 fold to about 20 fold, about 5 fold to about 25 fold, about 5 fold to about 30 fold, about 10 fold to about 15 fold, about 10 fold to about 20 fold, about 10 fold to about 25 fold, about 10 fold to about 30 fold, about 15 fold to about 20 fold, about 15 fold to about 25 fold, or about 15 fold to about 30 fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, or about 30 fold lesser than the IC₅₀ of an amino acid sequence as set out in SEQ ID NO: 1 to CDH17. In some embodiments, the IC₅₀ of the anti-CDH17 antibody or antigen-binding fragment thereof to CDH17 is at least about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, or about 30 fold lesser than the IC₅₀ of an amino acid sequence as set out in SEQ ID NO: 1 to CDH17.

In some embodiments, the anti-CDH17 antibody or antigen-binding fragment thereof has increased stability compared to stability of an amino acid sequence as set out in SEQ ID NO: 1. In some embodiments, the stability of the anti-CDH17 antibody or antigen-binding fragment thereof to CDH17 is measured by melting temperature (Tm). In some embodiments, the anti-CDH17 antibody or antigen-binding fragment thereof has a Tm between about 40-80° C. In some embodiments, the anti-CDH17 antibody or antigen-binding fragment thereof has a Tm of about 40° C. In some embodiments, the anti-CDH17 antibody or antigen-binding fragment thereof has a Tm of about 45° C. In some embodiments, the anti-CDH17 antibody or antigen-binding fragment thereof has a Tm of about 50° C. In some embodiments, the anti-CDH17 antibody or antigen-binding fragment thereof has a Tm of about 60° C. In some embodiments, the anti-CDH17 antibody or antigen-binding fragment thereof has a Tm of about 65° C. In some embodiments, the anti-CDH17 antibody or antigen-binding fragment thereof has a Tm of about 70° C. In some embodiments, the anti-CDH17 antibody or antigen-binding fragment thereof has a Tm of about 75° C. In some embodiments, the anti-CDH17 antibody or antigen-binding fragment thereof has a Tm of about 80° C. In some embodiments, the anti-CDH17 antibody or antigen-binding fragment thereof has a Tm of less than about 40° C. In some embodiments, the anti-CDH17 antibody or antigen-binding fragment thereof has a Tm of less than about 45° C. In some embodiments, the anti-CDH17 antibody or antigen-binding fragment thereof has a Tm of less than about 50° C. In some embodiments, the anti-CDH17 antibody or antigen-binding fragment thereof has a Tm of less than about 60° C. In some embodiments, the anti-CDH17 antibody or antigen-binding fragment thereof has a Tm of less than about 65° C. In some embodiments, the anti-CDH17 antibody or antigen-binding fragment thereof has a Tm of less than about 70° C. In some embodiments, the anti-CDH17 antibody or antigen-binding fragment thereof has a Tm of less than about 75° C. In some embodiments, the anti-CDH17 antibody or antigen-binding fragment thereof has a Tm of less than about 80° C.

TABLE 6
Melting Temperature

| Construct Name | SEQ ID NO: | Tm (° C.) |
|---|---|---|
| CDH17_vhh_0001 | 1 | 71.3 Â ± 0.151 |
| CDH17_vhh_0951 | 2 | 69.7 |
| CDH17_vhh_0947 | 3 | 63.1 |
| CDH17_vhh_0949 | 4 | 70.6 |
| CDH17_vhh_0946 | 5 | 0 |
| CDH17_vhh_0945 | 6 | 65.3 |
| CDH17_vhh_0648 | 7 | 67.2 |
| CDH17_vhh_0650 | 8 | 64.6 |
| CDH17_vhh_0646 | 9 | n.d. |
| CDH17_vhh_0952 | 10 | 69.7 |
| CDH17_vhh_0677 | 11 | 68 |
| CDH17_vhh_0271 | 12 | 67.5 |
| CDH17_vhh_0622 | 13 | n.d. |
| CDH17_vhh_0653 | 14 | n.d. |
| CDH17_vhh_0591 | 15 | 71.6 |
| CDH17_vhh_0644 | 16 | n.d. |
| CDH17_vhh_0652 | 17 | n.d. |
| CDH17_vhh_0651 | 18 | 59.3 |
| CDH17_vhh_0672 | 19 | 68.7 |
| CDH17_vhh_0674 | 20 | 66.7 |
| CDH17_vhh_0950 | 21 | 65 |
| CDH17_vhh_0675 | 22 | 70.7 |
| CDH17_vhh_0263 | 23 | 70.4 Â ± 0.255 |
| CDH17_vhh_0948 | 24 | 67.7 |
| CDH17_vhh_0274 | 25 | 64.3 Â ± 4.538 |
| CDH17_vhh_0676 | 26 | 67.1 |
| CDH17_vhh_0670 | 27 | 66.5 |
| CDH17_vhh_0259 | 28 | 66.9 |
| CDH17_vhh_0372 | 29 | 66.3 |
| CDH17_vhh_0613 | 30 | 67.4 |
| CDH17_vhh_0632 | 31 | n.d. |
| CDH17_vhh_0689 | 32 | n.d. |
| CDH17_vhh_0173 | 33 | 62.3 Â ± 0.745 |
| CDH17_vhh_0629 | 34 | n.d. |
| CDH17_vhh_0267 | 35 | 47.3 |
| CDH17_vhh_0587 | 36 | 65.1 |
| CDH17_vhh_0262 | 37 | 67.7 Â ± 0.280 |
| CDH17_vhh_0645 | 38 | n.d. |
| CDH17_vhh_0395 | 30 | 69 |
| CDH17_vhh_0688 | 40 | 66.4 |
| CDH17_vhh_0156 (BHB156) | 109 | 68.9 Â ± 0.391 |

(n.d. means not determined)

In some embodiments, the anti-CDH17 antibody or antigen-binding fragment thereof has a higher purity compared to a purity of an amino acid sequence as set out in SEQ ID NO: 1. In some embodiments, purity is measured via capillary gel electrophoresis using sodium dodecyl sulfate (CE-SDS). In some embodiments, the anti-CDH17 antibody or antigen-binding fragment thereof has a purity of 70-100%. In some embodiments, the anti-CDH17 antibody or antigen-binding fragment thereof has a purity of 70%. In some embodiments, the anti-CDH17 antibody or antigen-binding fragment thereof has a purity of 71%. In some embodiments, the anti-CDH17 antibody or antigen-binding fragment thereof has a purity of 72%. In some embodiments, the anti-CDH17 antibody or antigen-binding fragment thereof has a purity of 73%. In some embodiments, the anti-CDH17 antibody or antigen-binding fragment thereof has a purity of 74%. In some embodiments, the anti-CDH17 antibody or antigen-binding fragment thereof has a purity of 75%. In some embodiments, the anti-CDH17 antibody or antigen-binding fragment thereof has a purity of 76%. In some embodiments, the anti-CDH17 antibody or antigen-binding fragment thereof has a purity of 77%. In some embodiments, the anti-CDH17 antibody or antigen-binding fragment thereof has a purity of 78%. In some embodiments, the anti-CDH17 antibody or antigen-binding fragment thereof has a purity of 79%. In some embodiments, the anti-CDH17 antibody or antigen-binding fragment thereof has a purity of 80%. In some embodiments, the anti-CDH17 antibody or antigen-binding fragment thereof has a purity of 81%. In some embodiments, the anti-CDH17 antibody or antigen-binding fragment thereof has a purity of 82%. In some embodiments, the anti-CDH17 antibody or antigen-binding fragment thereof has a purity of 83%. In some embodiments, the anti-CDH17 antibody or antigen-binding fragment thereof has a purity of 84%. In some embodiments, the anti-CDH17 antibody or antigen-binding fragment thereof has a purity of 85%. In some embodiments, the anti-CDH17 antibody or antigen-binding fragment thereof has a purity of 86%. In some embodiments, the anti-CDH17 antibody or antigen-binding fragment thereof has a purity of 87%. In some embodiments, the anti-CDH17 antibody or antigen-binding fragment thereof has a purity of 88%. In some embodiments, the anti-CDH17 antibody or antigen-binding fragment thereof has a purity of 89%. In some embodiments, the anti-CDH17 antibody or antigen-binding fragment thereof has a purity of 90%. In some embodiments, the anti-CDH17 antibody or antigen-binding fragment thereof has a purity of 91%. In some embodiments, the anti-CDH17 antibody or antigen-binding fragment thereof has a purity of 92%. In some embodiments, the anti-CDH17 antibody or antigen-binding fragment thereof has a purity of 93%. In some embodiments, the anti-CDH17 antibody or antigen-binding fragment thereof has a purity of 94%. In some embodiments, the anti-CDH17 antibody or antigen-binding fragment thereof has a purity of 95%. In some embodiments, the anti-CDH17 antibody or antigen-binding fragment thereof has a purity of 96%. In some embodiments, the anti-CDH17 antibody or antigen-binding fragment thereof has a purity of 97%. In some embodiments, the anti-CDH17 antibody or antigen-binding fragment thereof has a purity of 98%. In some embodiments, the anti-CDH17 antibody or antigen-binding fragment thereof has a purity of 99%. In some embodiments, the anti-CDH17 antibody or antigen-binding fragment thereof has a purity of 100%.

In some embodiments, the anti-CDH17 antibody or antigen-binding fragment thereof has a purity of more than 70%. In some embodiments, the anti-CDH17 antibody or antigen-binding fragment thereof has a purity of more than 71%. In some embodiments, the anti-CDH17 antibody or antigen-binding fragment thereof has a purity of more than 72%. In some embodiments, the anti-CDH17 antibody or antigen-binding fragment thereof has a purity of more than 73%. In some embodiments, the anti-CDH17 antibody or antigen-binding fragment thereof has a purity of more than 74%. In some embodiments, the anti-CDH17 antibody or antigen-binding fragment thereof has a purity of more than 75%. In some embodiments, the anti-CDH17 antibody or antigen-binding fragment thereof has a purity of more than 76%. In some embodiments, the anti-CDH17 antibody or antigen-binding fragment thereof has a purity of more than 77%. In some embodiments, the anti-CDH17 antibody or antigen-binding fragment thereof has a purity of more than 78%. In some embodiments, the anti-CDH17 antibody or antigen-binding fragment thereof has a purity of more than 79%. In some embodiments, the anti-CDH17 antibody or antigen-binding fragment thereof has a purity of more than 80%. In some embodiments, the anti-CDH17 antibody or antigen-binding fragment thereof has a purity of more than 81%. In some embodiments, the anti-CDH17 antibody or antigen-binding fragment thereof has a purity of more than 82%. In some embodiments, the anti-CDH17 antibody or antigen-binding fragment thereof has a purity of more than 83%. In some embodiments, the anti-CDH17 antibody or antigen-binding fragment thereof has a purity of more than 84%. In some embodiments, the anti-CDH17 antibody or antigen-binding fragment thereof has a purity of more than 85%. In some embodiments, the anti-CDH17 antibody or antigen-binding fragment thereof has a purity of more than 86%. In some embodiments, the anti-CDH17 antibody or antigen-binding fragment thereof has a purity of more than 87%. In some embodiments, the anti-CDH17 antibody or antigen-binding fragment thereof has a purity of more than 88%. In some embodiments, the anti-CDH17 antibody or antigen-binding fragment thereof has a purity of more than 89%. In some embodiments, the anti-CDH17 antibody or antigen-binding fragment thereof has a purity of more than 90%. In some embodiments, the anti-CDH17 antibody or antigen-binding fragment thereof has a purity of more than 91%. In some embodiments, the anti-CDH17 antibody or antigen-binding fragment thereof has a purity of more than 92%. In some embodiments, the anti-CDH17 antibody or antigen-binding fragment thereof has a purity of more than 93%. In some embodiments, the anti-CDH17 antibody or antigen-binding fragment thereof has a purity of more than 94%. In some embodiments, the anti-CDH17 antibody or antigen-binding fragment thereof has a purity of more than 95%. In some embodiments, the anti-CDH17 antibody or antigen-binding fragment thereof has a purity of more than 96%. In some embodiments, the anti-CDH17 antibody or antigen-binding fragment thereof has a purity of more than 97%. In some embodiments, the anti-CDH17 antibody or antigen-binding fragment thereof has a purity of more than 98%. In some embodiments, the anti-CDH17 antibody or antigen-binding fragment thereof has a purity of more than 99%.

TABLE 7
Purity measured via capillary gel electrophoresis
using sodium dodecyl sulfate (CE-SDS)

| Construct Name | SEQ ID NO: | Purity (%) |
|---|---|---|
| CDH17_vhh_0001 | 1 | 95.3 |
| CDH17_vhh_0951 | 2 | 100 |
| CDH17_vhh_0947 | 3 | 100 |
| CDH17_vhh_0949 | 4 | 100 |
| CDH17_vhh_0946 | 5 | 100 |
| CDH17_vhh_0945 | 6 | 100 |
| CDH17_vhh_0648 | 7 | 100 |
| CDH17_vhh_0650 | 8 | 77.6 |
| CDH17_vhh_0646 | 9 | 100 |
| CDH17_vhh_0952 | 10 | 100 |
| CDH17_vhh_0677 | 11 | 100 |
| CDH17_vhh_0271 | 12 | 97.7 |
| CDH17_vhh_0622 | 13 | 99 |
| CDH17_vhh_0653 | 14 | 100 |
| CDH17_vhh_0591 | 15 | 100 |
| CDH17_vhh_0644 | 16 | 100 |
| CDH17_vhh_0652 | 17 | 100 |
| CDH17_vhh_0651 | 18 | 100 |
| CDH17_vhh_0672 | 19 | 100 |
| CDH17_vhh_0674 | 20 | 100 |
| CDH17_vhh_0950 | 21 | 100 |
| CDH17_vhh_0675 | 22 | 100 |
| CDH17_vhh_0263 | 23 | 99.3 |
| CDH17_vhh_0948 | 24 | 100 |
| CDH17_vhh_0274 | 25 | 100 |
| CDH17_vhh_0676 | 26 | 100 |
| CDH17_vhh_0670 | 27 | 100 |
| CDH17_vhh_0259 | 28 | 86.1 |
| CDH17_vhh_0372 | 29 | 98.5 |
| CDH17_vhh_0613 | 30 | 100 |
| CDH17_vhh_0632 | 31 | 100 |
| CDH17_vhh_0689 | 32 | 100 |
| CDH17_vhh_0173 | 33 | 98.4 |
| CDH17_vhh_0629 | 34 | 100 |
| CDH17_vhh_0267 | 35 | 96.3 |
| CDH17_vhh_0587 | 36 | 97.1 |
| CDH17_vhh_0262 | 37 | 97.7 |
| CDH17_vhh_0645 | 38 | 98 |
| CDH17_vhh_0395 | 30 | 89.8 |
| CDH17_vhh_0688 | 40 | 100 |
| CDH17_vhh_0156 | 109 | 100 |

### Bispecific T Cell Engager & Antibody Drug Conjugate

In recent years, methods of diagnosing or treating various diseases using antibodies have been studied. Particularly, because of the target specificity of antibodies, various types of drugs containing antibodies have been developed. Amongst the various antibody formats developed, bispecific T-cell engagers (BiTEs) and antibody drug conjugates (ADCs) show much promise. BiTEs are bi-specific molecules that are specific for the CD38 subunit of the TCR complex of a T-cell and also a target an antigen of interest, such as a cancer antigen. Since BiTEs are specific for the TCR complex, this enables BiTEs to activate resident T-cells to kill cells expressing a particular target antigen on their cell surface, for example cancer cells. Antibody drug conjugates (ADCs) are complex molecules composed of an antibody linked to a biologically active payload or drug (e.g., a cytotoxic agent). Unlike the payload or drug alone, in some embodiments the antigen specificity of the antibody portion of the ADC enables the discrimination between healthy and diseased tissue reducing off-target effects including toxicity.

The anti-CDH17 antibody or antigen-binding fragment thereof described herein may be a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (scFv), an intrabody, an anti-idiotypic (anti-Id) antibody, a VHH antibody, or ab antigen-binding fragment thereof.

A single chain Fv molecule (scFv) can comprise a VL domain and a VH domain, which may be associated to form a target binding site. These two domains may be further linked by a linker (L). In some embodiments, the anti-CDH17 antibody or fragment thereof may comprise a linker peptide. In some instances, the linker may be a flexible linker, a rigid linker, a non-cleavable linker or a cleavable linker. In some embodiments, the linker peptide may link a portion of a heavy chain variable domain to a portion of a light chain variable domain. In some embodiments, a heterodimeric Fc may comprise a first Fc polypeptide linked to the first antigen-binding polypeptide construct (e.g., CD3) with or without a first linker and a second Fc polypeptide linked to the second antigen-binding polypeptide construct (e.g., CDH17) with or without a second linker. The antibody described herein can be optimized for protein expression and yield by changing composition and/or length of the polypeptide linker. In some embodiments, the polypeptide linker can be about 2 amino acids to about 24 amino acids. In some embodiments, the polypeptide linker may be about 2 amino acids to about 4 amino acids, about 2 amino acids to about 6 amino acids, about 2 amino acids to about 8 amino acids, about 2 amino acids to about 10 amino acids, about 2 amino acids to about 12 amino acids, about 2 amino acids to about 14 amino acids, about 2 amino acids to about 16 amino acids, about 2 amino acids to about 18 amino acids, about 2 amino acids to about 20 amino acids, about 2 amino acids to about 22 amino acids, about 2 amino acids to about 24 amino acids, about 4 amino acids to about 6 amino acids, about 4 amino acids to about 8 amino acids, about 4 amino acids to about 10 amino acids, about 4 amino acids to about 12 amino acids, about 4 amino acids to about 14 amino acids, about 4 amino acids to about 16 amino acids, about 4 amino acids to about 18 amino acids, about 4 amino acids to about 20 amino acids, about 4 amino acids to about 22 amino acids, about 4 amino acids to about 24 amino acids, about 6 amino acids to about 8 amino acids, about 6 amino acids to about 10 amino acids, about 6 amino acids to about 12 amino acids, about 6 amino acids to about 14 amino acids, about 6 amino acids to about 16 amino acids, about 6 amino acids to about 18 amino acids, about 6 amino acids to about 20 amino acids, about 6 amino acids to about 22 amino acids, about 6 amino acids to about 24 amino acids, about 8 amino acids to about 10 amino acids, about 8 amino acids to about 12 amino acids, about 8 amino acids to about 14 amino acids, about 8 amino acids to about 16 amino acids, about 8 amino acids to about 18 amino acids, about 8 amino acids to about 20 amino acids, about 8 amino acids to about 22 amino acids, about 8 amino acids to about 24 amino acids, about 10 amino acids to about 12 amino acids, about 10 amino acids to about 14 amino acids, about 10 amino acids to about 16 amino acids, about 10 amino acids to about 18 amino acids, about 10 amino acids to about 20 amino acids, about 10 amino acids to about 22 amino acids, about 10 amino acids to about 24 amino acids, about 12 amino acids to about 14 amino acids, about 12 amino acids to about 16 amino acids, about 12 amino acids to about 18 amino acids, about 12 amino acids to about 20 amino acids, about 12 amino acids to about 22 amino acids, about 12 amino acids to about 24 amino acids, about 14 amino acids to about 16 amino acids, about 14 amino acids to about 18 amino acids, about 14 amino acids to about 20 amino acids, about 14 amino acids to about 22 amino acids, about 14 amino acids to about 24 amino acids, about 16 amino acids to about 18 amino acids, about 16 amino acids to about 20 amino acids, about 16 amino acids to about 22 amino acids, about 16 amino acids to about 24 amino acids, about 18 amino acids to about 20 amino acids, about 18 amino acids to about 22 amino acids, about 18 amino acids to about 24 amino acids, about 20 amino acids to about 22 amino acids, about 20 amino acids to about 24 amino acids, or about 22 amino acids to about 24 amino acids. In some embodiments, the polypeptide linker can be about 2 amino acids, about 4 amino acids, about 6 amino acids, about 8 amino acids, about 10 amino acids, about 12 amino acids, about 14 amino acids, about 16 amino acids, about 18 amino acids, about 20 amino acids, about 22 amino acids, or about 24 amino acids. In some embodiments, the polypeptide linker can be at least about 2 amino acids, about 4 amino acids, about 6 amino acids, about 8 amino acids, about 10 amino acids, about 12 amino acids, about 14 amino acids, about 16 amino acids, about 18 amino acids, about 20 amino acids, or about 22 amino acids. In some embodiments, the polypeptide linker may at most about 4 amino acids, about 6 amino acids, about 8 amino acids, about 10 amino acids, about 12 amino acids, about 14 amino acids, about 16 amino acids, about 18 amino acids, about 20 amino acids, about 22 amino acids, or about 24 amino acids. In some embodiments, the linker peptide may comprise (G₄S)ₙ, (SG₄)ₙ, G₄(SG₄)ₙ or G₂(SG₂)ₙ, wherein n can be 1, 2, 3, 4, 5, 6, 7, 8, 9, or 10. In some embodiments, the linker peptide comprises GGGGSGGGGS (SEQ ID NO: 100). In some embodiments, the linker peptide comprises GGGGS (SEQ ID NO: 101). Exemplary linker peptide sequences are listed in Table 8 below.

TABLE 8

| | SEQ ID |
|---|---|
| Sequence | NO: |
| GGGGSGGGGS | 100 |
| GGGGS | 101 |

In some embodiments, the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof.

In some embodiments, the antigen-binding domain can be humanized or human. In some embodiments, the antigen-binding domain may not be humanized or not human.

The anti-CDH17 antibodies or antigen-fragment thereof can be of various isotypes, preferably human IgG1, IgG2, IgG3 or IgG4, more preferably comprising human IgG1 hinge and constant region sequences. The antibodies or fragments thereof can be chimeric human-mouse, humanized (human framework and murine hypervariable (CDR) regions), or fully human, as well as variations thereof, such as half-IgG4 antibodies (referred to as “unibodies). More preferably, the antibodies or fragments thereof may be designed or selected to comprise human constant region sequences that belong to specific allotypes, which may result in reduced immunogenicity when administered to a human subject. Preferred allotypes for administration include a non-Gim1 allotype (nGlm1), such as Glm3, Glm3,1, G1m3,2 or G1m3,1,2. More preferably, the allotype is selected from the group consisting of the nG1m1, G1m3, nGlm1,2 and Km3 allotypes.

In some embodiments, the T-cell redirecting bispecific antibodies comprising the anti-CDH17 and or antigen-binding fragment thereof as described herein may be administered to a subject for diagnosis or treatment of a condition. Exemplary conditions may include, but not limited to, cancer, hyperplasia, neurodegenerative disease, Alzheimer's disease, cardiovascular disease, metabolic disease, vasculitis, viral infection, fungal infection, bacterial infection, diabetic retinopathy, macular degeneration, autoimmune disease, edema, pulmonary hypertension, sepsis, myocardial angiogenesis, plaque neovascularization, restenosis, neointima formation after vascular trauma, telangiectasia, hemophiliac joints, angiofibroma, fibrosis associated with chronic inflammation, lung fibrosis, deep venous thrombosis or wound granulation.

In some embodiments, the anti-CDH17 antibody or antigen-binding fragment thereof described herein may be of use for diagnostic or therapy of cancer. Exemplary types of cancers that may be targeted include pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer, acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer.

In some embodiments, the cancer may comprise a solid tumor, such as adenocarcinoma, colon adenocarcinoma, rectal adenocarcinoma, pancreatic adenocarcinoma, glioblastoma, neuroendocrine tumors, colorectal carcinoma, gastric carcinoma, esophageal carcinoma, neuroblastoma, or osteosarcoma.

In some embodiments, the BsAbs comprising the anti-CDH17 antibody or antigen-binding fragment thereof as described herein may comprise an additional portion that binds to a tumor-associated antigens that may be targeted. The tumor-associated antigen can be, but are not limited to, alpha-fetoprotein (AFP), a-actinin-4, A3, antigen specific for A33 antibody, ART-4, B7, Ba 733, BAGE, BrE3-antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD17, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, CDH1, CDH6, CDH17, CDH19, CDH179b, HIF-1α, colon-specific antigen-p (CSAp), CEA (CEACAM5), CEACAM6, c-Met, DAM, EGFR, EGFRVIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker and an oncogene product.

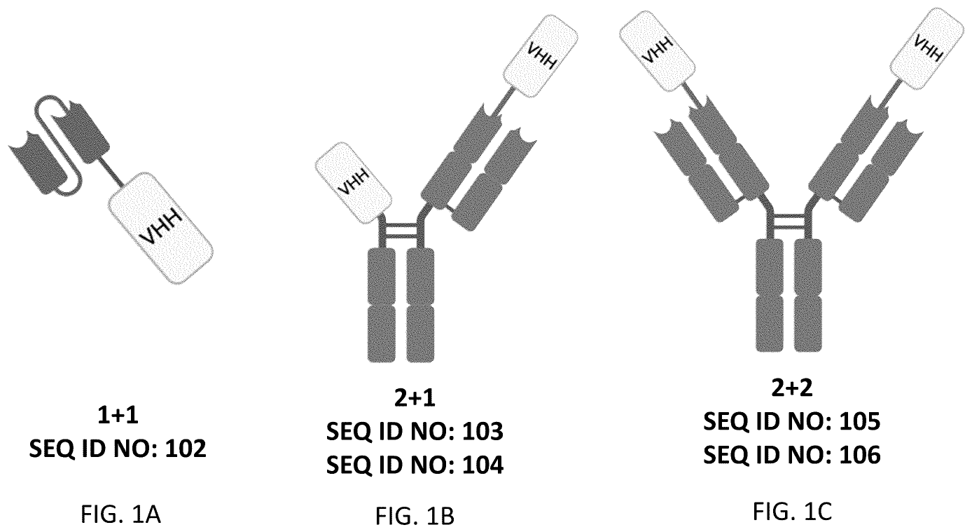

For example, FIG. 1 shows multi specific T cell engager designs demonstrating the possible arrangement of an anti-CDH17 VHH and an anti-CD3 agonist scFv or Fab. FIG. 1 further illustrates a CDH17 x CD3 bispecific T cell engager construct lacking an Fc domain (1+1 geometry) of SEQ ID NO: 102. FIG. 1 further illustrates an asymmetric CDH17 x CD3 bispecific T cell engager construct with an Fc domain (2+1) geometry of SEQ ID NO: 103 and SEQ ID NO: 104. FIG. 1 further illustrates a symmetric CDH17 x CD3 bispecific T cell engager construct with an Fc domain (2+2 geometry) of SEQ ID NO: 105 and SEQ ID NO: 106.

### Antibody Drug Conjugate (ADC)

In some embodiments, an anti-CDH17 antibody or antibody composition is a protein comprising the anti-CDH17 antibody or antigen-binding fragment thereof disclosed herein conjugated to a cytotoxic agent, and/or a Fc domain. In some embodiments, the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB).

In some embodiments, an anti-CDH17 antibody or antibody composition is a protein comprising the anti-CDH17 antibody or antigen-binding fragment thereof conjugated to a cytotoxic agent, and/or a Fc domain by an attachment group. In some embodiments the attachment group is a lysine, cysteine, or glycoconjugate. In some embodiments, the attachment group is on the heavy chain, the light chain, and or the hinge region of the antibody or antigen-binding fragment.

In some embodiments, an anti-CDH17 antibody or antibody composition is a protein comprising the anti-CDH17 antibody or antigen-binding fragment thereof conjugated to a cytotoxic agent, and/or a Fc domain by a linker. In some embodiments, the linker is cleavable or noncleavable. In some embodiments the cleavable linker is a chemically cleavable or enzymatically cleavable linker. In some embodiments, the chemically cleavable linker is pH sensitive or reduction sensitive. In some embodiments the enzymatically cleavable linker is a peptide based linker, a β-glucuronide based linker, or a phosphate based linker. In some embodiments, the cleavable linker is a cleavable disulfide. In some embodiments, β-glucuronide based linker is a β-glucuronidase sensitive linker or a β-galactosidase sensitive linker. In some embodiments the noncleavable linker is a thioether linker or a maleimidocaproyl (mc) linker. In some embodiments, the linker comprises hydrazone, (4-(4′-acetylphenoxy)butanoic acid) hydrazone, a (6-maleimidocaproyl) hydrazone, SPDB, N-E-maleimidocaproic acid hydrazide (EMCH), succinimidyl 3-(2-pyridyldithio)propionate or 3-(2-Pyridyldithio) propionic acid N-hydroxysuccinimide ester (SPDP), 1-[Bis(dimethylamino)methylene]-1H-1,2,3-triazolo[4,5-b]pyridinium 3-oxid hexafluorophosphate (HATU), valine-citrulline (Val-Cit), phenylalanine-lysine (Phe-Lys), valine-alanine, Val-Cit-para-aminobenzyl carbamate (PABC), N-succinimidyl-4-(maleimidomethyl)cyclohexane-1-carboxylate (SMCC), CL2, maleimidohexanoic, maleimidohexanoic-Val-Cit-PABC, 2-aminobenzamidoxime, mercaptoethylpyrazolone, glutamic acid-valine-citrulline (EVCit), or EVCit-PABC.

In some embodiments, an anti-CDH17 antibody or antibody composition is a protein comprising the anti-CDH17 antibody or antigen-binding fragment thereof conjugated to a cytotoxic agent and/or a Fc domain by a linker comprises a spacer. In some embodiments, the spacer comprises para-aminobenzyl carbamate (PABC), cyclodextrin, polymers, PEG or PEG8. The term “ADC composition”, as used herein, refers to a composition containing a heterogeneous mixture of antibody species containing various drug loads (“DL”). The average drug antibody ratio of the entire heterogenous ADC composition is referred to herein as “average DAR” or “DAR”. For example, an ADC composition may comprise of mixture of antibody species each with their own DL (some species in the mixture are DL2, some species in the mixture are DL4, some species in the mixture are DL6, and some species in the mixture are DL8) and the average DAR for the entire composition may be about 4. In some embodiments, the term “percent DL” may be used to describe the percent of a specific DL species within the heterogenous ADC composition (e.g. percent DL2 is 5 about 10% to about 30% of the total heterogenous ADC composition). In some embodiments, drugs or a cytotoxic agent may be conjugated to antibodies or an antigen-binding fragment thereof via sulfhydryl groups. The sulfhydryl groups can be sulfhydryl groups on cysteine side chains. The cysteine residues can be naturally present in an antibody (e.g., interchain disulfides) or introduced by other means, e.g., mutagenesis.

For example, DLO has no drug load on the antibody. For example, DL2 has a drug load of two. For example, DAR4 has a drug antibody ratio of 4. In some embodiments, an antibody or antigen-binding fragment thereof disclosed herein has a drug antibody ratio of 2. In some embodiments, an antibody or antigen-binding fragment thereof disclosed herein has a drug antibody ratio of 4. In some embodiments, an antibody or antigen-binding fragment thereof disclosed herein has a drug antibody ratio of about 1-10. In some embodiments, the antibody or antigen-binding fragment thereof has a drug antibody ratio of about 1. In some embodiments, the antibody or antigen-binding fragment thereof has a drug antibody ratio of about 2. In some embodiments, the antibody or antigen-binding fragment thereof has a drug antibody ratio of about 3. In some embodiments, the antibody or antigen-binding fragment thereof has a drug antibody ratio of about 4. In some embodiments, the antibody or antigen-binding fragment thereof has a drug antibody ratio of about 5. In some embodiments, the antibody or antigen-binding fragment thereof has a drug antibody ratio of about 6. In some embodiments, the antibody or antigen-binding fragment thereof has a drug antibody ratio of about 7. In some embodiments, the antibody or antigen-binding fragment thereof has a drug antibody ratio of about 8. In some embodiments, the antibody or antigen-binding fragment thereof has a drug antibody ratio of about 9. In some embodiments, the antibody or antigen-binding fragment thereof has a drug antibody ratio of about 10. Various methods exist, and are known to those skilled in the art, for calculating the percent DL species and/or average DAR in an ADC composition. For example, hydrophobic interaction chromatography (HIC) which separates DL species based on the number of drugs loaded. LC-MS assays have also been developed to assess DL distribution. Exemplary methods for calculating the drug load distribution in an ADC composition can be found, for example, in Journal of Chromatography B 1060 (2017) 182-189. For example, DL2 has a drug load of two. In one embodiment, the conjugation sites for DL2 comprise HC 224. For example, DL4 has a drug load of four. In one embodiment, the conjugation sites for DL4a comprise HC 224. In one embodiment, the conjugation sites for DL4b comprise HC 233. For example, DL6 has a drug load of six. In one embodiment, the conjugation sites for DL6 comprise HC 224 and HC 233. For example, DL8 has a drug load of eight. In one embodiment, the conjugation sites for DL8 comprise HC 224 and HC 233.

### Method of Making Polypeptides

Also provided herein are methods of producing an polypeptide that include: (a) culturing a cell (e.g., any of the cells described herein) including any of the nucleic acids encoding any of the polypeptides described herein, or any of the expression vectors described herein that include nucleic acid encoding any of the polypeptides described herein, in a culture medium under conditions sufficient to allow for the production of the polypeptide; and (b) harvesting the polypeptide from the host cell or the culture medium. In some embodiments of any of the methods described herein, the method further includes isolating the polypeptide (e.g., through performance of one or more column chromatography steps, ultrafiltration/diafiltration, and/or viral inactivation). In some embodiments of any of the methods described herein, the method further includes formulating the isolated polypeptide into a composition (e.g., a pharmaceutical composition).

Any of the polypeptides described herein can be produced by any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell. For example, a host cell can be a CHO cell or a HEK cell.

Methods of culturing cells are well known in the art. Cells can be maintained in vitro under conditions that favor cell proliferation, cell growth, and/or cell differentiation. For example, cells can be cultured by contacting a cell (e.g., any of the cells described herein) with a cell culture medium that includes supplemental growth factors to support cell viability and cell growth.

Methods of introducing nucleic acids (e.g., any of the exemplary nucleic acids described herein) and/or expression vectors (e.g., any of the exemplary expression vectors described herein (e.g., an AAV vector)) into cells (e.g., mammalian cells) are known in the art. Non-limiting examples of methods that can be used to introduce a nucleic acid (e.g., DNA, RNA, ssRNA, siRNA, microRNA, or mRNA) and/or an expression vector (e.g., any of the exemplary expression vectors described herein (e.g., an AAV vector) include: electroporation, lipofection, transfection, microinjection, calcium phosphate transfection, dendrimer-based transfection, anionic polymer transfection, cationic polymer transfection, transfection using highly branched organic compounds, cell-squeezing, sonoporation, optical transfection, magnetofection, particle-based transfection (e.g., nanoparticle transfection), transfection using liposomes (e.g., cationic liposomes), and viral transduction (e.g., lentiviral transduction, adenoviral transduction).

Some methods described herein further include isolating or purifying the polypeptide from cell culture medium or from a cell (e.g., a mammalian cell) using techniques well-known in the art (e.g., ion exchange chromatography (anionic or cation), metal-affinity chromatography, ligand-affinity chromatography, size exclusion chromatography, hydrophobic interaction chromatography, and precipitation (e.g., ammonium sulfate precipitation, polyethylene glycol precipitation).

### Method of Treatment

Described herein is the pharmaceutical composition for use in a therapy includes an antibody monotherapy, antibody-drug conjugate (ADC) therapy, T cell-engaging immunotherapy, or CAR-T therapy. In some embodiments, the pharmaceutical compositions may further comprise at least one additional therapeutic agent.

In some embodiments, compositions comprising antibodies can be provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, compositions comprising antibodies may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1. In some embodiments, the composition described herein may be manufactured as a medicament for treatment of cancer.

### Pharmaceutical Composition

Also provided herein are compositions (e.g., pharmaceutical compositions) that include any of the polypeptides, vectors, or nucleic acids described herein. Any of the pharmaceutical compositions can include any of the polypeptides, vectors, or nucleic acids described herein and one or more (e.g., 1, 2, 3, 4, or 5) pharmaceutically or physiologically acceptable carriers, diluents, or excipients. In some embodiments, any of the pharmaceutical compositions described herein can include one or more buffers (e.g., a neutral-buffered saline, a phosphate-buffered saline (PBS)), one or more carbohydrates (e.g., glucose, mannose, sucrose, dextran, or mannitol), one or more proteins, polypeptides, or amino acids (e.g., glycine), one or more antioxidants, one or more chelating agents (e.g., glutathione or EDTA), one or more preservatives, and/or a pharmaceutically acceptable carrier (e.g., PBS, saline, or bacteriostatic water).

In some embodiments, any of the pharmaceutical compositions described herein can further include one or more (e.g., 1, 2, 3, 4, or 5) agents that promote the entry of any of the vectors or nucleic acids described herein into a cell (e.g., a mammalian cell) (e.g., a liposome or cationic lipid).

In some embodiments, any of the vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers and poly(lactic-co-glycolic acid) (PLGA) polymers.

The pharmaceutical compositions provided herein can be, e.g., formulated to be compatible with their intended route of administration. In some embodiments, the compositions are formulated for subcutaneous, intramuscular, or intravenous administration. In some examples, the compositions include a therapeutically effective amount of any of the polypeptides, vectors, or nucleic acids described herein. Single or multiple administrations of any of the pharmaceutical compositions described herein can be given (e.g., administered) to a subject depending on, for example, the frequency and the dosage required and tolerated by the subject. A dosage of the pharmaceutical composition including any of the polypeptides described herein, any of the vectors described herein, or any of the nucleic acids described herein should provide a sufficient quantity to effectively ameliorate or treat symptoms, conditions or diseases.

### Kits

A kit-of-parts comprising a pharmaceutical composition together with instructions for use is further provided. For convenience, the kit-of-parts may comprise reagents in predetermined amounts with instructions for use.

In some embodiments, disclosed herein are kids comprising an anti-CDH17 antibody or antigen-binding fragment thereof disclosed herein. In some embodiments, a kit can be a diagnostic kit. In some embodiments, a kit comprises an anti-CDH17 antibody or antigen-binding fragment thereof disclosed herein and instructions for use. In some embodiments, a kit comprises means for measuring an anti-CDH17 antibody or antigen-binding fragment thereof level in a sample and instructions for use. A kit may provide a unit or device for obtaining a sample from a subject (e.g., a device with a needle coupled to an aspirator).

A kit may include a plurality of syringes, ampules, foil packets, or blister packs, each containing a single unit dose of a kit component described herein. Containers of a kit may be airtight, waterproof (e.g., impermeable to changes in moistures or evaporation), and/or light-tight. A kit may include a device suitable for administration of the components, e.g., a syringe, inhalant, pipette, forceps, measured spoon, dropper (e.g., eye dropper), swab (e.g., a cotton swab or wooden swab), or any such delivery device. In some embodiments, the device may be a medical implant device, e.g., packaged for surgical insertion. A kit disclosed herein may comprise one or more reagents or instruments which enable the method to be carried out. In some embodiments, reagents or instruments include one or more of the following: suitable buffer(s) (aqueous solutions), a support comprising wells on which quantitative reactions can be done. A kit may be a specific kit for a specific tissue sample. Further, a kit disclosed herein may comprise a control. In some embodiments, the kid may comprise any of the compositions (e.g., pharmaceutical compositions) described herein which include any of the nucleic acids, any of the polypeptides, or any of the vectors described herein. In some embodiments, a kit can include a solid composition (e.g., a lyophilized composition including any of the vectors, polypeptides, or nucleic acids described herein) and a liquid for solubilizing the lyophilized composition. In some embodiments, the kit includes a vial including any of the pharmaceutical compositions described herein (e.g., formulated as an aqueous pharmaceutical composition).

In addition to the above components, instructions for use may be provided in a kit. These instructions may be presented in the kit in a variety of forms, such as printed information on a suitable medium or substate (e.g., a piece or pieces of paper on which the information is printed), in the packaging of the kit, in a package insert, etc. In some embodiments, instructions for use can be provided on a computer readable medium (e.g., jump/thumb drive, CD, etc.), or which the information has been recorded, or at a website address which may be used via the internet to access the information at a website.

### Device

Another aspect of the disclosure provides a pre-filled syringe or autoinjector device, comprising an anti-CDH17 antibody or antigen-binding fragment thereof or a composition described herein. In some embodiments, a composition stored in a container, pre-filled syringe, injector or autoinjector device contains an anti-CDH17 antibody or antigen-binding fragment thereof disclosed herein.

### Arrays

Disclosed herein are supports comprising an anti-CDH17 antibody or antigen-binding fragment thereof disclosed herein. A support can be a solid support. A support may take a variety of configurations ranging from simple to complex, depending on the intended use of the support. A support can have an overall slide or plate configuration, such as a rectangular or disc configuration. A standard microplate configuration can be used. In some embodiments, the surface may be smooth or substantially planar, or have irregularities, such as depressions or elevations. In some embodiments, a support may have a rectangular cross-sectional shape, having a length of from about 10-200 mm, 40-150 mm, or 75-125 mm; a width of from about 10-200 mm, 20-120 mm, or 25-80 mm, and a thickness of from about 0.01-5.0 mm, 0.1-2 mm, or 0.2 to 1 mm.

In some embodiments, a support can be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A support as described herein can be formed of any suitable material, including metals, metal oxides, semiconductors, polymers (particularly organic polymers in any suitable form including woven, nonwoven, molded, extruded, cast, etc.), silicon, silicon oxide, and composites thereof. A support can be an array. In some embodiments, a support comprises an array. An array can comprise an ordered spatial arrangement of two or more discrete regions. An array can comprise anti-CDH17 antibodies or antigen-binding fragments thereof located at known or unknown discrete regions. Row and column arrangements of arrays can be selected due to the relative simplicity in making such arrangements. The spatial arrangement can, however, be essentially any form selected by the user, and optionally, in a pattern. Areas of an array may be any convenient shape, including circular, ellipsoid, oval, annular, or some other analogously curved shape, where the shape may, in certain embodiments, be a result of the particular method employed to produce the array.

In some embodiments, a support can be planar. In some instances, a support can be spherical. In some instances, a support can be a bead. In some instances, a support can be magnetic. In some embodiments, a magnetic support can comprise magnetite, maghemite, FePt, SrFe, iron, cobalt, nickel, chromium dioxide, ferrites, or mixtures thereof. In some embodiments, a support can be nonmagnetic. In some embodiments, the nonmagnetic support can comprise a polymer, metal, glass, alloy, mineral, or mixture thereof. In some instances, a nonmagnetic material can be a coating around a magnetic support. In some instances, a magnetic material may be distributed in the continuous phase of a magnetic material. In some embodiments, the support comprises magnetic and nonmagnetic materials. In some instances, a support can comprise a combination of a magnetic material and a nonmagnetic material. In some embodiments, an anti-CDH17 antibody or antigen-binding fragment thereof disclosed herein is directly or indirectly associated with a support disclosed herein.

### Computer Control System

The present disclosure provides computer control systems that are programmed to implement methods of the disclosure. In some embodiments, a computer system is programmed or otherwise configured to interface with an apparatus that is configured to detect CDH17 and/or binding of an anti-CDH17 antibody or antigen-binding fragment thereof disclosed herein to moiety. The computer system can be an electronic device of a user or a computer system that is remotely located with respect to the electronic device. The electronic device can be a mobile electronic device.

In some embodiments, a computer system includes a central processing unit (CPU, also “processor” and “computer processor” herein, which can be a single core or multi core processor, or a plurality of processors for parallel processing.

In some embodiments, a computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. In some embodiments, the memory, storage unit, interface and peripheral devices are in communication with the CPU through a communication bus, such as a motherboard. In some embodiments, the storage unit can be a data storage unit (or data repository) for storing data. In some embodiments, the computer system is operatively coupled to a computer network (“network”) with the aid of the communication interface. In some embodiments, the network can be the Internet, an internet and/or extranet, or an intranet and/or extranet that is in communication with the Internet. In some embodiments, the network is a telecommunication and/or data network. The network can include one or more computer servers, which can enable distributed computing, such as cloud computing. In some embodiments, the network, in some cases with the aid of the computer system can implement a peer-to-peer network, which may enable devices coupled to the computer system to behave as a client or a server. In some embodiments, the CPU executes a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback. In some embodiments, the CPU can be part of a circuit, such as an integrated circuit. One or more other components of the system can be included in the circuit. In some cases, the circuit is an application specific integrated circuit (ASIC). In some embodiments, the storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet. In some embodiments, the computer system communicates with one or more remote computer systems through the network. For instance, the computer system can communicate with a remote computer system of a user. Examples of remote computer systems include personal computers (e.g., portable PC), slate or tablet PC's telephones, Smart phones, or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory. In some embodiments, the code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

A machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution. In some embodiments, a computer system disclosed herein can include or be in communication with an electronic display that comprises a user interface (UI) for providing, for example, one or more results (immediate results or archived results from a previous experiment), one or more user inputs, reference values from a library or database, or a combination thereof. Examples of UIs include, without limitation, a graphical user interface (GUI) and web-based user interface.

Further, methods and systems of the present disclosure can be implemented by way of one or more algorithms. An algorithm can be implemented by way of software upon execution by the central processing unit. The algorithm can, for example, determine optimized conditions via supervised learning to optimize conditions such as a buffer type, a buffer concentration, a temperature, an incubation period, thresholds, diagnostic/prognostic indications for methods disclosed herein.

### Numbered Embodiments

1. Numbered embodiment 1 comprises an anti-CDH17 antibody or antigen-binding fragment thereof comprising (a) a HCDR1 having at least 80% sequence identity to the amino acid sequence set out in any one of SEQ ID NO: 41, (b) a HCDR2 having the amino acid sequence set out in any one of SEQ ID NOs: 43-60, and (c) a HCDR3 having at least 80% sequence identity to the amino acid sequence set out in any one of SEQ ID NOs: 61-75.
2. Numbered embodiment 2 comprises an anti-CDH17 antibody or antigen-binding fragment thereof comprising (a) a HCDR1 having at least 80% sequence identity to the amino acid sequence set out in any one of SEQ ID NO: 41, (b) a HCDR2 having at least 80% sequence identity to the amino acid sequence set out in any one of SEQ ID NOs: 42-60, and (c) a HCDR3 having the amino acid sequence set out in any one of SEQ ID NOs: 62-75.
3. Numbered embodiment 3 comprises the anti-CDH17 antibody or antigen-binding fragment thereof of numbered embodiments 1 or 2, wherein the anti-CDH17 antibody or antigen-binding fragment thereof comprises at least one of (a) a HCDR1 comprising the amino acid sequence set out in SEQ ID NO: 41, (b) a HCDR2 comprising the amino acid sequence set out in any one of SEQ ID NOs: 43-60 and (c) a HCDR3 comprising the amino acid sequence set out in any one of SEQ ID NOs: 62-75.
4. Numbered embodiment 4 comprises the anti-CDH17 antibody or antigen-binding fragment thereof of any one of numbered embodiments 1-3, wherein the anti-CDH17 antibody or antigen-binding fragment thereof has at least 90% sequence identity to an amino acid sequence set out in SEQ ID NO: 1.
5. Numbered embodiment 5 comprises the anti-CDH17 antibody or antigen-binding fragment thereof of any one of numbered embodiments 1-4, wherein the anti-CDH17 antibody or antigen-binding fragment thereof has at least 95% sequence identity to an amino acid sequence set out in SEQ ID NO: 1. 6. Numbered embodiment 6 comprises an anti-CDH17 antibody or antigen-binding fragment thereof comprising a HCDR1 as set out in SEQ ID NO: 41, a HCDR2 as set out in SEQ ID NO: 43, and/or a HCDR3 as set out in SEQ ID NO: 62.
7. Numbered embodiment 7 comprises the anti-CDH17 antibody or antigen-binding fragment thereof of numbered embodiment 6, wherein the anti-CDH17 antibody or antigen-binding fragment thereof has at least 80% sequence identity to the amino acid sequence set out in SEQ ID NO: 2.
8. Numbered embodiment 8 comprises an anti-CDH17 antibody or antigen-binding fragment thereof comprising a HCDR1 as set out in SEQ ID NO: 41, a HCDR2 as set out in SEQ ID NO: 44, and/or a HCDR3 as set out in SEQ ID NO: 63.
9. Numbered embodiment 9 comprises the anti-CDH17 antibody or antigen-binding fragment thereof of numbered embodiment 8, wherein the anti-CDH17 antibody or antigen-binding fragment thereof has at least 80% sequence identity to the amino acid sequence set out in SEQ ID NO: 3.
10. Numbered embodiment 10 comprises an anti-CDH17 antibody or antigen-binding fragment thereof comprising a HCDR1 as set out in SEQ ID NO: 41, a HCDR2 as set out in SEQ ID NO: 43, and/or a HCDR3 as set out in SEQ ID NO: 64.
11. Numbered embodiment 11 comprises the anti-CDH17 antibody or antigen-binding fragment thereof of numbered embodiment 10, wherein the anti-CDH17 antibody or antigen-binding fragment thereof has at least 80% sequence identity to the amino acid sequence set out in SEQ ID NO: 4.
12. Numbered embodiment 12 comprises an anti-CDH17 antibody or antigen-binding fragment thereof comprising a HCDR1 as set out in SEQ ID NO: 41, a HCDR2 as set out in SEQ ID NO: 45, and/or a HCDR3 as set out in SEQ ID NO: 65.
13. Numbered embodiment 13 comprises the anti-CDH17 antibody or antigen-binding fragment thereof of numbered embodiment 12, wherein the anti-CDH17 antibody or antigen-binding fragment thereof has at least 80% sequence identity to the amino acid sequence set out in SEQ ID NO: 5.
14. Numbered embodiment 14 comprises an anti-CDH17 antibody or antigen-binding fragment thereof comprising a HCDR1 as set out in SEQ ID NO: 41, a HCDR2 as set out in SEQ ID NO: 46, and/or a HCDR3 as set out in SEQ ID NO: 65.
15. Numbered embodiment 15 comprises the anti-CDH17 antibody or antigen-binding fragment thereof of numbered embodiment 14, wherein the anti-CDH17 antibody or antigen-binding fragment thereof has at least 80% sequence identity to the amino acid sequence set out in SEQ ID NO: 6.
16. Numbered embodiment 16 comprises an anti-CDH17 antibody or antigen-binding fragment thereof comprising a HCDR1 as set out in SEQ ID NO: 41, a HCDR2 as set out in SEQ ID NO: 47, and/or a HCDR3 as set out in SEQ ID NO: 72.
17. Numbered embodiment 17 comprises the anti-CDH17 antibody or antigen-binding fragment thereof of numbered embodiment 16, wherein the anti-CDH17 antibody or antigen-binding fragment thereof comprises an amino acid sequence identity to the amino acid sequence set out in SEQ ID NO: 109.
18. Numbered embodiment 18 comprises an anti-CDH17 antibody or antigen-binding fragment thereof comprising at least one amino acid substitutions at position 25-106 in an amino acid sequence as set out in SEQ ID NO: 1.
19. Numbered embodiment 19 comprises the anti-CDH17 antibody or antigen-binding fragment thereof of numbered embodiment 18, wherein the at least one amino acid substitutions comprise at least two, three, four, five, six, or seven amino acid substitutions.
20. Numbered embodiment 20 comprises the anti-CDH17 antibody or antigen-binding fragment thereof of numbered embodiment 18 or 19, wherein the at least one amino acid substitutions comprise a substitution of an amino acid with I, Y, D, R, N, S, A, E, T, F, H, V, L, or K.
21. Numbered embodiment 21 comprises the anti-CDH17 antibody or antigen-binding fragment thereof of any one of numbered embodiments 18-20, wherein the at least one amino acid substitutions comprise R25I, Q37Y, A38Y, S50D, N52R, E54N, E54I, N55Y, N55R, A56S, S59R, S59A, D60E, S61D, S61T, S61F, M63H, T67V, H98Y, Y98L, G100V, G100N, G100K, G100T, R102K, S106L, S106K, or S106A.
22. Numbered embodiment 22 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X, is S or L.
23. Numbered embodiment 23 comprises an anti-CDH17 antibody or antigen-binding fragment thereof, comprising (a) a HCDR1 having an amino acid sequence YDMG (SEQ ID NO: 41); (b) a HCDR2 having an amino acid sequence LLSWX₁GNNAEYX₃DX₄VX₅G (SEQ ID NO: 81), wherein X₁ is N or R, X₃ is S or R, X₄ is S, D, or F, and X₅ is M or H; and (c) a HCDR3 having an amino acid sequence TX₆GX-AX: SVRX, (SEQ ID NO: 80), wherein X₆ is H or Y, X₇ is G, T, V, K or N, X₈ is R or K, and X₉ is S or L.
24. Numbered embodiment 24 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYRDX₄VX₅G (SEQ ID NO: 82), wherein X₁ is N or R, X₂ is E or N, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is G, T, V, K or N, X₈ is R or K, and X₉ is S or L.
25. Numbered embodiment 25 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is D or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L.
26. Numbered embodiment 26 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VHG (SEQ ID NO: 84), wherein X₁ is N or R, X₂ is E or N, X₃ is S or R, and X₄ is S, D, or F; and (c) a HCDR3 having an amino acid sequence TX₆GX₇AX₈SVRX, (SEQ ID NO: 80), wherein X₆ is H or Y, X₇ is G, T, V, K or N, X₈ is R or K, and X₉ is S or L.
27. Numbered embodiment 27 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TYGX-AX₈SVRX₉ (SEQ ID NO: 85), wherein X₇ is G, T, V, K or N, X₈ is R or K, and X₉ is S or L.
28. Numbered embodiment 28 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRX₉ (SEQ ID NO: 80), wherein X₆ is H or Y, X₇ is T, V, K or N, X₈ is R or K, and X₉ is S or L. 29. Numbered embodiment 29 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AK SVRX₉ (SEQ ID NO: 86), wherein X₆ is H or Y, X₇ is G, T, V, K or N, and X₉ is S or L.
30. Numbered embodiment 30 comprises an anti-CDH17 antibody or antigen-binding fragment thereof comprising (a) a HCDR1 having an amino acid sequence YDMG (SEQ ID NO: 41); (b) a HCDR2 having an amino acid sequence LLSWX₁GX₂NAEYX₃DX₄VX₅G (SEQ ID NO: 83), wherein X₁ is N or R, X₂ is E or N, X₃ is S or R, X₄ is S, D, or F, and X₅ is M or H; and (c) a HCDR3 having an amino acid sequence TX₆GX-AX₈SVRL (SEQ ID NO: 87), wherein X₆ is H or Y, X₇ is G, T, V, K or N, and X₈ is R or K.
31. Numbered embodiment 31 comprises the anti-CDH17 antibody or antigen-binding fragment thereof of any one of numbered embodiments 22-30, wherein the anti-CDH17 antibody or antigen-binding fragment thereof comprises a T67V mutation in an amino acid sequence as set out in SEQ ID NO: 1.
32. Numbered embodiment 32 comprises an anti-CDH17 antibody or antigen-binding fragment thereof comprising (a) a HCDR1 having an amino acid sequence YDMG (SEQ ID NO: 41); (b) a HCDR2 having an amino acid sequence LLDWX₁₁GX₁₂X₁₃X₁₄EYX₁₅X₁₆X₁₇VX₁₈G (SEQ ID NO: 88), wherein X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.
33. Numbered embodiment 33 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WRGX₁₂X₁₃X₁₄EYX₁₅X₁₆X₁₇VX₁₈G (SEQ ID NO: 90), wherein X₁₀ is S or D, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.
34. Numbered embodiment 34 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 91), wherein X₁₀ is S or D, X₁₁ is N or R, X₁₂ is N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.
35. Numbered embodiment 35 comprises an anti-CDH17 antibody or antigen-binding fragment thereof comprising (a) a HCDR1 having an amino acid sequence YDMG (SEQ ID NO: 41); (b) having a HCDR2 an amino acid sequence LLX₁₀WX₁₁GX₁₂YX₁₄EYX₁₅X₁₆X₁₇VX₁₈G (SEQ ID NO: 92), wherein X₁₀ is S or D, Xu is N or R, X₁₂ is E, N, I or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.
36. Numbered embodiment 36 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃SEYX₁₅X₁₆X₁₇VX₁₈G (SEQ ID NO: 93), wherein X₁₀ is S or D, X₁₁ is N or R, X₁₂ is E, N, I or Y, X₁₃ is N or Y, X₁₅ is S, R or A, X₁₆ is D or E, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.
37. Numbered embodiment 37 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.
38. Numbered embodiment 38 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅EX₁₇VX₁₈G (SEQ ID NO: 95), wherein X₁₀ is S or D, X₁₁ is N or R, X₁₂ is E, N, I or Y, X₁₃ is N or Y, X₁₄ is A or S, X₅ is S, R or A, X₁₇ is S, D, F or T, and X₁₈ is M or H; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A.
39. Numbered embodiment 39 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is D, F or T, and X₁₈ is M or H; and (c) a HCDR3 having an amino acid sequence TX₁₉GX₂₀AX₂₁SVRX₂₂ (SEQ ID NO: 96), wherein X₁₉ is H or Y, X₂₀ is G, T, V, K or N, X₂₁ is R or K, and X₂₂ is S, L, K or A.
40. Numbered embodiment 40 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VHG (SEQ ID NO: 97), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T; and (c) a HCDR3 having an amino acid sequence TX₁₉GX₂₀AX₂₁SVRX₂₂ (SEQ ID NO: 89), wherein X₁₉ is H or Y, X₂₀ is G, T, V, K or N, X₂₁ is R or K, and X₂₂ is S, L, K or A. 41. Numbered embodiment 41 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TYGX₂₀AX₂₁SVRX₂₂ (SEQ ID NO: 98), wherein X₂₀ is G, T, V, K or N, X₂₁ is R or K, and X₂₂ is S, L, K or A.
42. Numbered embodiment 42 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is T, V, K or N, X₂₁ is R or K, and X₂₂ is S, L, K or A.
43. Numbered embodiment 43 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, X₁₁ is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AKSVRX₂₂ (SEQ ID NO: 99), wherein X₁₉ is H or Y, X₂₀ is G, T, V, K or N, and X₂₂ is S, L, K or A.
44. Numbered embodiment 44 comprises an anti-CDH17 antibody or antigen-binding fragment thereof comprising (a) a HCDR1 having an amino acid sequence YDMG (SEQ ID NO: 41); (b) a HCDR2 having an amino acid sequence LLX₁₀WX₁₁GX₁₂X₁₃X₁₄EYX₁₅X₁₆X₁₇VX₁₈G (SEQ ID NO: 94), wherein X₁₀ is S or D, Xu is N or R, X₁₂ is E, N, I or Y, X₁₃ is N or Y, X₁₄ is A or S, X₁₅ is S, R or A, X₁₆ is D or E, X₁₇ is S, D, F or T, and X₁₈ is M or H; and (c) a HCDR3 having an amino acid sequence TX₁₉ GX₂₀AX₂₁SVRX₂₂ (SEQ ID NO: 89), wherein X₁₉ is H or Y, X₂₀ is G, T, V, K or N, X₂₁ is R or K, and X₂₂ is L, K or A.
45. Numbered embodiment 45 comprises the anti-CDH17 antibody or antigen-binding fragment thereof of any one of numbered embodiments 32-44, wherein the anti-CDH17 antibody or antigen-binding fragment thereof comprises Q37Y, A38Y, R52I and/or T67V in an amino acid sequence as set out in SEQ ID NO: 1.
46. Numbered embodiment 46 comprises the anti-CDH17 antibody or antigen-binding fragment thereof of any one of numbered embodiments 1-45, wherein the anti-CDH17 antibody or antigen-binding fragment thereof comprises an amino acid sequence having at least 90% sequence identity to the amino acid sequence set out in any one of SEQ ID NO: 2-40 and 109.
47. Numbered embodiment 47 comprises the anti-CDH17 antibody or antigen-binding fragment thereof of any one of numbered embodiments 1-46, wherein the anti-CDH17 antibody or antigen-binding fragment thereof comprises an amino acid sequence having the amino acid sequence set out in any one of SEQ ID NO: 2-40 and 109.
48. Numbered embodiment 48 comprises an anti-CDH17 antibody or antigen-binding fragment thereof, wherein the anti-CDH17 antibody or antigen-binding fragment thereof has an increased binding affinity to CDH17 compared to a binding affinity of an amino acid sequence as set out in SEQ ID NO: 1 to CDH17.
49. Numbered embodiment 49 comprises the anti-CDH17 antibody or antigen-binding fragment thereof of numbered embodiment 48, wherein the binding affinity of the anti-CDH17 antibody or antigen-binding fragment thereof to CDH17 is at least 3-fold greater than the binding affinity of an amino acid sequence as set out in SEQ ID NO: 1 to CDH17.
50. Numbered embodiment 50 comprises the anti-CDH17 antibody or antigen-binding fragment thereof of numbered embodiment 48, wherein the binding affinity of the anti-CDH17 antibody or antigen-binding fragment thereof to CDH17 is at least 5-fold greater than the binding affinity of an amino acid sequence as set out in SEQ ID NO: 1 to CDH17.
51. Numbered embodiment 51 comprises the anti-CDH17 antibody or antigen-binding fragment thereof of numbered embodiments 48-50, wherein the binding affinity of the anti-CDH17 antibody or antigen-binding fragment thereof to CDH17 is at least 10-fold greater than the binding affinity of an amino acid sequence as set out in SEQ ID NO: 1 to CDH17.
52. Numbered embodiment 52 comprises the anti-CDH17 antibody or antigen-binding fragment thereof of any one of numbered embodiments 48-51, wherein the binding affinity of the anti-CDH17 antibody or antigen-binding fragment thereof to CDH17 is at least 15-fold greater than the binding affinity of an amino acid sequence as set out in SEQ ID NO: 1 to CDH17.
53. Numbered embodiment 53 comprises the anti-CDH17 antibody or antigen-binding fragment thereof of any one of numbered embodiments 48-52, wherein the anti-CDH17 antibody or antigen-binding fragment thereof has a KD for binding to CDH17 of less than 1.0×10⁻⁸ M.
54. Numbered embodiment 54 comprises the anti-CDH17 antibody or antigen-binding fragment thereof of any one of numbered embodiments 48-53, wherein the anti-CDH17 antibody or antigen-binding fragment thereof has a KD for binding to CDH17 of less than 5.0×10⁻⁹ M.
55. Numbered embodiment 55 comprises the anti-CDH17 antibody or antigen-binding fragment thereof of any one of numbered embodiments 48-54, wherein the anti-CDH17 antibody or antigen-binding fragment thereof has a KD for binding to CDH17 of less than 3.0×10⁻⁹ M.
56. Numbered embodiment 56 comprises the anti-CDH17 antibody or antigen-binding fragment thereof of any one of numbered embodiments 48-55, wherein the anti-CDH17 antibody or antigen-binding fragment thereof inhibits CDH17 activity with an IC₅₀ of less than 1.0×10⁻⁹ M.
57. Numbered embodiment 57 comprises the anti-CDH17 antibody or antigen-binding fragment thereof of any one of numbered embodiments 48-56, wherein the anti-CDH17 antibody or antigen-binding fragment thereof inhibits CDH17 activity with an IC₅₀ of less than 100.0×10⁻¹² M.
58. Numbered embodiment 58 comprises the anti-CDH17 antibody or antigen-binding fragment thereof of any one of numbered embodiments 48-57, wherein the anti-CDH17 antibody or antigen-binding fragment thereof inhibits CDH17 activity with an IC₅₀ of less than 50.0×10⁻¹² M.
59. Numbered embodiment 59 comprises the anti-CDH17 antibody or antigen-binding fragment thereof of any one of numbered embodiments 48-58, wherein the IC₅₀ of the anti-CDH17 antibody or antigen-binding fragment thereof to CDH17 is at least 10-fold lesser than the IC₅₀ of an amino acid sequence as set out in SEQ ID NO: 1 to CDH17.
60. Numbered embodiment 60 comprises the anti-CDH17 antibody or antigen-binding fragment thereof of any one of numbered embodiments 48-59, wherein the IC₅₀ of the anti-CDH17 antibody or antigen-binding fragment thereof to CDH17 is at least 30-fold lesser than the IC₅₀ of an amino acid sequence as set out in SEQ ID NO: 1 to CDH17.
61. Numbered embodiment 61 comprises an anti-CDH17 antibody or antigen-binding fragment thereof, wherein the anti-CDH17 antibody or antigen-binding fragment thereof has increased stability compared to stability of an amino acid sequence as set out in SEQ ID NO: 1.
62. Numbered embodiment 62 comprises the anti-CDH17 antibody or antigen-binding fragment thereof of numbered embodiment 61, wherein the stability of the anti-CDH17 antibody or antigen-binding fragment thereof to CDH17 is measured by melting temperature (Tm).
63. Numbered embodiment 63 comprises the anti-CDH17 antibody or antigen-binding fragment thereof of numbered embodiment 61 or 62, wherein the anti-CDH17 antibody or antigen-binding fragment thereof has a Tm of less than 70° C.
64. Numbered embodiment 64 comprises the anti-CDH17 antibody or antigen-binding fragment thereof of any one of numbered embodiments 61-63, wherein the anti-CDH17 antibody or antigen-binding fragment thereof has a Tm of less than 60° C.
65. Numbered embodiment 65 comprises the anti-CDH17 antibody or antigen-binding fragment thereof of any one of numbered embodiments 61-64, wherein the anti-CDH17 antibody or antigen-binding fragment thereof has a Tm of less than 50° C.
66. Numbered embodiment 66 comprises an anti-CDH17 antibody or antigen-binding fragment thereof, wherein the anti-CDH17 antibody or antigen-binding fragment thereof has a higher purity compared to a purity of an amino acid sequence as set out in SEQ ID NO: 1.
67. Numbered embodiment 67 comprises the anti-CDH17 antibody or antigen-binding fragment thereof of numbered embodiment 66, wherein the anti-CDH17 antibody or antigen-binding fragment thereof has a purity of more than 96%.
68. Numbered embodiment 68 comprises the anti-CDH17 antibody or antigen-binding fragment thereof of numbered embodiment 66 or 67, wherein the anti-CDH17 antibody or antigen-binding fragment thereof has a purity of more than 97%.
69. Numbered embodiment 69 comprises the anti-CDH17 antibody or antigen-binding fragment thereof of any one of numbered embodiments 66-68, wherein the anti-CDH17 antibody or antigen-binding fragment thereof has a purity of more than 98%.
70. Numbered embodiment 70 comprises the anti-CDH17 antibody or antigen-binding fragment thereof of any one of numbered embodiments 66-69, wherein the anti-CDH17 antibody or antigen-binding fragment thereof has a purity of more than 99%.
71. Numbered embodiment 71 comprises the anti-CDH17 antibody or antigen-binding fragment thereof of any one of numbered embodiments 66-70, wherein the anti-CDH17 antibody or antigen-binding fragment thereof has a purity of 100%.
72. Numbered embodiment 72 comprises the anti-CDH17 antibody or antigen-binding fragment thereof of any one of numbered embodiments 66-71, wherein purity is measured via capillary gel electrophoresis using sodium dodecyl sulfate (CE-SDS).
73. Numbered embodiment 73 comprises the anti-CDH17 antibody or antigen-binding fragment thereof of any one of numbered embodiments 48-72, wherein the anti-CDH17 antibody or antigen-binding fragment thereof is the anti-CDH17 antibody or antigen-binding fragment thereof of any one of numbered embodiments 1-47.
74. Numbered embodiment 74 comprises a multi-specific binding molecule comprising a first binding domain and a second binding domain, wherein the first binding domain comprises the anti-CDH17 antibody or antigen-binding fragment thereof of any one of numbered embodiments 1-73.
75. Numbered embodiment 75 comprises the multi-specific binding molecule of numbered embodiment 74, wherein the first binding domain and/or the second binding domain comprises a single chain variable fragment (scFv), a Fab fragment, or a combination thereof.
76. Numbered embodiment 76 comprises the multi-specific binding molecule of numbered embodiment 74 or 75, wherein the first binding domain binds to an antigen expressed on the surface of a cancer cell.
77. Numbered embodiment 77 comprises the multi-specific binding molecule of any one of numbered embodiments 74-76, wherein the second binding domain binds to an antigen expressed on the surface of an immune cell.
78. Numbered embodiment 78 comprises the multi-specific binding molecule of numbered embodiments 74-77, wherein the antigen expressed on the surface of an immune cell comprises CD2, CD3D, CD3E, CD247, CD27, C28, IL7R, CD45, CD69, ICOS, IL2RB, SELL, CD137, CD16, CD3Y, CD28, 4-1BB or CD134.
79. Numbered embodiment 79 comprises the multi-specific binding molecule of any one of numbered embodiments 74-78, further comprising a Fc domain.
80. Numbered embodiment 80 comprises the multi-specific binding molecule of any one of numbered embodiments 74-79, wherein the Fc domain is derived from IgA, IgD, IgE, IgG, or IgM.
81. Numbered embodiment 81 comprises the multi-specific binding molecule of any one of numbered embodiments 74-80, comprising a linker peptide.
82. Numbered embodiment 82 comprises the multi-specific binding molecule of any one of numbered embodiments 74-81, wherein a linker peptide comprises about 2-20 amino acids.
83. Numbered embodiment 83 comprises the multi-specific binding molecule of any one of numbered embodiments 74-82, wherein a linker peptide comprises (G₄S)ₙ, (SG₄)ₙ, G₄(SG₄)ₙ or G₂(SG₂)ₙ, wherein n is selected from 1 to 10.
84. Numbered embodiment 84 comprises the multi-specific binding molecule of any one of numbered embodiments 74-83, wherein the linker peptide comprises GGGGSGGGGS (SEQ ID NO: 100) or GGGGS (SEQ ID NO: 101).
85. Numbered embodiment 85 comprises the multi-specific binding molecule of any one of numbered embodiments 74-84, wherein the linker peptide links the anti-CDH17 antibody or antigen-binding fragment thereof of any one of numbered embodiments 1-73 to an Fc domain.
86. Numbered embodiment 86 comprises a protein comprising the anti-CDH17 antibody or antigen-binding fragment thereof of any one of numbered embodiments 1-73 conjugated to a cytotoxic agent, and/or a Fc domain.
87. Numbered embodiment 87 comprises the protein of numbered embodiment 86, wherein the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB).
88. Numbered embodiment 88 comprises the protein of numbered embodiment 87, wherein the cytotoxic agent is MMAE.
89. Numbered embodiment 89 comprises the protein of numbered embodiment 88, further comprising a linker.
90. Numbered embodiment 90 comprises the protein of numbered embodiment 89, wherein the linker comprises a bond, a cleavable linker, and/or a non-cleavable linker.
91. Numbered embodiment 91 comprises the protein of numbered embodiment 90, wherein the linker comprises an attachment group, a protease-cleavable linker, and/or a spacer.
92. Numbered embodiment 92 comprises the protein of numbered embodiment 91, wherein the attachment group comprises maleimide and/or caproic acid.
93. Numbered embodiment 93 comprises the protein of numbered embodiment 92, wherein the protease-cleavable linker is a cathepsin-cleavable linker.
94. Numbered embodiment 94 comprises the protein of numbered embodiment 93, wherein the cathepsin-cleavable linker is a valine-citrulline linker.
95. Numbered embodiment 95 comprises the protein of numbered embodiment 94, further comprising a spacer.
96. Numbered embodiment 96 comprises the protein of numbered embodiment 95, wherein the spacer is PABC.
97. Numbered embodiment 97 comprises the protein of numbered embodiment 87, having a drug antibody ratio (DAR) of about 2, 4, or 8.
98. Numbered embodiment 98 comprises the protein of numbered embodiment 87, wherein DAR is about 4.
99. Numbered embodiment 99 comprises the protein of numbered embodiments 86-98, wherein the Fc domain is derived from IgA, IgD, IgE, IgG, or IgM.
100. Numbered embodiment 100 comprises a conjugate comprising Formula (I):

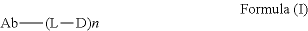

wherein Ab comprises an anti-CDH17 antibody or antigen-binding fragment thereof, wherein the anti-CDH17 antibody or antigen-binding fragment thereof comprises (a) a HCDR1 having at least 80% sequence identity to the amino acid sequence set out in of SEQ ID NO: 41, (b) a HCDR2 having at least 80% sequence identity to the amino acid sequence set out in any one of SEQ ID NOs: 43-60, and (c) a HCDR3 having at least 80% sequence identity to the amino acid sequence set out in any one of SEQ ID NOs: 61-75;
   L is a linker;
   D is a cytotoxic agent; and
   n is a drug antibody ratio, wherein the drug antibody ratio is from about 1 to about 8.
101. Numbered embodiment 101 comprises the conjugate of numbered embodiment 100, wherein the anti-CDH17 antibody or antigen-binding fragment thereof comprises (a) a HCDR1 having an amino acid sequence as set out in SEQ ID NO: 41, (b) a HCDR2 having an amino acid sequence as set out in SEQ ID NO: 47, and (c) a HCDR3 having an amino acid sequence as set out in SEQ ID NO: 72.
102. Numbered embodiment 102 comprises the conjugate of numbered embodiment 101, wherein the anti-CDH17 antibody or antigen-binding fragment thereof comprises an amino acid sequence having at least 80% sequence identity to the amino acid sequence set out in any one of SEQ ID NO: 2-40 and 109.
103. Numbered embodiment 103 comprises the conjugate of numbered embodiment 102, wherein the anti-CDH17 antibody or antigen-binding fragment thereof comprises an amino acid sequence having an amino acid sequence as set out in SEQ ID NO: 109
104. Numbered embodiment 104 comprises the conjugate of numbered embodiment 103, wherein the anti-CDH17 antibody or antigen-binding fragment thereof further comprises a Fc domain.
105. Numbered embodiment 105 comprises the conjugate of numbered embodiment 104, wherein the Fc domain is derived from IgA, IgD, IgE, IgG, or IgM.
106. Numbered embodiment 106 comprises the conjugate of any one of numbered embodiments 100-105, wherein the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB).
107. Numbered embodiment 107 comprises the conjugate of numbered embodiment 106, wherein the cytotoxic agent is MMAE.
108. Numbered embodiment 108 comprises the conjugate of numbered embodiment 107, wherein the linker comprises an attachment group, a protease-cleavable linker, and/or a spacer.
109. Numbered embodiment 109 comprises the conjugate of numbered embodiment 108, wherein the attachment group comprises maleimide and/or caproic acid.
110. Numbered embodiment 110 comprises the conjugate of numbered embodiment 109, wherein the protease-cleavable linker is a cathepsin-cleavable linker.
111. Numbered embodiment 111 comprises the conjugate of numbered embodiment 110, wherein the cathepsin-cleavable linker is a valine-citrulline linker.
112. Numbered embodiment 112 comprises the conjugate of numbered embodiment 111, further comprising a spacer.
113. Numbered embodiment 113 comprises the conjugate of numbered embodiment 112, wherein the spacer is PABC.
114. Numbered embodiment 114 comprises the conjugate of numbered embodiment 113, wherein n is about 4.
115. Numbered embodiment 115 comprises the anti-CDH17 antibody or antigen-binding fragment thereof of any one of numbered embodiments 1-73, wherein the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof.
116. Numbered embodiment 116 comprises the anti-CDH17 antibody or antigen-binding fragment thereof of any one of numbered embodiments 1-73, wherein the anti-CDH17 antibody or antigen-binding fragment thereof is a scFv antibody.
117. Numbered embodiment 117 comprises the anti-CDH17 antibody or antigen-binding fragment thereof of any one of numbered embodiments 1-73, wherein the anti-CDH17 antibody or antigen-binding fragment thereof is a VHH antibody.
118. Numbered embodiment 118 comprises the anti-CDH17 antibody or antigen-binding fragment thereof of any one of numbered embodiments 1-73, wherein the anti-CDH17 antibody or antigen-binding fragment thereof is humanized.
119. Numbered embodiment 119 comprises the anti-CDH17 antibody or antigen-binding fragment thereof of any one of numbered embodiments 1-73, wherein the anti-CDH17 antibody or antigen-binding fragment thereof is nonhuman antibody.
120. Numbered embodiment 120 comprises the anti-CDH17 antibody or antigen-binding fragment thereof of any one of numbered embodiments 1-73, wherein the anti-CDH17 antibody or antigen-binding fragment thereof is a human antibody.
121. Numbered embodiment 121 comprises a nucleic acid encoding the anti-CDH17 antibody or antigen-binding fragment thereof of any one of numbered embodiments 1-73, the multi-specific binding molecule of any one of numbered embodiments 74-85, the protein of any one of numbered embodiments 86-99, and/or the conjugate of any one of numbered embodiments 100-114.
122. Numbered embodiment 122 comprises the nucleic acid of numbered embodiment 121, wherein the nucleic acid comprises DNA, RNA, ssRNA, siRNA, microRNA, or mRNA.
123. Numbered embodiment 123 comprises an expression vector comprising the nucleic acid of numbered embodiment 121 or 122.
124. Numbered embodiment 124 comprises a host cell comprising the nucleic acid of numbered embodiment 121 or 122 or the expression vector of numbered embodiment 123.
125. Numbered embodiment 125 comprises a method of producing the anti-CDH17 antibody or antigen-binding fragment thereof of any one of numbered embodiments 1-73, the multi-specific binding molecule of any one of numbered embodiments 74-85, the protein of any one of numbered embodiments 86-99, the conjugate of any one of numbered embodiments 100-114, the method comprising maintaining the host cell of numbered embodiment 124 in a medium to produce the anti-CDH17 antibody or antigen-binding fragment thereof of any one of numbered embodiments 1-73, the multi-specific binding molecule of any one of numbered embodiments 74-85, the protein of any one of numbered embodiments 86-98 and isolating or purifying the anti-CDH17 antibody or antigen-binding fragment thereof of any one of numbered embodiments 1-73, the multi-specific binding molecule of any one of numbered embodiments 74-85, the protein of any one of numbered embodiments 86-98, and/or the conjugate of any one of numbered embodiments 100-114 produced by the host cell.
126. Numbered embodiment 126 comprises a pharmaceutical composition for treating cancer, comprising the anti-CDH17 antibody or antigen-binding fragment thereof of any one of numbered embodiments 1-73, the multi-specific binding molecule of any one of numbered embodiments 74-85, the protein of any one of numbered embodiments 86-98, the conjugate of any one of numbered embodiments 100-114, the nucleic acid of numbered embodiment 121 or 122, the expression vector of numbered embodiment 123 or the host cell of numbered embodiment 124 and a pharmaceutically acceptable carrier or excipient.
127. Numbered embodiment 127 comprises the pharmaceutical composition of numbered embodiment 126, wherein the pharmaceutical composition further comprises at least one additional therapeutic agent.
128. Numbered embodiment 128 comprises the anti-CDH17 antibody or antigen-binding fragment thereof of any one of numbered embodiments 1-73, the multi-specific binding molecule of any one of numbered embodiments 74-85, the protein of any one of numbered embodiments 86-98, the conjugate of any one of numbered embodiments 100-114, the nucleic acid of numbered embodiment 121 or 122, the expression vector of numbered embodiment 123, the host cell of numbered embodiment 124, the method of numbered embodiment 125, or the pharmaceutical composition of numbered embodiment 126 or 127, for use in a therapy.
129. Numbered embodiment 129 comprises the anti-CDH17 antibody or antigen-binding fragment thereof of any one of numbered embodiments 1-73, the multi-specific binding molecule of any one of numbered embodiments 74-85, the protein of any one of numbered embodiments 86-98, the conjugate of any one of numbered embodiments 100-114, the nucleic acid of numbered embodiment 121 or 122, the expression vector of numbered embodiment 123, the host cell of numbered embodiment 124, the method of numbered embodiment 125, or the pharmaceutical composition of numbered embodiment 126 or 127, for use in a therapy, wherein the therapy is an antibody monotherapy, antibody-drug conjugate (ADC) therapy, T cell-engaging immunotherapy, or CAR-T therapy.
130. Numbered embodiment 130 comprises a method for treating cancer in a subject in need thereof comprising administering to the subject a therapeutically effective amount of the anti-CDH17 antibody or antigen-binding fragment thereof of any one of numbered embodiments 1-73, the multi-specific binding molecule of any one of numbered embodiments 74-85, the protein of any one of numbered embodiments 86-98, the conjugate of any one of numbered embodiments 100-114, the nucleic acid of numbered embodiment 121 or 122, the expression vector of numbered embodiment 123, the host cell of numbered embodiment 124, or the pharmaceutical composition of numbered embodiment 126 or 127.
131. Numbered embodiment 131 comprises a use of the anti-CDH17 antibody or antigen-binding fragment thereof of any one of numbered embodiments 1-73, the multi-specific binding molecule of any one of numbered embodiments 74-85, the protein of any one of numbered embodiments 86-98, the conjugate of any one of numbered embodiments 100-114, the nucleic acid of numbered embodiment 121 or 122, the expression vector of numbered embodiment 123, the host cell of numbered embodiment 124, or the pharmaceutical composition of numbered embodiment 126 or 127, in the manufacture of a medicament for treatment of cancer.
132. Numbered embodiment 132 comprises the use of numbered embodiment 131, wherein the cancer is gastric, pancreatic, neuroendocrine, or colorectal cancer.
133. Numbered embodiment 133 comprises the use of the anti-CDH17 antibody or antigen-binding fragment thereof of any one of numbered embodiments 1-73, the multi-specific binding molecule of any one of numbered embodiments 74-85, the protein of any one of numbered embodiments 86-98, the conjugate of any one of numbered embodiments 100-114, the nucleic acid of numbered embodiment 121 or 122, the expression vector of numbered embodiment 123, the host cell of numbered embodiment 124, or the pharmaceutical composition of numbered embodiment 126 or 127, for diagnosis of cancer.
134. Numbered embodiment 134 comprises the use of numbered embodiment 133, wherein the cancer is gastric, pancreatic, neuroendocrine, or colorectal cancer.
135. Numbered embodiment 135 comprises a kit comprising the anti-CDH17 antibody or antigen-binding fragment thereof of any one of numbered embodiments 1-73, the multi-specific binding molecule of any one of numbered embodiments 74-85, the protein of any one of numbered embodiments 86-98, the conjugate of any one of numbered embodiments 100-114, the nucleic acid of numbered embodiment 121 or 122, the expression vector of numbered embodiment 123, the host cell of numbered embodiment 124, or the pharmaceutical composition of numbered embodiment 126 or 127, and instructions for use.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1. Expression of CDH17 x CD3 Bispecific T Cell Antibodies and Validation of Purity and Activity

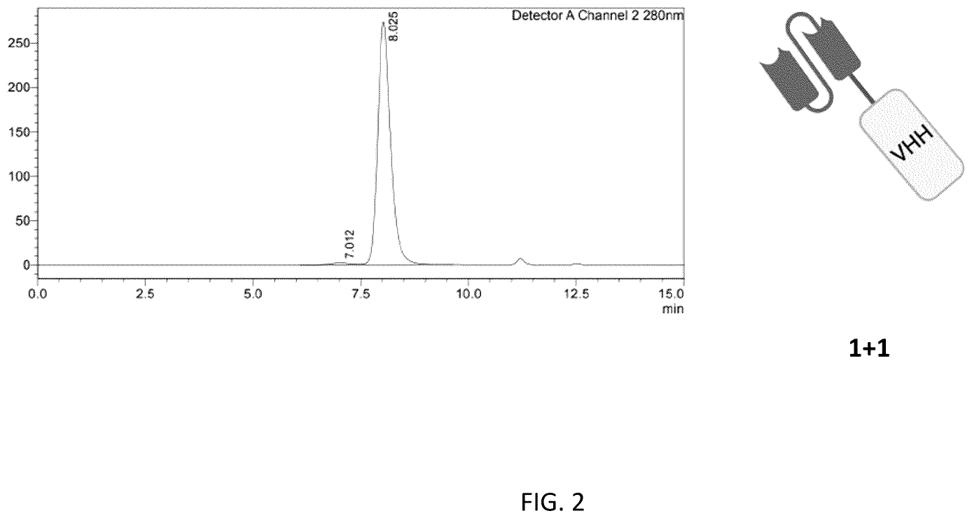

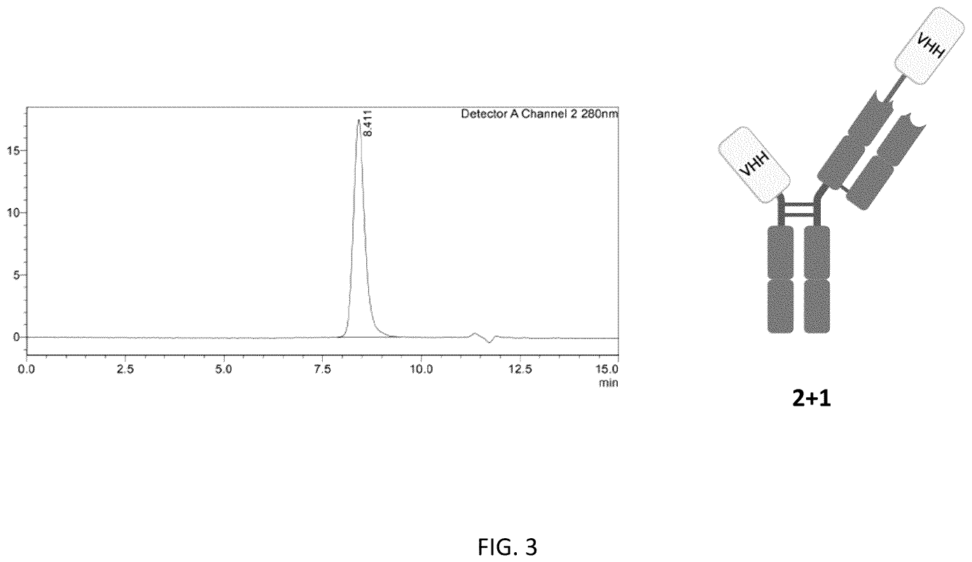

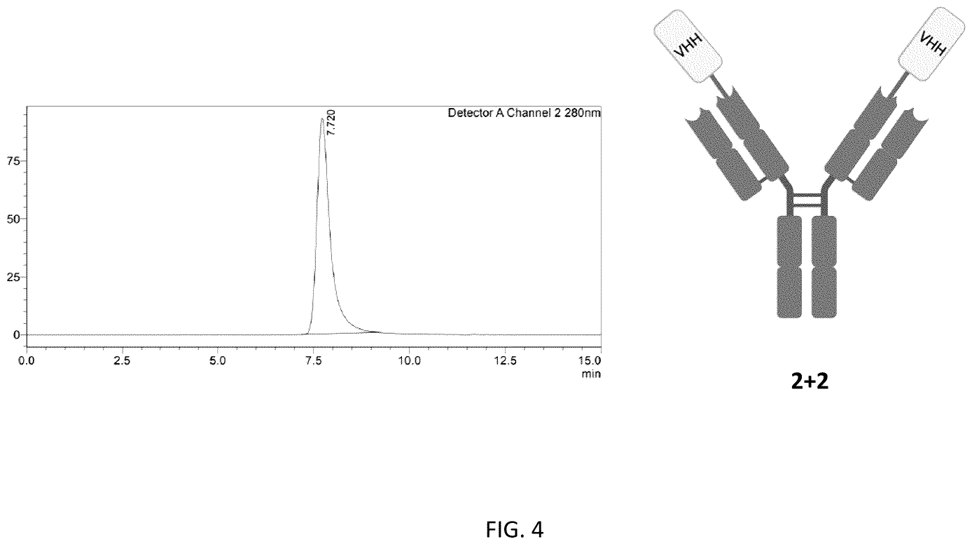

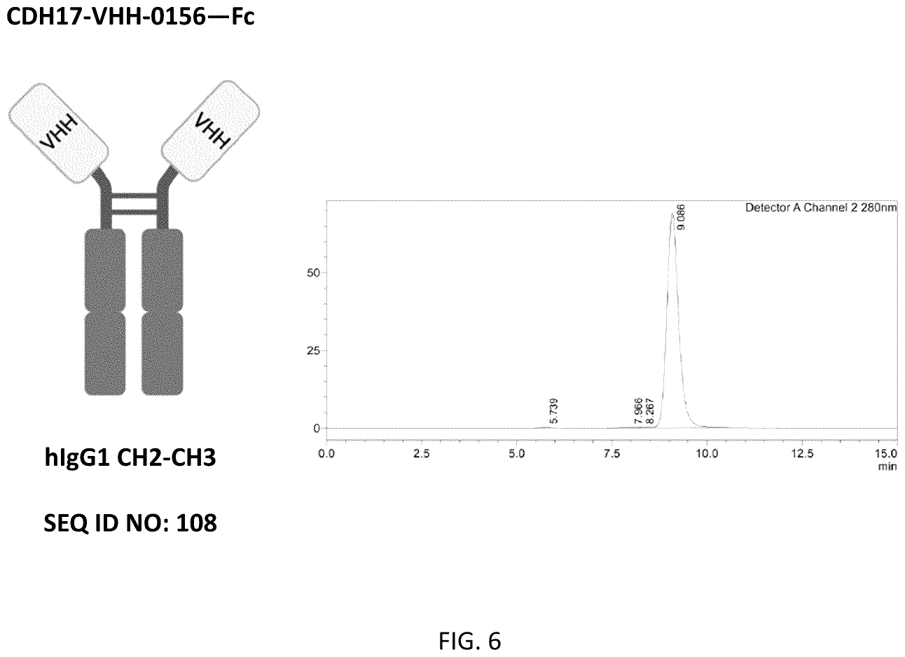

CDH17 x CD3 Bispecific T cell antibodies were cloned, expressed, and purified using standard methods. Briefly, antibody coding sequences were cloned into a suitable DNA vector for secretory protein expression in mammalian systems. These constructs were transiently transfected into CHO cells, and transfected cells were cultured for 5 days under standard conditions. Expressed antibodies containing a Fc homodimer were isolated from crude culture supernatants by Protein A column chromatography and elution and buffer exchanged into PBS. Post-purification purity was assessed by analytical SEC-HPLC. For asymmetric bispecific proteins containing knob-in-hole Fc heterodimers, a subsequent Protein L purification step was used to isolate the desired heterodimer species away from homodimer impurities. For hexa-histidine-tagged antibodies lacking Fc domains, standard metal affinity chromatography was used. Multi-specific post-purification yields were >10 mg/L, and VHH-Fc fusion protein yields exceeded 450 mg/L. FIG. 2 illustrates the high purity by HPLC of CDH17 x CD3 Bispecific T cell engager constructs lacking an Fc domain (1+1 geometry). FIG. 3 illustrates the high purity by HPLC of asymmetric CDH17 x CD3 Bispecific T cell engager constructs with an Fc domain (2+1 geometry). FIG. 4 illustrates the high purity by HPLC of Symmetric CDH17 x CD3 Bispecific T cell engager constructs with an Fc domain (2+2 geometry). FIG. 6 illustrates the high purity by HPLC of anti-CDH17 VHH-Fc fusion protein.

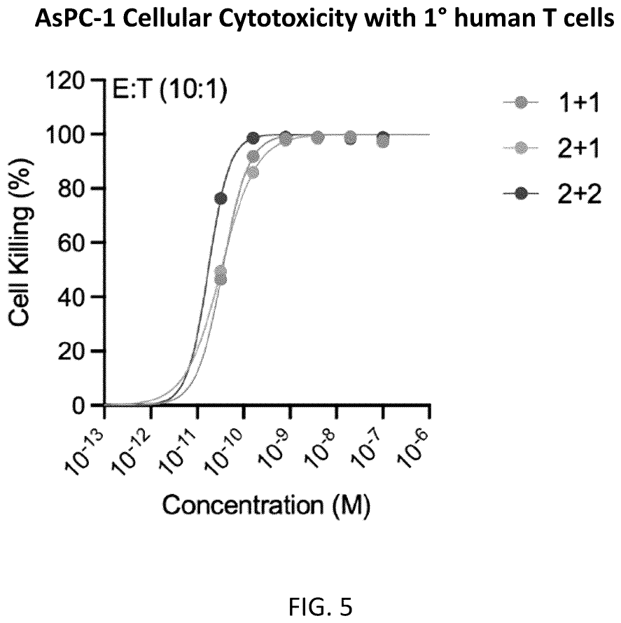

Cytotoxicity of antibodies to CDH17+ cells was determined by a T cell dependent cellular cytotoxicity (TDCC) assay (FIG. 5). Prior to the experiment, primary Human Pan-T cells from a single donor were thawed and rested overnight at 37° C., and AsPC-1 pancreatic adenocarcinoma cells were cultured to confluence using standard methods. 10,000 adherent target cells were pre-plated overnight prior to the experiment to ensure adequate adhesion. Subsequently, 100,000 rested T cells were added along with serial dilutions of antibodies. After 72 h of co-culture, supernatants and suspension cells were removed, and adherent cell monolayers were washed several times with PBS. Viable adherent cells were quantitated by addition of Cell Titer-Glo using standard methods and comparison to mock-treated reference wells.

TABLE 9
Exemplary CDH17 x CD3 Bispecific T cell antibodies

| | SEQ ID | |
|---|---|---|
| Construct Name | NO: | Sequence |
| TCE_CDH17_CD3_1 + 1 | 102 | QVQLQESGGGLVQPGGSTRLSCVSSRTFSY |
| | | YDMGWFRQAPGKEREFVALLSWRGNNAE |
| | | YSDSVMGRFTVSRGNTQNSVNLQMNNLKP |
| | | EDTGIYYCAVTYGGARSVRSWGQGTQVTV |
| | | SSGGGGSGGGGSDIKLQQSGAELARPGASV |
| | | KMSCKTSGYTFTRYTMHWVKQRPGQGLE |
| | | WIGYINPSRGYTNYNQKFKDKATLTTDKSS |
| | | STAYMQLSSLTSEDSAVYYCARYYDDHYC |
| | | LDYWGQGTTLTVSSVEGGSGGSGGSGGSG |
| | | GVDDIQLTQSPAIMSASPGEKVTMTCRASSS |
| | | VSYMNWYQQKSGTSPKRWIYDTSKVASGV |
| | | PYRFSGSGSGTSYSLTISSMEAEDAATYYCQ |
| | | QWSSNPLTFGAGTKLELK |
| TCE_CDH17_CD3_2 + 1_ | 103 | QVQLQESGGGLVQPGGSTRLSCVSSRTFSY |
| VH | | YDMGWFRQAPGKEREFVALLSWRGNNAE |
| | | YSDSVMGRFTVSRGNTQNSVNLQMNNLKP |
| | | EDTGIYYCAVTYGGARSVRSWGQGTQVTV |
| | | SSGGGGSGGGGSDIKLQQSGAELARPGASV |
| | | KMSCKTSGYTFTRYTMHWVKQRPGQGLE |
| | | WIGYINPSRGYTNYNQKFKDKATLTTDKSS |
| | | STAYMQLSSLTSEDSAVYYCARYYDDHYC |
| | | LDYWGQGTTLTVSSASTKGPSVFPLAPSSKS |
| | | TSGGTAALGCLVKDYFPEPVTVSWNSGALT |
| | | SGVHTFPAVLQSSGLYSLSSVVTVPSSSLGT |
| | | QTYICNVNHKPSNTKVDKRVEPKSCDKTHT |
| | | CPPCPAPEAAGGPSVFLFPPKPKDTLMISRTP |
| | | EVTCVVVDVSHEDPEVKFNWYVDGVEVHN |
| | | AKTKPREEQYNSTYRVVSVLTVLHQDWLN |
| | | GKEYKCKVSNKALGAPIEKTISKAKGQPRE |
| | | PQVCTLPPSREEMTKNQVSLSCAVKGFYPS |
| | | DIAVEWESNGQPENNYKTTPPVLDSDGSFF |
| | | LVSKLTVDKSRWQQGNVFSCSVMHEALHN |
| | | RFTQKSLSLSPGK |
| TCE_CDH17_CD3_2 + 1_ | 104 | DIQLTQSPAIMSASPGEKVTMTCRASSSVSY |
| VL | | MNWYQQKSGTSPKRWIYDTSKVASGVPYR |
| | | FSGSGSGTSYSLTISSMEAEDAATYYCQQW |
| | | SSNPLTFGAGTKLELKRTVAAPSVFIFPPSDE |
| | | QLKSGTASVVCLLNNFYPREAKVQWKVDN |
| | | ALQSGNSQESVTEQDSKDSTYSLSSTLTLSK |
| | | ADYEKHKVYACEVTHQGLSSPVTKSFNRGE |
| | | C |
| TCE_CDH17_CD3_2 + 2_ | 105 | QVQLQESGGGLVQPGGSTRLSCVSSRTFSY |
| VH | | YDMGWFRQAPGKEREFVALLSWRGNNAE |
| | | YSDSVMGRFTVSRGNTQNSVNLQMNNLKP |
| | | EDTGIYYCAVTYGGARSVRSWGQGTQVTV |
| | | SSGGGGSGGGGSDIKLQQSGAELARPGASV |
| | | KMSCKTSGYTFTRYTMHWVKQRPGQGLE |
| | | WIGYINPSRGYTNYNQKFKDKATLTTDKSS |
| | | STAYMQLSSLTSEDSAVYYCARYYDDHYC |
| | | LDYWGQGTTLTVSSASTKGPSVFPLAPSSKS |
| | | TSGGTAALGCLVKDYFPEPVTVSWNSGALT |
| | | SGVHTFPAVLQSSGLYSLSSVVTVPSSSLGT |
| | | QTYICNVNHKPSNTKVDKRVEPKSCDKTHT |
| | | CPPCPAPEAAGGPSVFLFPPKPKDTLMISRTP |
| | | EVTCVVVDVSHEDPEVKFNWYVDGVEVHN |
| | | AKTKPREEQYNSTYRVVSVLTVLHQDWLN |
| | | GKEYKCKVSNKALGAPIEKTISKAKGQPRE |
| | | PQVYTLPPSREEMTKNQVSLTCLVKGFYPS |
| | | DIAVEWESNGQPENNYKTTPPVLDSDGSFF |
| | | LYSKLTVDKSRWQQGNVFSCSVMHEALHN |
| | | HYTQKSLSLSPGK |
| TCE_CDH17_CD3_2 + 2_ | 106 | DIQLTQSPAIMSASPGEKVTMTCRASSSVSY |
| VL | | MNWYQQKSGTSPKRWIYDTSKVASGVPYR |
| | | FSGSGSGTSYSLTISSMEAEDAATYYCQQW |
| | | SSNPLTFGAGTKLELKRTVAAPSVFIFPPSDE |
| | | QLKSGTASVVCLLNNFYPREAKVQWKVDN |
| | | ALQSGNSQESVTEQDSKDSTYSLSSTLTLSK |
| | | ADYEKHKVYACEVTHQGLSSPVTKSFNRGE |
| | | C |

### Example 2. Expression of CDH17 VHH-Fc Fusion Drug Conjugates and Validation of Activity

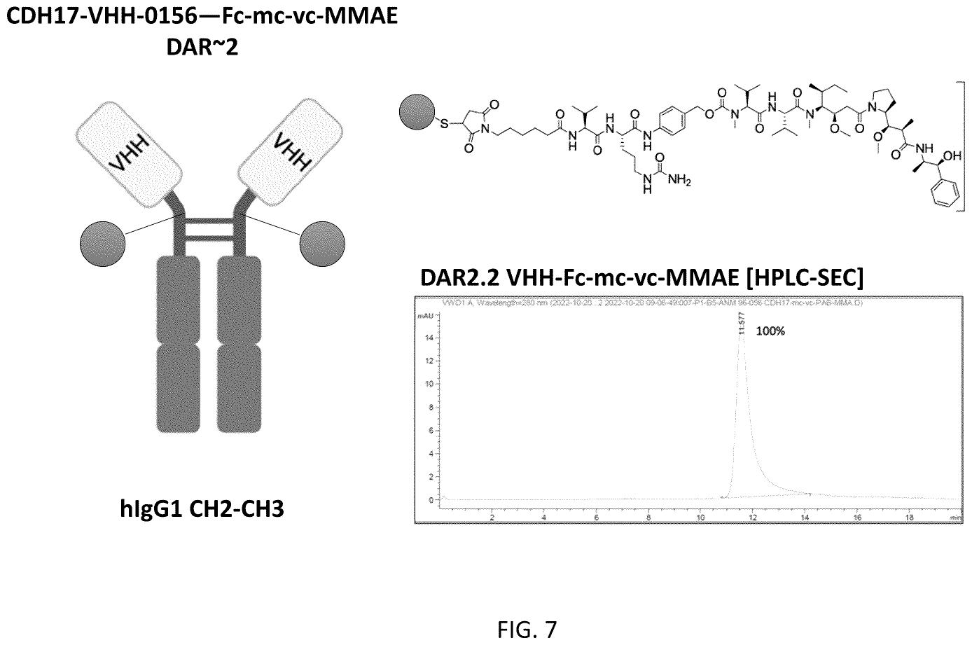

Anti-CDH17 VHH-Fc fusion protein drug bioconjugates were prepared using standard methods involving gentle reduction of interchain thiols. Briefly, the VHH-Fc proteins were incubated with various molar equivalents of TCEP (e.g. 2×, 5×, 10×) at 30° C. for 2 hr and subsequently mixed with various molar equivalents of commercial mc-vc-PAB-MMAE at 30° C. Free thiols were quenched, and free linker-payload was removed by dialysis. Purity was assessed by SEC-HPLC, RP-HPLC, and drug-antibody ratio (DAR) was assessed by HIC (FIG. 7).

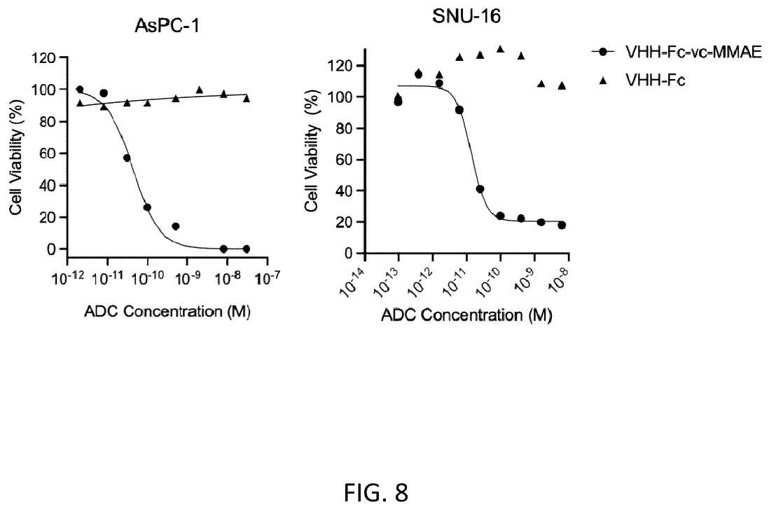

Cytotoxicity of anti-CDH17 VHH-Fc fusion protein drug bioconjugates to CDH17+ cells was determined by an ADC cytotoxicity assay (FIG. 8). To measure the cytotoxicity of VHH-Fc ADCs against CDH17+ cell lines, cell lines were first cultured using standard protocols. 1000 cells were seeded into each well of a standard assay well plate along with serial dilutions of ADC or unconjugated VHH-Fc fusion protein. Plates were incubated at 37° C. for 96 hours, and viable cells were quantitated by Cell Titer-Glo using manufacturer protocols.

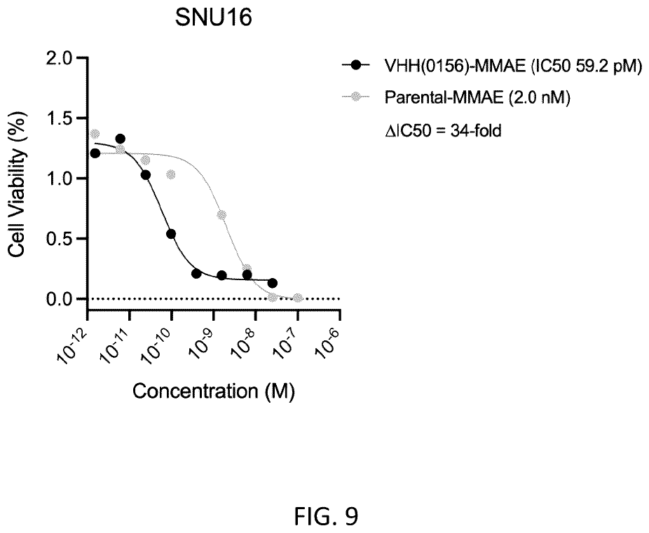

Using the same method, cytotoxicity of CDH17-VHH-0156-Fc and parental VHH-0001-Fc fusion protein mc-vc-PAB-MMAE conjugates (DAR2) in SNU16 cells was assessed (FIG. 9). The IC₅₀ of VHH-0156-MMAE conjugates to CDH17 was 34-fold lesser than the IC₅₀ of the parental-MMAE conjugates. This data demonstrates functional superiority (improved binding affinity and improved cytotoxicity) of VHH-0156-MMAE conjugates compared to the parental-MMAE conjugates.

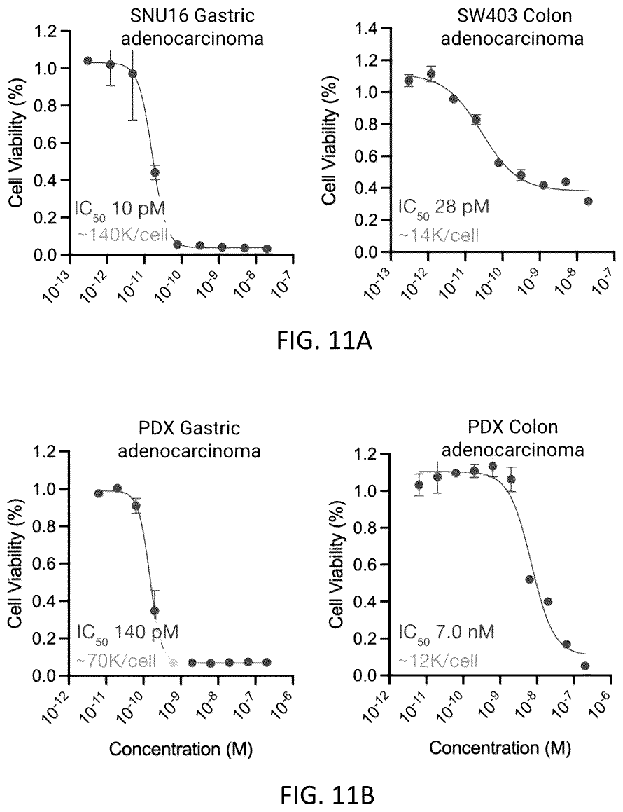

Cytotoxicity of BHB 156-vedotin (DAR4) and isotype control VHH-Fc-vedotin (DAR4) in SNU16 and SW403 cells (FIG. 11A) as well as patient derived xenograft (PDX) gastric adenocarcinoma tissue and PDX colon adenocarcinoma tissue (FIG. 11B) was assessed by an ADC cytotoxicity assay. To assess in vitro cytotoxicity of therapeutic candidate ADCs against representative cell lines, 3000 cells were seeded per well into a 96 well plate overnight. On the day of the experiment, serial dilutions of CDH17-VHH-0156-Fc and parental VHH-0001-Fc fusion protein mc-vc-PAB-MMAE conjugates (DAR˜4) were performed in cell growth medium. Serial dilutions were added to seeded cells, and cell viability was measured after 96 hours of drug exposure by addition of equal volumes of CellTiter-Glo reagent and plate reading (SpectraMax, Molecular Devices). Cell viability measurements were normalized to a mock-treated condition and plotted in Prism (GraphPad). IC₅₀ estimates are shown with a four parameter sigmoid fit model. Negative control/target-independent cell killing was measured by inclusion of 1 μM unconjugated BHB156 in all serial dilutions to saturate available CDH17 or inclusion of an isotype control VHH-Fc conjugated to vedotin with DAR 4.0. In addition, receptor density/Antibody Binding Capacity (ABC) was measured. Briefly, anti-CDH17 antibody BHB 156 were labeled with AF647 using standard NHS-ester chemistry (Invitrogen). Excess labeled antibody or isotype control was added to cells and cells were analyzed by flow cytometry (Agilent NovoCyte Advanteon). MFI was related to total antibody binding capacity by standard curve interpolation using MESF and QSC beads (Bangs Lab) according to the manufacturer's protocol. A range of observed sensitivity of culture-adapted (FIG. 11A) and primary patient derived xenograft (PDX) cancer cell tissues (FIG. 11B) to BHB156-vedotin (DAR4) in vitro was observed that correlates with CDH17 receptor density on the cell surface. Cell lines with less than 10,000 receptors per cell surface are minimally sensitive to the active drug compound. In all cases, an isotype control VHH-Fc conjugated to vedotin is minimally active.

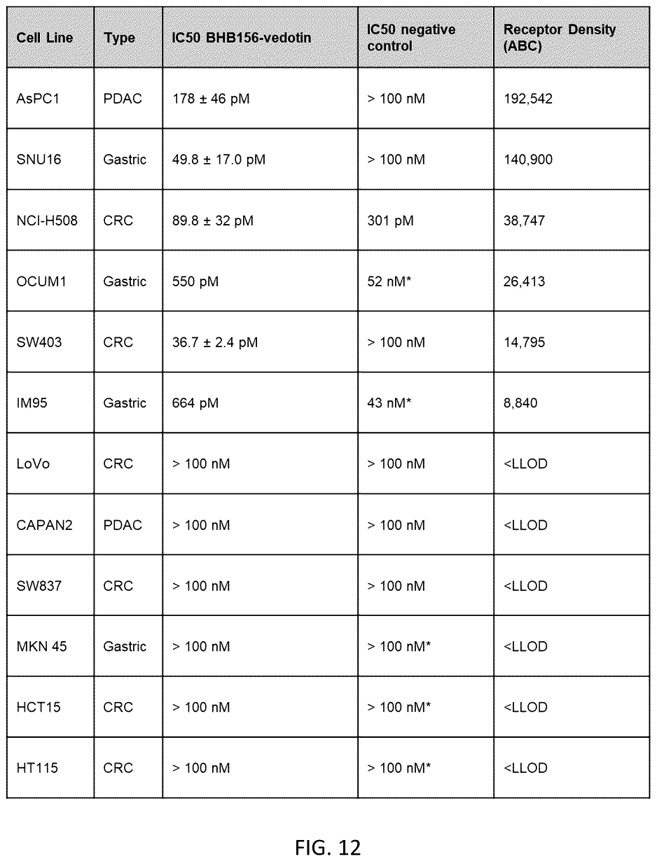

Using the same method, cytotoxicity BHB156-vedotin (DAR4) and isotype control VHH-Fc-vedotin (DAR4) in a number of cell lines representing a number of cancer types (FIG. 12) was assessed. AsPC1, SNU16, NCI-H508, OCUM1, SW403, IM95, LoVo, CAPAN2, SW837, MKN 45, HCT15, and HT115 cell lines were tested. The cell lines represented PDAC, Gastric, and CRC cancer types. IC₅₀ estimates were derived from serial dilutions of BHB156-vedotin (DAR4) or isotype control vedotin ADCs (DAR4) in vitro alongside receptor density measurements as described above. In some cases, we also performed negative control studies with free unconjugated VHH-Fc in ≥10-fold molar excess to block CDH17-dependent ADC uptake as denoted with an asterisk.

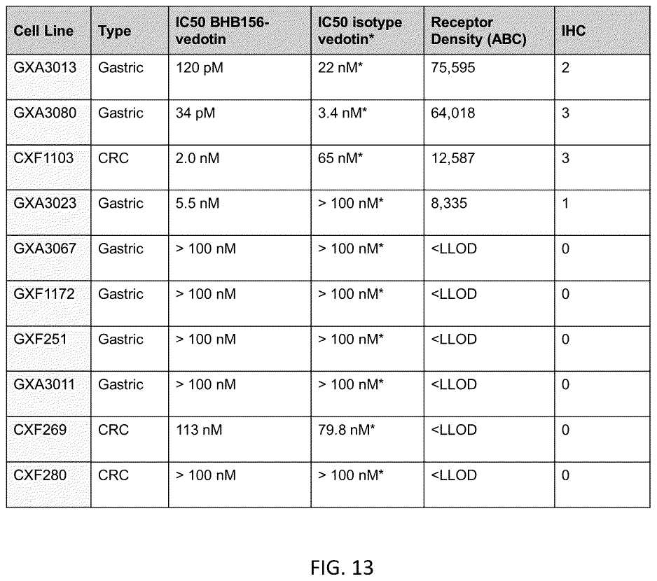

Using the same method, cytotoxicity BHB156-vedotin (DAR4) and isotype control VHH-Fc-vedotin (DAR4) in a number of cell lines representing a number of cancer types was assessed alongside receptor density measurements (ABC) and IHC score (FIG. 13). GXA3013, GXA3080, CXF1103, GXA3023, GXA3067, GXF1172, GXF251, GXA3011, CXF269, AND CXF280 cell lines were tested. The cell lines represented Gastric and CRC cancer types. IC₅₀ estimates were derived from serial dilutions of BHB 156-vedotin (DAR4) or isotype control vedotin ADCs in vitro alongside receptor density measurements as described above. Negative control sensitivity studies were performed with BHB 156-vedotin (DAR4) in the presence of >10-fold molar excess of free unconjugated VHH-Fc to block CDH17-dependent ADC uptake, as denoted with an asterisk.

TABLE 10
Exemplary CDH17 VHH-Fc fusion drug conjugates

| Construct Name | SEQ ID NO: | Sequence |
|---|---|---|
| CDH17_vhh_0001- | 107 | QVQLQESGGGLVQPGGSTRLSCVSSRTFSYYDMG |
| Fc | | WFRQAPGKEREFVALLSWNGENAEYSDSVMGRF |
| | | TVSRGNTQNSVNLQMNNLKPEDTGIYYCAVTHG |
| | | GARSVRSWGQGTQVTVSSGGGGSEPKSSDKTHT |
| | | CPPCPAPELLGGPSVFLFPPKPKDTLMISRTPEVTC |
| | | VVVDVSHEDPEVKFNWYVDGVEVHNAKTKPREE |
| | | QYNSTYRVVSVLTVLHQDWLNGKEYKCKVSNK |
| | | ALPAPIEKTISKAKGQPREPQVYTLPPSRDELTKN |
| | | QVSLTCLVKGFYPSDIAVEWESNGQPENNYKTTP |
| | | PVLDSDGSFFLYSKLTVDKSRWQQGNVFSCSVM |
| | | HEALHNHYTQKSLSLSPGK |
| CDH17_vhh_0156- | 108 | QVQLQESGGGLVQPGGSTRLSCVSSRTFSYYDMG |
| Fc | | WFRQAPGKEREFVALLSWRGNNAEYSDSVMGRF |
| | | TVSRGNTQNSVNLQMNNLKPEDTGIYYCAVTYG |
| | | GARSVRSWGQGTQVTVSSGGGGSEPKSSDKTHT |
| | | CPPCPAPELLGGPSVFLFPPKPKDTLMISRTPEVTC |
| | | VVVDVSHEDPEVKFNWYVDGVEVHNAKTKPREE |
| | | QYNSTYRVVSVLTVLHQDWLNGKEYKCKVSNK |
| | | ALPAPIEKTISKAKGQPREPQVYTLPPSRDELTKN |
| | | QVSLTCLVKGFYPSDIAVEWESNGQPENNYKTTP |
| | | PVLDSDGSFFLYSKLTVDKSRWQQGNVFSCSVM |
| | | HEALHNHYTQKSLSLSPGK |

### Example 3. CDH17 Expression in Primary Tumor Biopsy Core Samples

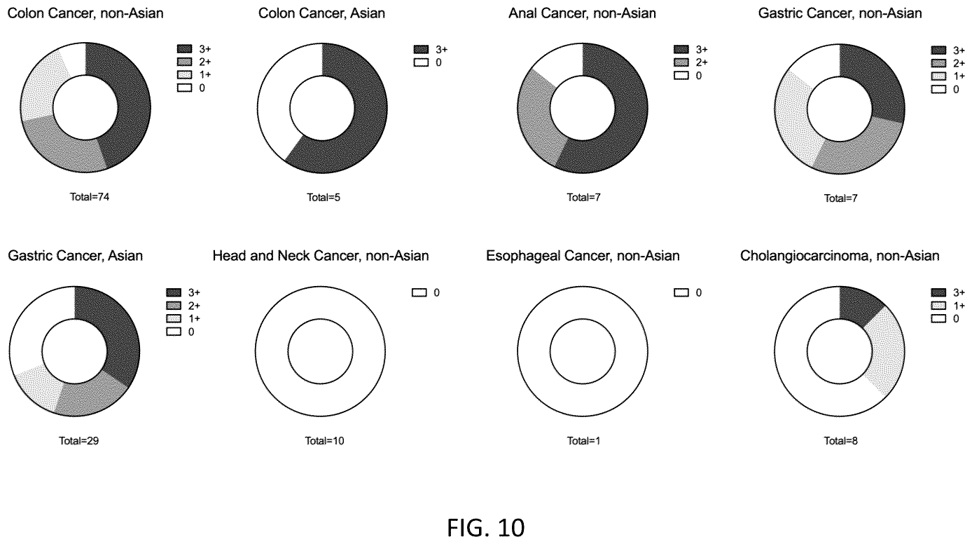

CDH17 expression in primary tumor biopsy core samples was assessed by immunohistochemistry (IHC) (FIG. 10). Excised patient derived xenograft (PDX) tumor tissues were fixed with formalin for 48 hours then transferred to 70% ethanol. Formalin-Fixed Paraffin-Embedded tissue blocks were generated by Histo-Tec Laboratory Inc. (Hayward, CA). Five μm unstained PDX, or microarray tissue sections were de-paraffinized in xylene and sequentially rehydrated with 100% ethanol, 96% ethanol, and deionized water. Antigen retrieval was performed in citrate buffer with 0.02% Tween 20 (pH=6.0) for 20 minutes at 95° C. Endogenous peroxidase activity was quenched using 3% hydrogen peroxidase and sections were blocked with 5% normal goat serum at 4° C. for 4 hours (Cell Signaling Technology®, Danvers, MA). Rabbit anti-hCDH17 antibody (Abcam Inc, Cambridge, MA) or an isotype control was used at 1:100 dilution in PBST and incubated overnight at 4° C. Using manufacturer's instructions, sections were incubated with SignalStain® Boost detection for 30 minutes at room temperature. Following sections were incubated with SignalStain® DAB Chromogen for 8 minutes (both reagents from Cell Signaling Technology®). Sections were counterstained with hematoxylin and placed in mounting medium (Cell Signaling Technology®). Finally, whole slide images were captured at 20× using the NanoZoomer 2.0-RS slide scanner for further analysis. IHC was scored from 0 to 3+ where 0 to 1+ is considered CDH17 negative, 2+ is considered borderline, and 3+ is considered CDH17 positive. The circle charts show the proportion of IHC scores for a particular cancer type. The samples were further divided into samples derived from non-Asian subjects or Asian subjects. The cancer types represented are colon cancer, anal cancer, gastric cancer, head and neck cancer, esophogeal cancer, and cholangiocarcinoma. Colon and anal cancers demonstrated the highest and most homogeneous levels of CDH17 expression. Approximately 25-40% of gastric cancers expressed high levels of CDH17, whereas cholangiocarcinomas did not widely express CDH17.

### Example 4. Plasma Elimination and Biodistribution of a Single Dose of I-125 Radiolabeled BHB156

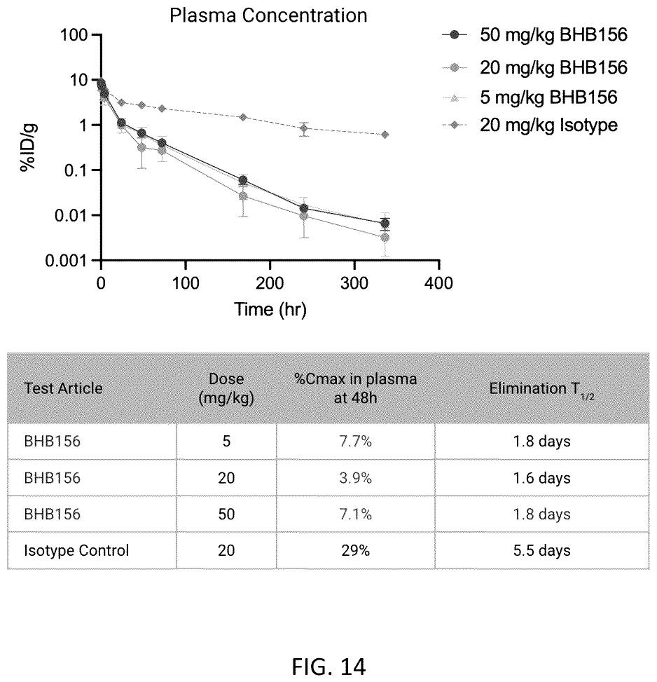

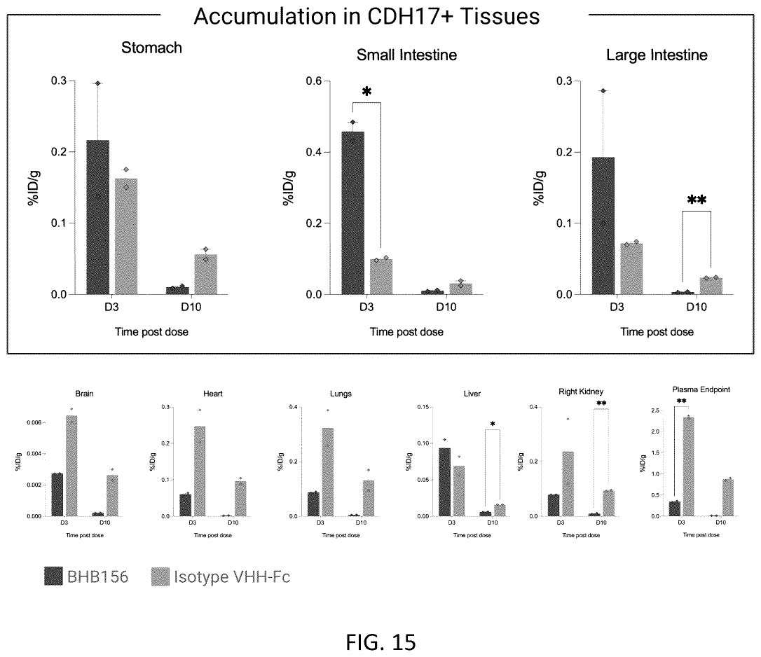

Plasma elimination of a single dose of I-125 radiolabeled BHB156 or anti-SARS-CoCV-2 isotype control VHH-Fc fusion protein was assessed in Sprague-Dawley rats at various concentrations and time points (FIG. 14). Iodine 125 labelling was performed using standard methods. CD® Sprague Dawley female rats (n=3 per group) received a single dose of 125I-BHB156 or 125I-BHB104. BHB156 was dosed at 1, 5, 20, or 50 mg/kg and BHB 104 was dosed at 20 mg/kg. In-life plasma samples were collected 15 minutes, 1 hour, 4 hours, 24 hours, 48 hours, 72 hours, 7 days, 10 days, 14 days post dose and urine samples were collected at 1-4 hours, 24 hours, 48 hours, and 72 hours post dose. Radioactivity of collected samples were measured by gamma counting. Counts were analyzed in Prism (GraphPad). Radiolabeled BHB156 was cleared from the plasma more rapidly than the isotype control VHH-Fc, demonstrating a half-life of about 2 days versus 5.5-6 days for the isotype. Greater than 90% of the injected BHB156 dose was distributed or metabolized from the plasma compartment within 48 hours of administration. Remarkably, there was no clear difference in clearance between 5, 20, and 50 mg/kg BHB156.

Biodistribution of a single dose of I-125 radiolabeled BHB156 in Sprague-Dawley rats was assessed 3 or 10 days after administration. Iodine 125 labelling was performed using standard methods. CD® Sprague Dawley female rats were dosed at 20 mg/kg with each compound for sacrifice at day 3 or day 10 for end-organ biodistribution analysis. Whole body perfusion was performed with cold saline prior to necropsy. Radioactivity of collected samples were measured by gamma counting. The stomach, large intestine, and small intestine samples were not rinsed prior to gamma counting. Counts were analyzed in Prism (GraphPad). Consistent with known CDH17 expression in the distal GI tract, BHB 156, cross-reactive to the rodent CDH17 orthologue, significantly accumulates in the small and large intestine after administration. Lower accumulation is seen in the brain, heart, lungs, and kidney versus the isotype control VHH-Fc. Similar, low accumulation is seen in the liver versus the isotype.

### Example 5. Potency of BHB156-Vedotin in Clearing Tumor Biomass in Cell Line Derived Xenograft (CDX) Tumor-Bearing Mice

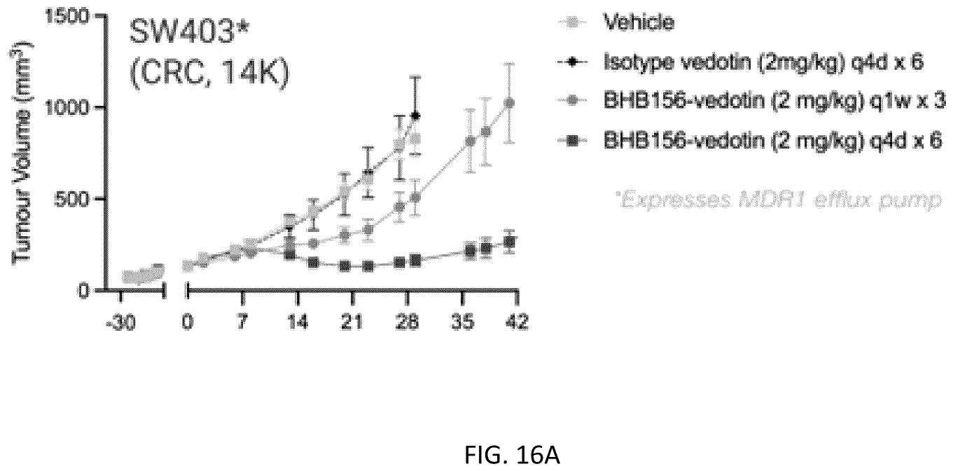

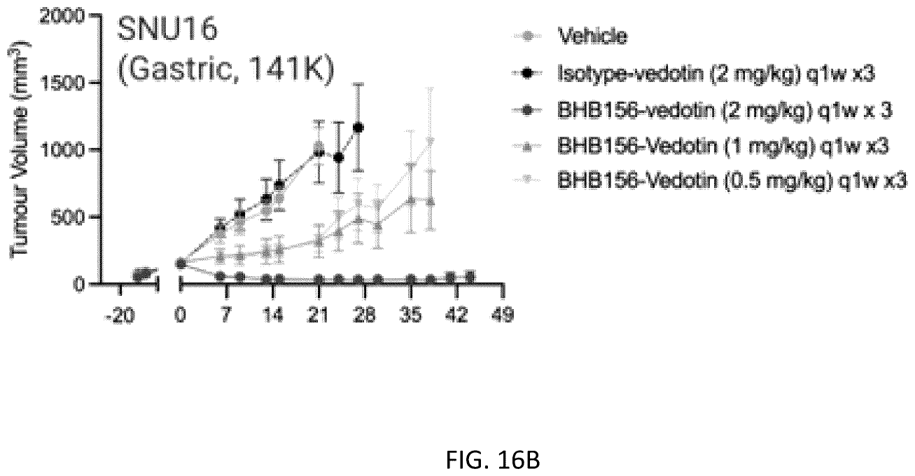

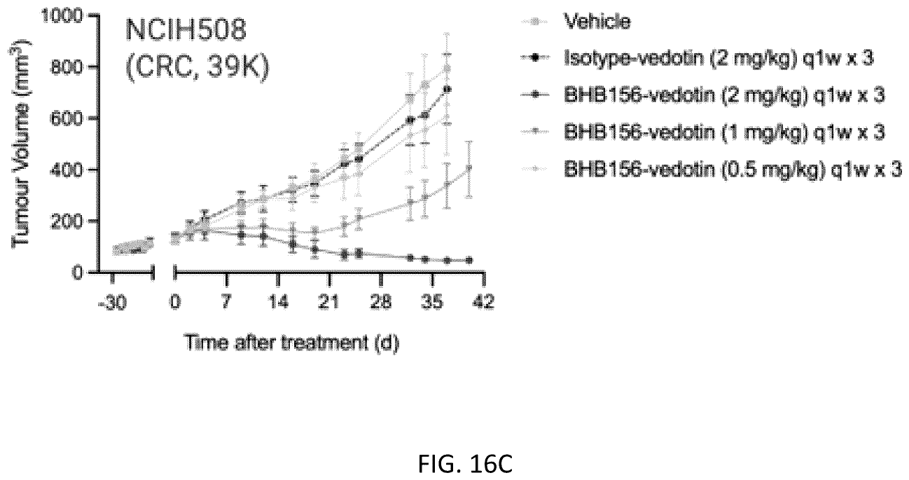

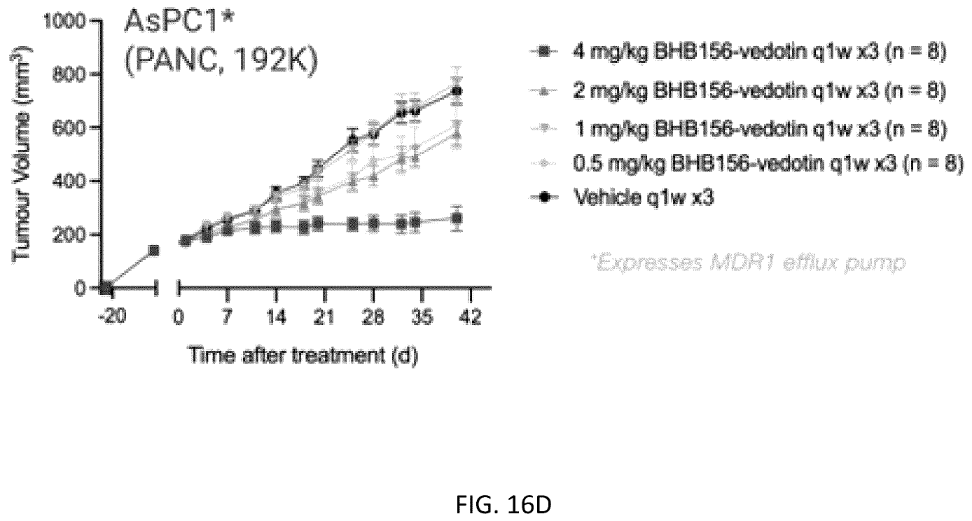

Potency of BHB156-vedotin (DAR4) versus isotype vedotin in clearing tumor biomass in CDX tumor-bearing mice was assessed (FIG. 16A-16D). Animals were maintained under standard housing and IACUC-approved protocols. Cell lines were obtained from ATCC (Manassas, VA) and cultured according to standard protocols. 5 million cells per animal were implanted in the right flank of nude mice (JAX Strain 002019) in matrigel. Once tumor volumes reached at least 125 mm³, animals were randomized for treatment into groups of at least 5 animals and treated with 0.5, 1, 2, or 4 mg/kg BHB 156-vedotin (DAR4) or vehicle or 2 mg/kg Isotype-vedotin. All tumors were measured out to 42 days after randomization and treatment with electronic calipers. Data were analyzed and plotted in Prism (GraphPad). In multiple model studies with differing cell-surface CDH17 levels, BHB156-vedotin (DAR4) dosing leads to sustained antitumor responses, whereas isotype vedotin ADCs are minimally effective compared to vehicle-treated animals. The best observed response in the AsPC1 model is stable disease, which correlates with the expression of high-capacity drug efflux pumps that extrude MMAE in vitro and in vivo. Inset parenthetical denotes primary cancer type and receptor density on the cell line from in vitro measurements prior to implantation.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An anti-CDH17 antibody or antigen-binding fragment thereof comprising:
(i) (a) a HCDR1 having the amino acid sequence set out in SEQ ID NO: 41, (b) a HCDR2 having the amino acid sequence set out in any one of SEQ ID NOs: 43-60, and (c) a HCDR3 having at least 90% sequence identity to the amino acid sequence set out in any one of SEQ ID NOs: 61-75; or
(ii) (a) a HCDR1 having the amino acid sequence set out in SEQ ID NO: 41, (b) a HCDR2 having at least 90% sequence identity to the amino acid sequence set out in any one of SEQ ID NOs: 42-60, and (c) a HCDR3 having the amino acid sequence set out in any one of SEQ ID NOs: 62-75.

2.-3. (canceled)

4. An anti-CDH17 antibody or antigen-binding fragment thereof comprising (a) a HCDR1 having an amino acid sequence YDMG (SEQ ID NO: 41); (b) a HCDR2 having an amino acid sequence LLSWRGX₂NAEYX₃DX₄VX₅G (SEQ ID NO: 79), wherein X₂ is E or N, X₃ is S or R, X₄ is S, D, or F, and X₅ is M or H; and (c) a HCDR3 having an amino acid sequence TX₆GX₇AX₈SVRX₉ (SEQ ID NO: 80), wherein X₆ is H or Y, X₇ is G, T, V, K or N, X₈ is R or K, and X₉ is S or L.

5. The anti-CDH17 antibody or antigen-binding fragment thereof of claim 4, wherein X₂ is N, X₃ is S, X₄ is S, and X₅ is M, X₆ is Y, X₇ is G, X₈ is R, and X₉ is S.

6. The anti-CDH17 antibody or antigen-binding fragment thereof of claim 1, wherein the anti-CDH17 antibody or antigen-binding fragment thereof comprises an amino acid sequence having at least 90% sequence identity to the amino acid sequence set out in any one of SEQ ID NO: 2-40 or 109.

7. The anti-CDH17 antibody or antigen-binding fragment thereof of claim 1, wherein the anti-CDH17 antibody or antigen-binding fragment thereof comprises a HCDR1 as set out in SEQ ID NO: 41, a HCDR2 as set out in SEQ ID NO: 47, and/or a HCDR3 as set out in SEQ ID NO: 72.

8. (canceled)

9. The anti-CDH17 antibody or antigen-binding fragment thereof of claim 1, wherein the anti-CDH17 antibody or antigen-binding fragment thereof has an increased binding affinity to CDH17 compared to a binding affinity of an amino acid sequence as set out in SEQ ID NO: 1 to CDH17, and wherein the increased binding affinity of the anti-CDH17 antibody or antigen-binding fragment thereof to CDH17 is at least 3-fold, at least 5-fold, at least 10-fold, or at least 15-fold greater than the binding affinity of an amino acid sequence as set out in SEQ ID NO: 1 to CDH17.

10. (canceled)

11. The anti-CDH17 antibody or antigen-binding fragment thereof of claim 1, wherein the anti-CDH17 antibody or antigen-binding fragment thereof has a KD for binding to CDH17 of less than 1.0×10⁻⁸ M, less than 5.0×10⁻⁹ M, or less than 3.0×10⁻⁹ M.

12.-15. (canceled)

16. The anti-CDH17 antibody or antigen-binding fragment thereof of claim 1, wherein the anti-CDH17 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof.

17.-19. (canceled)

20. A conjugate comprising Formula (I):
Ab-(L-D)n Formula (I)
wherein Ab comprises an anti-CDH17 antibody or antigen-binding fragment thereof, wherein anti-CDH17 antibody or antigen-binding fragment thereof comprising (a) a HCDR1 having the amino acid sequence set out in SEQ ID NO: 41, (b) a HCDR2 having at least 90% sequence identity to the amino acid sequence set out in any one of SEQ ID NOs: 43-60, and (c) a HCDR3 having at least 90% sequence identity to the amino acid sequence set out in any one of SEQ ID NOs: 61-75;
L is a linker;
D is a cytotoxic agent; and
n is a drug antibody ratio, wherein the drug antibody ratio is from about 1 to about 8.

21. The conjugate of claim 20, wherein the anti-CDH17 antibody or antigen-binding fragment thereof comprises:
(i) (a) a HCDR1 having an amino acid sequence as set out in SEQ ID NO: 41, (b) a HCDR2 having an amino acid sequence as set out in SEQ ID NO: 47, and (c) a HCDR3 having an amino acid sequence as set out in SEQ ID NO: 72; or
(ii) an amino acid sequence having at least 90% sequence identity to the amino acid sequence set out in any one of SEQ ID NO: 2-40 or 109.

22.-23. (canceled)

24. The conjugate of claim 20, wherein the anti-CDH17 antibody or antigen-binding fragment thereof further comprises a Fc domain.

25. (canceled)

26. The conjugate of claim 20, wherein the cytotoxic agent comprises a toxin targeting ribosomes, a toxin targeting elongation factors, a toxin targeting tubulin, a toxin targeting DNA, a toxin targeting RNA, emtansine, pasudotox, maytansinoid derivative DM1, maytansinoid derivative DM4, a pyrrolobenzodiazepine (PBD) dimer, a benzodiazepine, a CC-1065 analogue, paclitaxel, docetaxel, cisplatin, cyclophosphamide, etoposide, 5-fluorouracyl (5-FU), mitoxantrone, an indolinobenzodiazepine, AZ13599185, a cryptophycin, rhizoxin, methotrexate, an anthracycline, a camptothecin analogue, DX-8951f, exatecan mesylate, a duocarmycin derivative, an amanitin, a-amanitin, a spliceostatin, a thailanstatin, ozogamicin, Amberstatin269, soravtansine, dolastatin 10, auristatin E, auristatin EB (AEB), auristatin EFP (AEFP), Monomethyl Auristatin D (MMAD), monomethyl dolastatin 10, Monomethyl Auristatin F (MMAF, mafodotin), N-methylvaline-valine-dolaisoleuine-dolaproine-phenylalanine, Monomethyl Auristatin E (MMAE, vedotin), N-methylvaline-valine-dolaisoleuine-dolaproine-norephedrine, deruxtecan, tesirine, mertansine, ravtansine, duocarmycin, calicheamicin, N-acetyl-γ-calicheamicin, maytansinoid, pyrrolobenzodiazepine (PBD), doxorubicin, anthracyclines, camptothecin derivatives, taxanes, hedgehog inhibitors, nitrogen mustards, and histone deacetylase inhibitors, PE38, SN-38, ENPP3, tubulysin, exatecan, STING agonist, TLR agonist, alpha-Amanitin, SGD-1882, CC-1065 or 5-benzoylvaleric acid-AE ester (AEVB).

27. The conjugate of claim 26, wherein the cytotoxic agent is MMAE.

28. The conjugate of claim 20, wherein the linker comprises a bond, a cleavable linker, a non-cleavable linker, an attachment group, a protease-cleavable linker, and/or a spacer.

29. (canceled)

30. The conjugate of claim 28, wherein the attachment group comprises maleimide and/or caproic acid.

31. The conjugate of claim 28, wherein the protease-cleavable linker is a cathepsin-cleavable linker.

32.-33. (canceled)

34. The conjugate of claim 28, wherein the spacer is PABC.

35. The conjugate of claim 20, wherein n is about 4.

36.-40. (canceled)

41. A pharmaceutical composition for treating cancer, comprising the anti-CDH17 antibody or antigen-binding fragment thereof of claim 1 and a pharmaceutically acceptable carrier or excipient.

42.-44. (canceled)

45. A method for treating cancer in a subject in need thereof comprising administering to the subject a therapeutically effective amount of the anti-CDH17 antibody or antigen-binding fragment thereof.

46.-50. (canceled)


SEQUENCE LISTING (114 sequences)

SEQ ID NO: 1 - protein, 118 aa, synthetic construct
QVQLQESGGGLVQPGGSTRLSCVSSRTFSYYDMGWFRQAPGKEREFVALLSWNGENAEYSDSVMGRFTVSRGNTQNSVNLQMNNLKPEDTGIYYCAVTHGGARSVRSWGQGTQVTVSS

SEQ ID NO: 2 - protein, 118 aa, synthetic construct
QVQLQESGGGLVQPGGSTRLSCVSSRTFSYYDMGWFRQAPGKEREFVALLSWRGNNAEYSDDVMGRFTVSRGNTQNSVNLQMNNLKPEDTGIYYCAVTYGTARSVRLWGQGTQVTVSS

SEQ ID NO: 3 - protein, 118 aa, synthetic construct
QVQLQESGGGLVQPGGSTRLSCVSSRTFSYYDMGWFRQAPGKEREFVALLSWRGNNAEYRDSVMGRFTVSRGNTQNSVNLQMNNLKPEDTGIYYCAVTYGVAKSVRLWGQGTQVTVSS

SEQ ID NO: 4 - protein, 118 aa, synthetic construct
QVQLQESGGGLVQPGGSTRLSCVSSRTFSYYDMGWFRQAPGKEREFVALLSWRGNNAEYSDDVMGRFTVSRGNTQNSVNLQMNNLKPEDTGIYYCAVTYGKARSVRLWGQGTQVTVSS

SEQ ID NO: 5 - protein, 118 aa, synthetic construct
QVQLQESGGGLVQPGGSTRLSCVSSRTFSYYDMGWFRQAPGKEREFVALLSWRGNNAEYSDFVMGRFTVSRGNTQNSVNLQMNNLKPEDTGIYYCAVTYGVARSVRLWGQGTQVTVSS

SEQ ID NO: 6 - protein, 118 aa, synthetic construct
QVQLQESGGGLVQPGGSTRLSCVSSRTFSYYDMGWFRQAPGKEREFVALLSWRGNNAEYSDSVHGRFTVSRGNTQNSVNLQMNNLKPEDTGIYYCAVTYGVARSVRLWGQGTQVTVSS

SEQ ID NO: 7 - protein, 118 aa, synthetic construct
QVQLQESGGGLVQPGGSTRLSCVSSRTFSYYDMGWFRQAPGKEREFVALLSWRGNNAEYSDSVMGRFVVSRGNTQNSVNLQMNNLKPEDTGIYYCAVTYGVAKSVRSWGQGTQVTVSS

SEQ ID NO: 8 - protein, 118 aa, synthetic construct
QVQLQESGGGLVQPGGSTRLSCVSSRTFSYYDMGWFRQAPGKEREFVALLSWRGNNAEYRDSVMGRFTVSRGNTQNSVNLQMNNLKPEDTGIYYCAVTYGGAKSVRLWGQGTQVTVSS

SEQ ID NO: 9 - protein, 118 aa, synthetic construct
QVQLQESGGGLVQPGGSTRLSCVSSRTFSYYDMGWFRQAPGKEREFVALLSWRGNNAEYRDSVMGRFTVSRGNTQNSVNLQMNNLKPEDTGIYYCAVTYGGARSVRLWGQGTQVTVSS

SEQ ID NO: 10 - protein, 118 aa, synthetic construct
QVQLQESGGGLVQPGGSTRLSCVSSRTFSYYDMGWFRQAPGKEREFVALLSWRGNNAEYSDDVMGRFTVSRGNTQNSVNLQMNNLKPEDTGIYYCAVTYGNARSVRLWGQGTQVTVSS

SEQ ID NO: 11 - protein, 118 aa, synthetic construct
QVQLQESGGGLVQPGGSTRLSCVSSRTFSYYDMGWFRQAPGKEREFVALLSWRGNNAEYSDDVMGRFTVSRGNTQNSVNLQMNNLKPEDTGIYYCAVTYGKARSVRSWGQGTQVTVSS

SEQ ID NO: 12 - protein, 118 aa, synthetic construct
QVQLQESGGGLVQPGGSTRLSCVSSRTFSYYDMGWFRQAPGKEREFVALLSWRGNNAEYSDSVMGRFVVSRGNTQNSVNLQMNNLKPEDTGIYYCAVTYGVARSVRSWGQGTQVTVSS

SEQ ID NO: 13 - protein, 118 aa, synthetic construct
QVQLQESGGGLVQPGGSTRLSCVSSITFSYYDMGWFRQAPGKEREFVALLSWRGENAEYSDSVMGRFTVSRGNTQNSVNLQMNNLKPEDTGIYYCAVTYGGARSVRSWGQGTQVTVSS

SEQ ID NO: 14 - protein, 118 aa, synthetic construct
QVQLQESGGGLVQPGGSTRLSCVSSRTFSYYDMGWFRQAPGKEREFVALLSWRGINAEYSDTVMGRFVVSRGNTQNSVNLQMNNLKPEDTGIYYCAVTYGVARSVRSWGQGTQVTVSS

SEQ ID NO: 15 - protein, 118 aa, synthetic construct
QVQLQESGGGLVQPGGSTRLSCVSSRTFSYYDMGWFRQAPGKEREFVALLSWRGNNAEYSDSVMGRFTVSRGNTQNSVNLQMNNLKPEDTGIYYCAVTYGGARSVRLWGQGTQVTVSS

SEQ ID NO: 16 - protein, 118 aa, synthetic construct
QVQLQESGGGLVQPGGSTRLSCVSSRTFSYYDMGWFRQAPGKEREFVALLSWRGNNAEYRDSVMGRFVVSRGNTQNSVNLQMNNLKPEDTGIYYCAVTYGVARSVRSWGQGTQVTVSS

SEQ ID NO: 17 - protein, 118 aa, synthetic construct
QVQLQESGGGLVQPGGSTRLSCVSSRTFSYYDMGWFRQYPGKEREFVALLSWRGNYAEYSDSVMGRFVVSRGNTQNSVNLQMNNLKPEDTGIYYCAVTYGGARSVRLWGQGTQVTVSS

SEQ ID NO: 18 - protein, 118 aa, synthetic construct
QVQLQESGGGLVQPGGSTRLSCVSSRTFSYYDMGWFRQYPGKEREFVALLSWRGNNAEYSDSVMGRFVVSRGNTQNSVNLQMNNLKPEDTGIYYCAVTLGVARSVRSWGQGTQVTVSS

SEQ ID NO: 19 - protein, 118 aa, synthetic construct
QVQLQESGGGLVQPGGSTRLSCVSSRTFSYYDMGWFRQAPGKEREFVALLSWRGNNAEYSDSVHGRFTVSRGNTQNSVNLQMNNLKPEDTGIYYCAVTYGGARSVRLWGQGTQVTVSS

SEQ ID NO: 20 - protein, 118 aa, synthetic construct
QVQLQESGGGLVQPGGSTRLSCVSSRTFSYYDMGWFRQAPGKEREFVALLSWRGNNAEYSDTVMGRFTVSRGNTQNSVNLQMNNLKPEDTGIYYCAVTYGGARSVRKWGQGTQVTVSS

SEQ ID NO: 21 - protein, 118 aa, synthetic construct
QVQLQESGGGLVQPGGSTRLSCVSSRTFSYYDMGWFRQAPGKEREFVALLSWRGNNSEYSDSVHGRFTVSRGNTQNSVNLQMNNLKPEDTGIYYCAVTYGGARSVRLWGQGTQVTVSS

SEQ ID NO: 22 - protein, 118 aa, synthetic construct
QVQLQESGGGLVQPGGSTRLSCVSSRTFSYYDMGWFRQAPGKEREFVALLSWRGNNAEYSDTVMGRFTVSRGNTQNSVNLQMNNLKPEDTGIYYCAVTYGGARSVRAWGQGTQVTVSS

SEQ ID NO: 23 - protein, 118 aa, synthetic construct
QVQLQESGGGLVQPGGSTRLSCVSSRTFSYYDMGWFRQAPGKEREFVALLSWRGNYAEYSDSVMGRFVVSRGNTQNSVNLQMNNLKPEDTGIYYCAVTYGGARSVRSWGQGTQVTVSS

SEQ ID NO: 24 - protein, 118 aa, synthetic construct
QVQLQESGGGLVQPGGSTRLSCVSSRTFSYYDMGWFRQAPGKEREFVALLSWRGNNAEYSDDVHGRFTVSRGNTQNSVNLQMNNLKPEDTGIYYCAVTYGGARSVRLWGQGTQVTVSS

SEQ ID NO: 25 - protein, 118 aa, synthetic construct
QVQLQESGGGLVQPGGSTRLSCVSSRTFSYYDMGWFRQAPGKEREFVALLSWRGNNAEYSDSVMGRFVVSRGNTQNSVNLQMNNLKPEDTGIYYCAVTYGGAKSVRSWGQGTQVTVSS

SEQ ID NO: 26 - protein, 118 aa, synthetic construct
QVQLQESGGGLVQPGGSTRLSCVSSRTFSYYDMGWFRQAPGKEREFVALLSWRGNNAEYSDDVMGRFTVSRGNTQNSVNLQMNNLKPEDTGIYYCAVTYGTARSVRSWGQGTQVTVSS

SEQ ID NO: 27 - protein, 118 aa, synthetic construct
QVQLQESGGGLVQPGGSTRLSCVSSRTFSYYDMGWFRQAPGKEREFVALLDWRGNYAEYSDSVMGRFTVSRGNTQNSVNLQMNNLKPEDTGIYYCAVTYGGARSVRSWGQGTQVTVSS

SEQ ID NO: 28 - protein, 118 aa, synthetic construct
QVQLQESGGGLVQPGGSTRLSCVSSRTFSYYDMGWFRQYPGKEREFVALLSWRGNNAEYSDSVMGRFTVSRGNTQNSVNLQMNNLKPEDTGIYYCAVTYGGARSVRSWGQGTQVTVSS

SEQ ID NO: 29 - protein, 118 aa, synthetic construct
QVQLQESGGGLVQPGGSTRLSCVSSRTFSYYDMGWFRQAPGKEREFVALLDWRGNNAEYSDSVMGRFTVSRGNTQNSVNLQMNNLKPEDTGIYYCAVTYGGARSVRSWGQGTQVTVSS

SEQ ID NO: 30 - protein, 118 aa, synthetic construct
QVQLQESGGGLVQPGGSTRLSCVSSRTFSYYDMGWFRQAPGKEREFVALLSWRGENAEYSDSVMGRFTVSRGNTQNSVNLQMNNLKPEDTGIYYCAVTYGGARSVRSWGQGTQVTVSS

SEQ ID NO: 31 - protein, 118 aa, synthetic construct
QVQLQESGGGLVQPGGSTRLSCVSSRTFSYYDMGWFRQAPGKEREFVALLSWRGERAEYSDSVMGRFTVSRGNTQNSVNLQMNNLKPEDTGIYYCAVTYGGARSVRSWGQGTQVTVSS

SEQ ID NO: 32 - protein, 118 aa, synthetic construct
QVQLQESGGGLVQPGGSTRLSCVSSRTFSYYDMGWFRQAPGKEREFVALLSWRGNNAEYSDFVMGRFTVSRGNTQNSVNLQMNNLKPEDTGIYYCAVTYGGARSVRLWGQGTQVTVSS

SEQ ID NO: 33 - protein, 118 aa, synthetic construct
QVQLQESGGGLVQPGGSTRLSCVSSRTFSYYDMGWFRQAPGKEREFVALLSWRGNNAEYRDSVMGRFTVSRGNTQNSVNLQMNNLKPEDTGIYYCAVTYGGARSVRSWGQGTQVTVSS

SEQ ID NO: 34 - protein, 118 aa, synthetic construct
QVQLQESGGGLVQPGGSTRLSCVSSRTFSYYDMGWFRQAPGKEREFVALLDWRGENAEYSDSVMGRFTVSRGNTQNSVNLQMNNLKPEDTGIYYCAVTYGGARSVRSWGQGTQVTVSS

SEQ ID NO: 35 - protein, 118 aa, synthetic construct
QVQLQESGGGLVQPGGSTRLSCVSSRTFSYYDMGWFRYAPGKEREFVALLSWRGNNAEYADSVMGRFTVSRGNTQNSVNLQMNNLKPEDTGIYYCAVTYGGARSVRSWGQGTQVTVSS

SEQ ID NO: 36 - protein, 118 aa, synthetic construct
QVQLQESGGGLVQPGGSTRLSCVSSRTFSYYDMGWFRQAPGKEREFVALLSWRGINAEYSDSVMGRFTVSRGNTQNSVNLQMNNLKPEDTGIYYCAVTYGGARSVRSWGQGTQVTVSS

SEQ ID NO: 37 - protein, 118 aa, synthetic construct
QVQLQESGGGLVQPGGSTRLSCVSSRTFSYYDMGWFRQAPGKEREFVALLSWRGNNAEYSDTVMGRFTVSRGNTQNSVNLQMNNLKPEDTGIYYCAVTYGGARSVRSWGQGTQVTVSS

SEQ ID NO: 38 - protein, 118 aa, synthetic construct
QVQLQESGGGLVQPGGSTRLSCVSSRTFSYYDMGWFRQYPGKEREFVALLSWRGNYAEYSDSVMGRFVVSRGNTQNSVNLQMNNLKPEDTGIYYCAVTYGGARSVRSWGQGTQVTVSS

SEQ ID NO: 39 - protein, 118 aa, synthetic construct
QVQLQESGGGLVQPGGSTRLSCVSSRTFSYYDMGWFRQAPGKEREFVALLSWRGNNAEYSESVMGRFTVSRGNTQNSVNLQMNNLKPEDTGIYYCAVTYGGARSVRSWGQGTQVTVSS

SEQ ID NO: 40 - protein, 118 aa, synthetic construct
QVQLQESGGGLVQPGGSTRLSCVSSRTFSYYDMGWFRQAPGKEREFVALLSWRGNNAEYSDDVMGRFTVSRGNTQNSVNLQMNNLKPEDTGIYYCAVTYGNARSVRSWGQGTQVTVSS

SEQ ID NO: 41 - protein, 4 aa, synthetic construct
YDMG

SEQ ID NO: 42 - protein, 17 aa, synthetic construct
LLSWNGENAEYSDSVMG

SEQ ID NO: 43 - protein, 17 aa, synthetic construct
LLSWRGNNAEYSDDVMG

SEQ ID NO: 44 - protein, 17 aa, synthetic construct
LLSWRGNNAEYRDSVMG

SEQ ID NO: 45 - protein, 17 aa, synthetic construct
LLSWRGNNAEYSDFVMG

SEQ ID NO: 46 - protein, 17 aa, synthetic construct
LLSWRGNNAEYSDSVHG

SEQ ID NO: 47 - protein, 17 aa, synthetic construct
LLSWRGNNAEYSDSVMG

SEQ ID NO: 48 - protein, 17 aa, synthetic construct
LLSWRGENAEYSDSVMG

SEQ ID NO: 49 - protein, 17 aa, synthetic construct
LLSWRGINAEYSDTVMG

SEQ ID NO: 50 - protein, 17 aa, synthetic construct
LLSWRGNYAEYSDSVMG

SEQ ID NO: 51 - protein, 17 aa, synthetic construct
LLSWRGNNAEYSDTVMG

SEQ ID NO: 52 - protein, 17 aa, synthetic construct
LLSWRGNNSEYSDSVHG

SEQ ID NO: 53 - protein, 17 aa, synthetic construct
LLSWRGNNAEYSDDVHG

SEQ ID NO: 54 - protein, 17 aa, synthetic construct
LLDWRGNYAEYSDSVMG

SEQ ID NO: 55 - protein, 17 aa, synthetic construct
LLDWRGNNAEYSDSVMG

SEQ ID NO: 56 - protein, 17 aa, synthetic construct
LLSWRGERAEYSDSVMG

SEQ ID NO: 57 - protein, 17 aa, synthetic construct
LLDWRGENAEYSDSVMG

SEQ ID NO: 58 - protein, 17 aa, synthetic construct
LLSWRGNNAEYADSVMG

SEQ ID NO: 59 - protein, 17 aa, synthetic construct
LLSWRGINAEYSDSVMG

SEQ ID NO: 60 - protein, 17 aa, synthetic construct
LLSWRGNNAEYSESVMG

SEQ ID NO: 61 - protein, 10 aa, synthetic construct
THGGARSVRS

SEQ ID NO: 62 - protein, 10 aa, synthetic construct
TYGTARSVRL

SEQ ID NO: 63 - protein, 10 aa, synthetic construct
TYGVAKSVRL

SEQ ID NO: 64 - protein, 10 aa, synthetic construct
TYGKARSVRL

SEQ ID NO: 65 - protein, 10 aa, synthetic construct
TYGVARSVRL

SEQ ID NO: 66 - protein, 10 aa, synthetic construct
TYGVAKSVRS

SEQ ID NO: 67 - protein, 10 aa, synthetic construct
TYGGAKSVRL

SEQ ID NO: 68 - protein, 10 aa, synthetic construct
TYGGARSVRL

SEQ ID NO: 69 - protein, 10 aa, synthetic construct
TYGNARSVRL

SEQ ID NO: 70 - protein, 10 aa, synthetic construct
TYGKARSVRS

SEQ ID NO: 71 - protein, 10 aa, synthetic construct
TYGVARSVRS

SEQ ID NO: 72 - protein, 10 aa, synthetic construct
TYGGARSVRS

SEQ ID NO: 73 - protein, 10 aa, synthetic construct
TLGVARSVRS

SEQ ID NO: 74 - protein, 10 aa, synthetic construct
TYGGARSVRK

SEQ ID NO: 75 - protein, 10 aa, synthetic construct
TYGGARSVRA

SEQ ID NO: 76 - protein, 10 aa, synthetic construct
TYGGAKSVRS

SEQ ID NO: 77 - protein, 10 aa, synthetic construct
TYGTARSVRS

SEQ ID NO: 78 - protein, 10 aa, synthetic construct
TYGNARSVRS

SEQ ID NO: 79 - protein, 17 aa, synthetic construct
  VARIANT 7: note = E or N
  VARIANT 12: note = S or R
  VARIANT 14: note = S, D, or F
  VARIANT 16: note = M or H
LLSWRGXNAEYXDXVXG

SEQ ID NO: 80 - protein, 10 aa, synthetic construct
  VARIANT 2: note = H or Y
  VARIANT 4: note = G, T, V, K or N
  VARIANT 6: note = R or K
  VARIANT 10: note = S or L
TXGXAXSVRX

SEQ ID NO: 81 - protein, 17 aa, synthetic construct
  VARIANT 5: note = N or R
  VARIANT 12: note = S or R
  VARIANT 14: note = S, D, or F
  VARIANT 16: note = M or H
LLSWXGNNAEYXDXVXG

SEQ ID NO: 82 - protein, 17 aa, synthetic construct
  VARIANT 5: note = N or R
  VARIANT 7: note = E or N
  VARIANT 14: note = S, D, or F
  VARIANT 16: note = M or H
LLSWXGXNAEYRDXVXG

SEQ ID NO: 83 - protein, 17 aa, synthetic construct
  VARIANT 5: note = N or R
  VARIANT 7: note = E or N
  VARIANT 12: note = S or R
  VARIANT 14: note = D or F
  VARIANT 16: note = M or H
LLSWXGXNAEYXDXVXG

SEQ ID NO: 84 - protein, 17 aa, synthetic construct
  VARIANT 5: note = N or R
  VARIANT 7: note = E or N
  VARIANT 12: note = S or R
  VARIANT 14: note = S, D, or F
LLSWXGXNAEYXDXVHG

SEQ ID NO: 85 - protein, 10 aa, synthetic construct
  VARIANT 4: note = G, T, V, K or N
  VARIANT 6: note = R or K
  VARIANT 10: note = S or L
TYGXAXSVRX

SEQ ID NO: 86 - protein, 10 aa, synthetic construct
  VARIANT 2: note = H or Y
  VARIANT 4: note = G, T, V, K or N
  VARIANT 10: note = S or L
TXGXAKSVRX

SEQ ID NO: 87 - protein, 10 aa, synthetic construct
  VARIANT 2: note = H or Y
  VARIANT 4: note = G, T, V, K or N
  VARIANT 6: note = R or K
TXGXAXSVRL

SEQ ID NO: 88 - protein, 17 aa, synthetic construct
  VARIANT 5: note = N or R
  VARIANT 7: note = E, N, I or Y
  VARIANT 8: note = N or Y
  VARIANT 9: note = A or S
  VARIANT 12: note = S, R or A
  VARIANT 13: note = D or E
  VARIANT 14: note = S, D, F or T
  VARIANT 16: note = M or H
LLDWXGXXXEYXXXVXG

SEQ ID NO: 89 - protein, 10 aa, synthetic construct
  VARIANT 2: note = H or Y
  VARIANT 4: note = G, T, V, K or N
  VARIANT 6: note = R or K
  VARIANT 10: note = S, L, K or A
TXGXAXSVRX

SEQ ID NO: 90 - protein, 17 aa, synthetic construct
  VARIANT 3: note = S or D
  VARIANT 7: note = E, N, I or Y
  VARIANT 8: note = N or Y
  VARIANT 9: note = A or S
  VARIANT 12: note = S, R or A
  VARIANT 13: note = D or E
  VARIANT 14: note = S, D, F or T
  VARIANT 16: note = M or H
LLXWRGXXXEYXXXVXG

SEQ ID NO: 91 - protein, 17 aa, synthetic construct
  VARIANT 3: note = S or D
  VARIANT 5: note = N or R
  VARIANT 7: note = N, I or Y
  VARIANT 8: note = N or Y
  VARIANT 9: note = A or S
  VARIANT 12: note = S, R or A
  VARIANT 13: note = D or E
  VARIANT 14: note = S, D, F or T
  VARIANT 16: note = M or H
LLXWXGXXXEYXXXVXG

SEQ ID NO: 92 - protein, 17 aa, synthetic construct
  VARIANT 3: note = S or D
  VARIANT 5: note = N or R
  VARIANT 7: note = E, N, I or Y
  VARIANT 9: note = A or S
  VARIANT 12: note = S, R or A
  VARIANT 13: note = D or E
  VARIANT 14: note = S, D, F or T
  VARIANT 16: note = M or H
LLXWXGXYXEYXXXVXG

SEQ ID NO: 93 - protein, 17 aa, synthetic construct
  VARIANT 3: note = S or D
  VARIANT 5: note = N or R
  VARIANT 7: note = E, N, I or Y
  VARIANT 8: note = N or Y
  VARIANT 12: note = S, R or A
  VARIANT 13: note = D or E
  VARIANT 14: note = S, D, F or T
  VARIANT 16: note = M or H
LLXWXGXXSEYXXXVXG

SEQ ID NO: 94 - protein, 17 aa, synthetic construct
  VARIANT 3: note = S or D
  VARIANT 5: note = N or R
  VARIANT 7: note = E, N, I or Y
  VARIANT 8: note = N or Y
  VARIANT 9: note = A or S
  VARIANT 12: note = R or A
  VARIANT 13: note = D or E
  VARIANT 14: note = S, D, F or T
  VARIANT 16: note = M or H
LLXWXGXXXEYXXXVXG

SEQ ID NO: 95 - protein, 17 aa, synthetic construct
  VARIANT 3: note = S or D
  VARIANT 5: note = N or R
  VARIANT 7: note = E, N, I or Y
  VARIANT 8: note = N or Y
  VARIANT 9: note = A or S
  VARIANT 12: note = S, R or A
  VARIANT 14: note = S, D, F or T
  VARIANT 16: note = M or H
LLXWXGXXXEYXEXVXG

SEQ ID NO: 96 - protein, 10 aa, synthetic construct
  VARIANT 2: note = H or Y
  VARIANT 4: note = G, T, V, K or N
  VARIANT 6: note = R or K
  VARIANT 10: note = S, L, K or A
TXGXAXSVRX

SEQ ID NO: 97 - protein, 17 aa, synthetic construct
  VARIANT 3: note = S or D
  VARIANT 5: note = N or R
  VARIANT 7: note = E, N, I or Y
  VARIANT 8: note = N or Y
  VARIANT 9: note = A or S
  VARIANT 12: note = S, R or A
  VARIANT 13: note = D or E
  VARIANT 14: note = S, D, F or T
LLXWXGXXXEYXXXVHG

SEQ ID NO: 98 - protein, 10 aa, synthetic construct
  VARIANT 4: note = G, T, V, K or N
  VARIANT 6: note = R or K
  VARIANT 10: note = S, L, K or A
TYGXAXSVRX

SEQ ID NO: 99 - protein, 10 aa, synthetic construct
  VARIANT 2: note = H or Y
  VARIANT 4: note = G, T, V, K or N
  VARIANT 10: note = S, L, K or A
TXGXAKSVRX

SEQ ID NO: 100 - protein, 10 aa, synthetic construct
GGGGSGGGGS

SEQ ID NO: 101 - protein, 5 aa, synthetic construct
GGGGS

SEQ ID NO: 102 - protein, 371 aa, synthetic construct
QVQLQESGGGLVQPGGSTRLSCVSSRTFSYYDMGWFRQAPGKEREFVALLSWRGNNAEYSDSVMGRFTVSRGNTQNSVNLQMNNLKPEDTGIYYCAVTYGGARSVRSWGQGTQVTVSSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 103 - protein, 577 aa, synthetic construct
QVQLQESGGGLVQPGGSTRLSCVSSRTFSYYDMGWFRQAPGKEREFVALLSWRGNNAEYSDSVMGRFTVSRGNTQNSVNLQMNNLKPEDTGIYYCAVTYGGARSVRSWGQGTQVTVSSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 104 - protein, 213 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 577 aa, synthetic construct
QVQLQESGGGLVQPGGSTRLSCVSSRTFSYYDMGWFRQAPGKEREFVALLSWRGNNAEYSDSVMGRFTVSRGNTQNSVNLQMNNLKPEDTGIYYCAVTYGGARSVRSWGQGTQVTVSSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 213 aa, synthetic construct; identical to SEQ ID NO: 104

SEQ ID NO: 107 - protein, 355 aa, synthetic construct
QVQLQESGGGLVQPGGSTRLSCVSSRTFSYYDMGWFRQAPGKEREFVALLSWNGENAEYSDSVMGRFTVSRGNTQNSVNLQMNNLKPEDTGIYYCAVTHGGARSVRSWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 355 aa, synthetic construct
QVQLQESGGGLVQPGGSTRLSCVSSRTFSYYDMGWFRQAPGKEREFVALLSWRGNNAEYSDSVMGRFTVSRGNTQNSVNLQMNNLKPEDTGIYYCAVTYGGARSVRSWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 118 aa, synthetic construct
QVQLQESGGGLVQPGGSTRLSCVSSRTFSYYDMGWFRQAPGKEREFVALLSWRGNNAEYSDSVMGRFTVSRGNTQNSVNLQMNNLKPEDTGIYYCAVTYGGARSVRSWGQGTQVTVSS

SEQ ID NO: 110 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 111 - protein, 50 aa, synthetic construct
  VARIANT 1..50: note = This sequence may encompass 1-10 SGGGG repeating units
SGGGGSGGGGSGGGGSGGGGSGGGGSGGGGSGGGGSGGGGSGGGGSGGGG

SEQ ID NO: 112 - protein, 54 aa, synthetic construct
  VARIANT 5..54: note = This region may encompass 1-10 SGGGG repeating units
GGGGSGGGGSGGGGSGGGGSGGGGSGGGGSGGGGSGGGGSGGGGSGGGGSGGGG

SEQ ID NO: 113 - protein, 32 aa, synthetic construct
  VARIANT 3..32: note = This region may encompass 1-10 SGG repeating units
GGSGGSGGSGGSGGSGGSGGSGGSGGSGGSGG

SEQ ID NO: 114 - protein, 6 aa, synthetic construct
HHHHHH